(19) United States
(12) Patent Application Publication
(10) Pub. No.: US 2019/0264197 A1
(43) Pub. Date: Aug. 29, 2019

(54) DISULFIDE-RICH PEPTIDE LIBRARIES AND METHODS OF USE THEREOF

(71) Applicant: Protagonist Therapeutics, Inc., Newark, CA (US)

(72) Inventors: David BARKAN, Richmond, CA (US); Tran Trung TRAN, Newark, CA (US); Mark Leslie SMYTHE, Bardon, Queensland (AU)

(21) Appl. No.: 16/319,959

(22) PCT Filed: Jul. 27, 2017
(86) PCT No.: PCT/US2017/044222

§ 371 Date: Jan. 23, 2019

Related U.S. Application Data
(60) Provisional application No. 62/367,550, filed on Jul. 27, 2016.

(51) Int. Cl. C12N 15/10; C40B 40/02; C40B 40/10; G16B 30/00; G16B 40/00; G16B 20/00; G16B 35/20; G16B 15/30

(52) U.S. Cl. CPC C12N 15/1044; C40B 40/02; C40B 40/10; G16B 30/00; C12N 15/1065; G16B 20/00; G16B 35/20; G16B 15/30; G16B 40/00

(57) ABSTRACT

Provided herein are libraries of structurally diverse disulfide-rich peptides (DRPs) and related methods of screening these libraries to identify DRPs that bind to a desired target.

### CROSS REFERENCE TO RELATED APPLICATIONS

This application claims priority to U.S. Provisional Application No. 62/367,550, filed on Jul. 27, 2016; which is hereby incorporated by reference herein in its entirety.

### SEQUENCE LISTING

The Sequence Listing associated with this application is provided in text format in lieu of a paper copy, and is hereby incorporated by reference into the specification. The name of the text file containing the Sequence Listing is PRTH_019_01WO_ST25.txt. The text file is 277 KB, was created on Jul. 13, 2017, and is being submitted electronically via EFS-Web.

### FIELD OF THE INVENTION

The present invention relates generally to libraries of structurally diverse disulfide-rich peptides (DRPs) and related methods of screening these libraries to identify DRPs that bind to a desired target.

### BACKGROUND OF THE INVENTION

In the past decade of drug discovery, peptide-based drugs have gathered momentum as a class of therapeutics, with their global market impact expected to increase significantly in the future [1]. Previously, the spectrum of available drugs consisted primarily of small molecules that target deep binding pockets on proteins to inhibit enzyme function. However, small molecules are generally not well-suited for binding to large, flat surfaces on a protein to inhibit protein-protein binding, a process that is critical for treating many human diseases [2]. In addition, small molecules frequently lack binding specificity, a disadvantage that can lead to failure in the development pipeline or to adverse side effects, even among drugs on the market [3]. In contrast, biologic-based drugs, such as monoclonal antibodies, have been found to be highly specific and effective blockers of protein-protein interactions, and their clinical use has transformed medicine over the past decade. Despite the growing success of antibody-based drugs, they do have several limitations. They are large and complex macromolecules that need to be delivered by injection, have long circulating half-lives with little ability to control drug levels in patients precisely, leading to safety consequences, and lack durability with patients losing response due to immunogenicity.

Peptides, in contrast to proteins, are generally regarded as being composed of up to 50 amino acids and lack a hydrophobic core [5]. The simplest peptides are linear and disordered, assuming structure only upon binding to a protein, and are prone to degradation by host factors. Thus, peptide drug design strategies often seek to engineer structure into the molecule [6]. These approaches include induction of secondary structure such as β-turns, α-helices and β-hairpins into the peptide [7]; head-to-tail cyclization [8, 9]; and incorporating non-natural amino acids as in peptoids [10]. Of particular interest is the use of disulfide bonds cross-linking cysteine residues that are distantly separated along the sequence to create a peptide fold to generate disulfide-rich peptides, or DRPs, which typically consist of up to 50 residues with between one and four disulfide bonds.

Disulfide-rich peptides (DRPs) are found throughout nature and are ideal scaffolds for drug development, because they are small peptides possessing a disulfide-strained core that imparts extraordinary chemical and biological stability. However, a challenge in developing a DRP therapeutic is to engineer the desired activity into the DRP scaffold to bind a specific target. The large sequence space sampled in a phage display library can help overcome this challenge. However, a lack of structural complementarity between the scaffold and the protein target may result in no peptide binders, regardless of the sequences displayed. Clearly, there is a need in the art for novel DRP libraries to increase the probability of finding a hit against any specific target.

### SUMMARY OF THE INVENTION

In one embodiment, the present invention includes a system or kit, e.g., a master library, comprising two or more disulfide-rich peptide (DRP) scaffold libraries, wherein each of the two or more DRP scaffold libraries comprises: (a) a plurality of DRPs comprising at least two cysteine residues capable of forming an intramolecular disulfide bond; or (b) a plurality of polynucleotides encoding the plurality of DRPs, wherein the plurality of DRPs of each DRP scaffold library share one or more common three-dimensional polypeptide structural feature. In certain embodiments, the one or more common three-dimensional polypeptide structural feature is different for each of the DRP scaffold libraries. In certain embodiments, the system or kit comprises three or more, five or more, ten or more, or twenty or more DRP scaffold libraries. In particular embodiments, each of the DRP scaffold libraries comprises at least 10⁵, at least 10⁶, at least 10⁷, at least 10⁸, at least 10⁹ or at least 10¹⁰ polypeptides. In certain embodiments, at least one of the one or more common three-dimensional polypeptide structural feature is a polypeptide surface feature or a core feature. In certain embodiments, at least one of the one or more common three-dimensional polypeptide structural feature is based on structural similarity and/or disulfide bond conservation. In certain embodiments, disulfide bond conservation is based on a distance between disulfide bonds of about 1.5 Å to about 2.5 Å. In certain embodiments, the distance between disulfide bonds is about 2.0 Å. In certain embodiments, the common three-dimensional polypeptide structural feature of each DRP scaffold library is depicted in FIG. 4. In certain embodiments, each of the one or more common three-dimensional polypeptide structural features is characterized as or is shared by one of the following polypeptide groups: knottin 1, knottin 2, insulin, small conotoxin, knottin 3, small hairpin, EGF-like hairpins, medium conotoxin, α-defensin, β-defensin, large hairpin, crambin, helix-loop-helix, LDL receptor, knottin IV, PMP inhibitors, TNF receptor, large conotoxin, tryptase inhibitor, and anti-microbial peptide. In certain embodiments, the plurality of DRPs of each DRP scaffold library are variants of a representative DRP. In certain embodiments, the plurality of DRPs within each DRP scaffold library have at least 30% identity to a representative DRP amino acid sequence for each DRP scaffold library. In certain embodiments, the plurality of DRPs within each DRP scaffold library have an average native overlap of at least 0.5 with a representative DRP amino acid sequence for each DRP scaffold library. In certain embodiments, the representative DRP amino acid sequence for each DRP scaffold library is an amino acid sequence shown in FIG. 8. In certain embodiments, the representative DRP amino acid sequence for each DRP scaffold library is an amino acid sequence shown FIG. 9, wherein X indicates any amino acid. In certain embodiments, the plurality of DRPs within each DRP scaffold library comprise a sequence having at least 80% identity to a sequence shown in FIG. 8 or FIG. 9, wherein X indicates any amino acid. In certain embodiments, the plurality of DRPs within each of the DRP scaffold libraries have an average native overlap of less than 0.5 with the consensus DRP amino acid sequence of other DRP scaffold libraries. In certain embodiments, the plurality of the DRPs within each of the DRP scaffold libraries comprise one or more amino acid modifications as compared to the representative DRPs, or wherein a plurality of polynucleotides within each of the DRP scaffold libraries encode DRPs comprising one or more amino acid modifications as compared to the representative DRPs. In certain embodiments, the one or more amino acid modifications comprise one or more amino acid additions, deletions or substitutions. In certain embodiments, the libraries are surface display libraries, and wherein the plurality of DRPs of each DRP scaffold library are fused to a cell surface polypeptide. In certain embodiments, the cell surface polypeptide is a cell surface polypeptide of a microorganism. In certain embodiments, the libraries are phage display libraries, and the plurality of DRPs are fused to a polypeptide displayed on a phage cell surface. In certain embodiments, the libraries are yeast display libraries, and the plurality of DRPs are fused to a polypeptide displayed on a phage cell surface. In certain embodiments, a plurality of the DRPs are capable of binding to a target polypeptide when expressed on the cell surface. In certain embodiments, the polynucleotides encode fusion polypeptides comprising each of the DRPs present in each of the DRP scaffold libraries fused to a cell surface polypeptide. In certain embodiments, the polynucleotides are expression vectors.

In a related embodiment, the present invention includes a method of identifying a disulfide-rich peptide (DRP) that specifically binds to a target polypeptide, comprising: (a) contacting the target polypeptide with the system or two or more disulfide-rich peptide (DRP) scaffold libraries of the present invention; and (b) detecting an amount of binding of the target polypeptide to a first DRP of a DRP scaffold library, wherein if the amount of binding of the first DRP to the target polypeptide is greater than the amount of binding of the first DRP to a control polypeptide, the first DRP specifically bind to the target polypeptide. In certain embodiments, the target polypeptide and/or the first DRP is labelled with a detectable label.

In another related embodiments, the present invention includes method of generating two or more disulfide-rich peptide (DRP) scaffold libraries, wherein each of the two or more DRP scaffold libraries comprises: (i) a plurality of DRPs comprising at least two cysteine residues capable of forming an intramolecular disulfide bond; or (ii) a plurality of polynucleotides encoding the plurality of DRPs, wherein the plurality of DRPs of each DRP scaffold library share a common three-dimensional polypeptide structural feature, the method comprising: (a) identifying two or more groups of DRPs comprising disulfide bonds, wherein the DRPs of each group share a different three-dimensional polypeptide structural feature; (b) identifying a consensus DRP within each of the two or more groups of DRP, optionally wherein the peptides within each of the groups have an average native overlap of at least 0.5 with the consensus peptide of the group and/or an average native overlap of less than 0.5 with the consensus peptides of other groups; (c) for each group of DRPs, producing a plurality of DRPs having at least 30% sequence identity to the consensus DRP of the group and comprising one or more amino acid modifications as compared to the consensus DRP, wherein each of the plurality of DRPs constitutes a disulfide-rich DRP scaffold library. In certain embodiments, the plurality of peptides of (c) are fused in-frame to a cell surface polypeptide.

In a further related embodiments, the present invention includes a method for identifying two or more clusters of disulfide-rich peptides (DRPs), comprising: (a) identifying in a protein database a plurality of DRPs comprising less than 50 amino acid residues and comprising at least one disulfide bond; (b) optionally removing duplicate DRPs from the plurality of DRPs identified in (a); (c) clustering the plurality of DRPs into two or more clusters based on peptide structural homology; (d) optionally reclustering knottin DRPs based on core disulfide bond structure; and (e) optionally re-assigning DRPs in less-populated clusters to other clusters, thus identifying two or more clusters of DRPs, wherein the DRPs of each cluster share a common three-dimensional polypeptide structural feature. In certain embodiments, the clustering of step (c) is performed using a clustering algorithm. In certain embodiments, the clustering algorithm is an average-linkage hierarchical clustering algorithm wherein the DRPs are clustered using native overlap as a distance metric, and wherein the algorithm is terminated when the smallest average native overlap between any two clusters is below a cutoff. In certain embodiments, the cutoff is 0.7. In certain embodiments, the reclustering of step (d) is performed using a clustering algorithm. In certain embodiments, the clustering algorithm is an average-linkage hierarchical clustering algorithm wherein the knottin DRPs are clustered using the distance between equivalent disulfide bonds as a distance metric, and wherein the algorithm is terminated when the distance between any two clusters is below a cutoff. In certain embodiments, the cutoff is 2.0 Å. In certain embodiments, the less-populated clusters consist of less than 10, less than 5, or 1 DRP.

### BRIEF DESCRIPTION OF THE DRAWINGS

FIGS. 1A and 1B provide diagrams depicting certain aspects of the present invention. FIG. 1A is a diagram of pipeline workflow. FIG. 1B shows an example of hierarchical clustering, portrayed as a tree where the leaves are DRPs and each inner node represents a cluster containing all DRPs in the sub-tree rooted at that node. Numbers at the branch point are the values of the distance metric when calculated across the two sub-trees which are being merged at the inner node. The dashed line is the empirically selected cutoff; all sub-trees to the right of this cutoff represent the final clusters.

FIGS. 2A-2F depict visualization of clusters identified during the determination of clustering cutoffs. The top row shows the resulting clusters following the initial native overlap hierarchical clustering step. Each image represents a different cutoff applied for determining the final clusters for that step. These images informed the decision of which cutoff to impose in the final protocol. (FIG. 2A) Conotoxin and small hairpin clusters at the native overlap cutoff of 0.7, which was ultimately selected as the final cutoff. (FIG. 2B) At a cutoff of 0.6, the same conotoxin and small hairpin DRPs were assigned to the same cluster despite assuming different secondary structures. (FIG. 2C) At a cutoff of 0.8, conotoxin DRPs were assigned to separate clusters despite each cluster fold consisting of circular loops and short helical regions. The bottom row shows the resulting clusters following the knottin reclustering step, with each image representing the knottin cluster containing the most DRPs after applying a different cutoff. Only the disulfide bonds in the DRPs are displayed, in light gray. The cutoffs assessed were (FIG. 2D) 2.0 Å RMSD, (FIG. 2E) 1.5 Å RMSD, and (FIG. 2F) 2.5 Å RMSD. 2.0 Å was selected as the optimal cutoff and used in the final protocol.

FIG. 3 is a graph showing cluster DRP coverage. Clusters were sorted by size from most to least populated and each cluster was assigned an index starting with 1. At each index i, the cumulative number of DRPs in that cluster and all clusters with index less than i was calculated and divided by the total number of DRPs in the dataset, resulting in the coverage. Coverage as a function of index is displayed. Coverage curves are shown after completion of successive steps of the procedure (lines from top to bottom: shorter singletons, longer singletons, merged knottins, native overlap).

FIG. 4 displays the top 20 clusters by size. Singleton DRPs are removed for clarity (images of clusters including singletons are available in FIG. 12). DRPs are colored according to sequence conservation within the cluster, ranging from light gray (high conservation) to dark gray (moderate) to medium gray (low conservation). Regions containing disulfide bonds are circled. The core structure associated with each cluster is referred to as: (1) knottin 1, (2) knottin 2, (3) insulin, (4) small conotoxin, (5) knottin 3, (6) small hairpin, (7) EGF-like hairpins, (8) medium conotoxin, (9) α-defensin, (10) β-defensin, (11) large hairpin, (12) crambin, (13) helix-loop-helix, (14) LDL receptor, (15) knottin IV, (16) PMP inhibitors, (17) TNF receptor, (18) large conotoxin, (19) tryptase inhibitor, or (20) anti-microbial peptide.

FIGS. 5A-5E provides the results of a phage display experiment. (FIG. 5A) Structure of the peptide scaffold for phage library IKATr1. Variable residue positions are colored in dark gray, and disulfide bonds in light gray. The same representation is used for the 1KVFr1 (FIG. 5B) and 1ZDCr1 (FIG. 5C) library scaffolds. (FIG. 5D) Enrichment ratios across successive rounds of phage panning for the three libraries, with bars for each panning round representing IKATr1, 1KVFr1 and 1ZDCr1 from left to right. Panning was discontinued after the fourth round for IKATr1 and 1KVFr1 due to a lack of enrichment. (FIG. 5E) Standard curve resulting from competition ELISA experiment to assess inhibition of IL-23/IL-23R complex formation by the identified clone (Peptide 1).

FIG. 6 is a table showing the initial structural classification of proteins (SCOP) folds and SCOP annotation for peptides in each cluster following the initial native overlap clustering step. For each cluster index, the table indicates the SCOP folds, the fold names, and the count.

FIG. 7 is a table depicting knottin clusters before and after reclustering reduction. Following the initial native overlap clustering step, all clusters containing four or more knottins were merged and their peptides reclustered according to the structural overlap of their disulfide bonds. Columns represent the indices of the thirteen knottin clusters prior to reclustering, and rows represent the four final knottin clusters after reclustering. Each cell shows the number of DRPs in the initial cluster that were assigned to the final cluster, as well as the total number of DRPs in the initial cluster (for example, 36 out of 37 knottins in the fourth initial cluster were assigned to the second final cluster).

FIG. 8 is a table providing the complete composition of each cluster; where each row represents one DRP. Cluster in DRP Name: name of the DRP. Cluster Name: Manually assigned name of the cluster, derived from the dominant SCOP fold observed among peptides in that cluster. Distance to Centroid: Native overlap between the DRP and the cluster centroid. Uniprot Accession: Uniprot accession of the DRP when a mapping could be made between the PDB entry and an entry in Uniprot. PDB Sequence: Amino acid residue sequence of the DRP in the PDB entry (SEQ ID NO:6 to SEQ ID NO:811). Disulfide Bond Count: Number of disulfide bonds in the DRP.

FIG. 9 is a table showing details on the design for the three phage libraries described in the Examples. Five rows are included for each library displaying the wild-type sequences (SEQ ID NOs: 812, 4, 815, 1, 818 and 2) of the selected DRP along with the diversified positions indicated with NNK (codon) (SEQ ID NOs: 813, 816 and 819) and X (Amino Acid) (SEQ ID NOs: 814, 817 and 3).

FIG. 10 is a table showing the number of distinct species found in each cluster, and the ratio of the number of DRPs to the number of species in each cluster.

FIG. 11 is a table showing the disulfide bond patterns observed in selected clusters, where CXnC represents n non-cysteine amino acid residues between two disulfide-bonded cysteines. Each row represents one pattern found in a cluster along with the number of DRPs in the cluster with that pattern.

FIG. 12 provides images of the 20 clusters, including the singletons. Peptides are represented as in FIG. 4.

### DETAILED DESCRIPTION OF THE INVENTION

### Abbreviations

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

Use of the term “comprising” is meant to also provides support for the narrower term “consisting of.”

The term “peptide,” as used herein, refers broadly to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring.

The term “amino acid” or “any amino acid” as used here refers to any and all amino acids, including naturally occurring amino acids (e.g., α-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The 20 “standard,” natural amino acids are listed in the above tables. The “non-standard,” natural amino acids are pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g. alanine, arginine, etc.), they are designated by their conventional three-letter or single-letter abbreviations (e.g. Ala or A for alanine, Arg or R for arginine, etc.). Unless otherwise indicated, three-letter and single-letter abbreviations of amino acids refer to the L-isomeric form of the amino acid in question. The term “L-amino acid,” as used herein, refers to the “L” isomeric form of a peptide, and conversely the term “D-amino acid” refers to the “D” isomeric form of a peptide (e.g., Dasp, (D)Asp or D-Asp; Dphe, (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations.

The recitations “sequence identity”, “percent identity”, “percent homology”, or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and nonhomologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM 120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

### DESCRIPTION

The present invention is based, in part, on the characterization of DRPs and the identification of groups of DRPs that share one or more common structural feature within each group. These groups may be referred to herein as DRP clusters. In addition, the present invention is also relates to the development of a plurality of distinct DRP scaffold libraries (also referred to as DRP cluster libraries), wherein each DRP scaffold library is based on a representative DRP within each of the identified groups of DRPs (i.e., DRP clusters). The representative DRP of a DRP cluster serves as a scaffold for producing a library of related DRPs that share one or more common structural features, which may include the presence of the disulfide bonds present in the representative DRP and/or any of the other structural features described herein. Each DRP scaffold library comprises a plurality of DRPs, wherein the plurality of DRPs comprise one or more amino acid modifications as compared to the representative DRP. While DRPs have been used as starting points for designing inhibitors of protein-protein interactions, modifying the DRP sequence to enable specific binding to a desired protein target remains a challenge. The present invention facilitates the screening of DRPs having a variety of different scaffolds, thus increasing the likelihood of identifying a DRP that binds to a target of interest.

The present invention further provides methods of identifying DRPs that bind to a target of interest, which include screening one, two or more DRP scaffold libraries of the present invention. A variety of methods may be used to screen DRP scaffold libraries of the present invention, some of which involve the expression of the DRP scaffold libraries on the surface of a microorganism, such as yeast display or phage display, which, in certain embodiments, can sample up to at least 10¹⁰ unique protein sequences and enables selection for those that bind the target.

In particular embodiments, the DRPs within a DRP scaffold library comprise one or more disulfide bonds. In particular embodiments, the DRPs within a DRP scaffold library comprise the same or a singular disulfide bond pattern, e.g., the same number of disulfide bonds, the same number of amino acid residues between the two amino acids that form a disulfide bond. Thus, in particular embodiments, a disulfide bond pattern present in the representative DRP (of a DRP cluster) that is used to generate a DRP scaffold library is conserved in the DRP members of the DRP scaffold library.

Many of the desirable properties of therapeutic compounds found in DRPs are demonstrated by their biological functions. They frequently assume the ‘knottin’ fold in which six or more cysteines form disulfide bonds in an interlocking arrangement, often incorporating head-to-tail cyclization [10]. These knottins have diverse functions ranging from plant defense [11] to incapacitating prey when expressed as toxins in venomous animals[12]. Knottins have been reported to show low-immunogenic potential [13], which avoids challenges often presented by other biologics, such as antibodies. Another fold class is small β-hairpins stabilized both by the standard backbone hydrogen-bond patterns as well as one or more disulfide bonds linking the paired β-strands. These hairpins are often natural protease inhibitors[14], or can be converted to such with simple modifications [15]. Other examples of DRPs in nature include anti-microbial defensins [16], small conotoxins [17], and insulin [18].

Disulfide bonds stabilize the fold of a peptide by decreasing the entropy of the system proportionally to the number of residues between the linked cysteines [19, 20]. This increased stability confers beneficial properties desirable in a drug, including enhanced potency, selectivity, permeability, thermal stability, resistance to denaturation at low pH, protection against proteolytic attack [21], and in some instances increased activity when delivered orally [22-25]. Disulfide bonds may lock the molecule into a conformation that is complementary to a protein target [26], providing an opportunity to engineer the surface with new functionality while maintaining the fold. For example, a number of studies have grafted the binding surface of a protein onto a DRP scaffold, resulting in a molecule that retains the advantages of DRPs while reproducing the binding properties of the original protein [27, 28]. Current drugs on the market incorporating disulfide bonds include insulin, orally delivered linaclotide for treating inflammatory bowel syndrome [29], ziconotide for treatment of pain [30], and pramlintide as an adjunct therapy for type II diabetes [31].

While DRPs are used as starting points for designing inhibitors of protein-protein interactions, modifying the DRP sequence to enable specific binding to a desired protein target remains a challenge. One potential solution is phage display, which can sample up to 10¹² unique protein sequences and allows for selection of those that bind the target [32]. In one form of this experiment, a DNA library encoding a peptide (e.g., a representative peptide of a DRP scaffold), with some or all of the codons randomized, is ligated into a phage plasmid in a gene encoding for a coat protein, resulting in a library of phage expressing diversified peptide sequences on their surface. The library is then introduced to an immobilized protein target in a procedure referred to as ‘panning’. Phage particles with peptides that bind the immobilized target are selected over those that do not and are subsequently washed away. The enriched population of clones expressing binding peptides is then amplified and the process is repeated in an iterative panning and amplification process. Finally, the selected phage clones, referred to as hits, are sequenced and the peptides corresponding to those sequences are synthesized and assayed to confirm binding. A number of studies have used DRPs as phage library scaffolds [33], and have reported the rationale design and development of potent IL-6 compounds using this method [34].

A drawback in phage display is that a single phage library may yield no hits when panned against a target, regardless of the sequences displayed in the library, due to (i) the possibility that none of the generated sequences is complementary to the target or (ii) the inability to select rare and weakly active phage clones in a large pool of inactives. Therefore, the present disclosure contemplates that the probability of obtaining a hit increases if multiple phage libraries encoding structurally distinct scaffolds are used. As more unique scaffolds are panned, it is increasingly likely that at least one of them will result in a sequence with sufficient affinity for binding the target. The challenge solved by the present invention is the selection of DRPs to use as phage library scaffolds. To reduce the odds of creating redundant phage libraries, the present disclosure provides structurally distinct scaffold DRPs that cover a large fraction of known DRP fold.

The present invention provides for grouping DRPs according to structural similarity and selecting a representative DRP from each DRP cluster, thus guaranteeing that the representative DRPs are structurally distinct. The representative DRPs should be small enough to make it experimentally tractable to construct a phage library using each representative DRP as a scaffold. In certain embodiments, the representative DRP has between 10 and 50 amino acids, or 11 to 49 amino acids. In certain embodiments, a fragment of a representative DRP, e.g., a fragment of 10 to 50 amino acid residues, or 11 to 49 amino acids, is used. Each DRP cluster should include as many DRPs as possible, thus allowing for a maximum estimation of the fraction of total DRP structural diversity covered by the representative DRP peptides. Finally, the method may be automated so that the clustering can be updated as more DRP structures are solved and added to the Protein Data Bank (PDB). However, the number of structural folds into which DRPs can be clustered is not known, so there is no guarantee that all of these properties can be achieved. There have been previous attempts to perform such clustering, but they were either focused on a subset of DRP fold classes or required significant manual intervention [35-37].

In one aspect, the present invention includes a DRP clustering protocol (e.g., an automated DRP clustering protocol) that incorporates structural similarity and disulfide-bond conservation to group related DRPs, accompanied by a metric to select a representative member from each DRP cluster to use as a scaffold for generating DRP libraries, e.g., yeast or phage display libraries. As described herein, the method was applied to the solved structures of DRPs deposited in the Protein Data Bank (PDB). By examining the resulting clusters, an understanding of the degree to which DRPs can be grouped together and how sequence conservation varies within each cluster was gained. DRPs structurally distinct from each other but similar to other DRPs in their clusters were identified and libraries of distinct DRP scaffolds were produced.

Previous approaches have been successful in engineering into a DRP the ability to bind a target, either through phage display [33], grafting the exact binding surface of a protein known to bind the target [27], or a combination of the two [38]. In certain embodiments, the present invention uses phage display to pan multiple DRP scaffolds possessing maximally structurally diverse binding surfaces to greatly increase the likelihood of finding an initial hit against a target. Separately, the present invention is also based on the hypothesis that, while DRP folds found in the PDB are likely not completely representative of all DRP folds found in nature, they do represent a large fraction, possibly even the majority of such folds, and thus the scaffolds of the present invention are representative of a similarly large fraction of possible DRP structural diversity. Therefore, especially considering their favorable chemical and biological stabilities, the phage libraries for these 20 representatives are a valuable resource for discovering DRPs interacting with protein targets.

The accompanying examples demonstrate experimentally the utility of the present invention. A hierarchical clustering protocol incorporating DRP structural similarity was developed and applied, followed by two post-processing steps, to classify 818 unique DRP structures into 81 clusters, with the 20 most populated clusters comprising 85% of all DRPs. Representative DRPs were selected from each of these clusters, which were structurally distinct from one another but similar to other DRPs in their respective clusters. A large number of different DRPs were generated by manipulating approximately 4-18 amino acids of each representative DRP in a topologically controlled, biologically relevant and defined structure space. Phage libraries were constructed from three of these representative DRPs (using each representative DRP as a scaffold for generating a DRP scaffold library) and panned against human Interleukin-23 (IL-23) cytokine protein, a clinically validated target involved in inflammatory bowel disease, which affects 0.5% of the world's population, psoriasis and other disorders. DRPs that bind to IL-23 were identified from one of the libraries, demonstrating that peptide libraries based on distinct DRP scaffolds have biologically relevant topologies, are structurally diverse between libraries, and are composed of a large number of sequences within each library, and as such are a valuable resource for hit and lead discovery. Further, when combined with a large variety of diverse chemistries at various scaffold position, the DRP scaffold libraries of the present invention provide a unique solution for the discovery of peptides that bind a target of interest, including agonists and antagonists of protein-protein interactions involved in human disease.

Disulfide-Rich Peptides

DRPs are peptides that comprise one or more disulfide bonds cross-linking cysteine residues that are distantly separated within the DRP sequence. In particular embodiments, two cysteine residues of a DRP that are cross-linked by a disulfide bond are separated by from 0 to 16 amino acid residues. DRPs typically consist of up to 50 residues (e.g., 10 to 50 amino acid residues) with between one and four disulfide bonds, which can cause the formation of a peptide fold within the DRP. Many of the desirable properties of therapeutic compounds found in DRPs are demonstrated by their broad applications in nature. They frequently assume the “cysteine-knot” fold, also known as knottins, in which six or more cysteines form disulfide bonds in an interlocking arrangement, often incorporating head-to-tail cyclization [11]. Knottins have a diverse set of functions ranging from plant defense [12] to incapacitating prey when expressed as toxins in venomous animals [13]. These peptides have been reported to show low-immunogenic potential [14], which avoids challenges presented in developing other biologics such as antibodies. Another fold class is small β-hairpins stabilized both by the standard backbone hydrogen-bond patterns as well as one or more disulfide bonds linking the paired β-strands. These hairpins are often natural protease inhibitors [15], or this property can be induced by simple modifications [16]. Other examples of DRPs in nature include anti-microbial defensins [17], small conotoxins [18], and insulin [19]. DRP fold classes include, but are not limited to: (1) knottin 1, (2) knottin 2, (3) insulin, (4) small conotoxin, (5) knottin 3, (6) small hairpin, (7) EGF-like hairpins, (8) medium conotoxin, (9) α-defensin, (10) β-defensin, (11) large hairpin, (12) crambin, (13) helix-loop-helix, (14) LDL receptor, (15) knottin IV, (16) PMP inhibitors, (17) TNF receptor, (18) large conotoxin, (19) tryptase inhibitor, and (20) anti-microbial peptide.

Disulfide bonds stabilize the fold of a peptide by decreasing the entropy of the system by a factor proportional to the distance along the sequence between the linked cysteines [20, 21]. This increased stability may result in enhanced potency, selectivity, permeability, and confer beneficial properties necessary in a drug, such as resistance to denaturation in low pH, enhanced thermal stability, protection against proteolytic attack [22], and in some instances activity when delivered orally. The peptide is constrained despite often lacking a hydrophobic core; in this fashion, disulfide bonds maintain or lock the molecule in a conformation that can bind to a protein target [23]. This provides an opportunity to engineer the surface with new functionality whilst maintaining the fold. Current drugs on the market incorporating disulfide bonds include insulin, Ironwood Pharmaceutical's orally delivered Linaclotide for treating inflammatory bowel syndrome [24], Jazz Pharmaceutical's Ziconotide for treatment of pain [25], and Amylin's Pramlintide as an adjunct therapy for type II diabetes [26].

DRP Clusters

The present invention also provides method for identifying clusters of disulfide-rich peptides (DRPs). In general, the method comprises identifying peptides having one or more disulfide bonds (optionally less than about 50 amino acids or 60 amino acids in length), determining the structure of the identified peptides, and identifying peptides having one or more shared structural features, thus identifying peptides within a cluster. In particular embodiments, two or more different groups of peptides, wherein each peptide within a group shares one or more structural features, are identified, where each group is a separate cluster having at least one or more distinct structural features, or combinations thereof, different from those of peptides in other clusters.

In certain embodiments, the method comprises identifying in a protein database a plurality of DRPs comprising at least one disulfide bond, wherein the DRPs are optionally less than about 50 amino acids or 60 amino acids in length. In particular embodiments, duplicate DRPs are removed before proceeding to determine peptide structures. In certain embodiments, the method further comprises determining an actual, predicted or putative structure for at least some of the DRPs, which may be determined using methods known in the art or described herein, e.g., NMR, X-ray crystallography, homology modeling, threading or molecular dynamics. In certain embodiments, the actual, experimental or predicted structure of the DRPs is already known. Each DRP is then assigned to a cluster based on peptide structural homology to other DRPs within the cluster. In certain embodiments, knottin DRPs are re-clustered based on core disulfide bond structure. In certain embodiments, singleton or other DRPs in less-populated clusters are reassigned to other clusters.

In particular embodiments, clustering and/or re-clustering is performed using a clustering algorithm. In certain embodiments, the clustering algorithm is an average-linkage hierarchical clustering algorithm wherein the DRPs are clustered using native overlap as a distance. In particular embodiments, the algorithm is terminated when the smallest average native overlap between any two clusters is below a cutoff. In particular embodiments, the cutoff is 0.6, 0.7, 0/8 or 0.9.

In certain embodiments, the re-clustering algorithm used to recluster knottin DRPs is an average-linkage hierarchical clustering algorithm wherein the knottin DRPs are clustered using the distance between equivalent disulfide bonds as a distance metric, and wherein the algorithm is terminated when the distance between any two clusters is below a cutoff. In particular embodiments, the cutoff is 1.0 Å, 2.0 Å or 3.0 Å. In particular embodiments, less-populated clusters consist of less than 10, less than 5, or 1 DRP.

In particular embodiments, all or substantially all of the DRPs within a DRP cluster (which may also be referred to herein as a DRP scaffold) have one or more shared structural features, such as a conserved helix, loop, sheet or dominant secondary structure. Loops are defined as any continuous amino acid sequence that joins secondary structural elements (e.g., helices and sheets). Consequently, loops are a superset of D-turns. Loops often play an important function as exemplified by their roles in ligand binding, DNA-binding, binding to protein toxin, forming enzyme active sites, binding of metal ions, binding of antigens by immunoglobulins, binding of mononucleotides and binding of protein substrates by serine proteases. An alpha-helix is the most common secondary structure of proteins [55] and play pivotal roles in many protein-protein interfaces [56].

In particular embodiments, DRP structure comparisons involves comparing intramolecular inter-residue distances [57], matching main-chain fragments [58], or Secondary Structure Elements (SSEs) [59], or other representations of the main chain, fold, secondary or tertiary structure know to the art. In certain embodiments, DRP cluster comparisons are performed using the SALIGN algorithm [49].

In particular embodiments, the shared structural feature is a DRP surface shape, which may be any three-dimensional property or feature of a DRP surface, such as may be described according to amino acid side chain location and orientation or by surface feature descriptor [60]. In particular embodiments, the DRP surface shape is of, comprises or derived from, a structural feature of a DRP. Such a structural feature may, for example, be a contact surface that interacts with another protein or other molecule such as a nucleic acid, nucleotide or nucleoside (e.g. ATP or GTP) carbohydrate, glycoprotein, lipid, glycolipid or small organic molecule (e.g. a drug or toxin) without limitation thereto. Therefore, for the purposes of exemplification, a domain may be a binding domain, such as, e.g., a ligand-binding domain of a receptor, a receptor binding domain of a ligand, a DNA-binding domain of a transcription factor, an ATP-binding domain of a protein kinase, chaperonin or other protein folding and/or translocation enzyme, a receptor dimerization domain or other protein interaction domains such as SH2, SH3 and PDB domains, or domains that bind small organic molecules or other molecules, although the skilled person will appreciate that the present invention is not limited to these particular examples. Structural features of DRPs may include loops, β-turns or other contact surfaces, helical regions, extended regions and other protein domains.

As used herein, “contact surfaces” are DRP surfaces having amino acid residues that contact or interact with another molecule, such as another protein. An example of a contact surface is the ligand-binding surface of a cytokine receptor, although without limitation thereto. Contact surfaces may be composed of one or more discontinuous and/or continuous surfaces. By “discontinuous protein surface” is meant a protein surface wherein amino acid residues are non-contiguous or exist in discontinuous groups of contiguous amino acid residues. In this regard, it will be appreciated that 3-turns and loops are examples of a “continuous protein surface”. That is, a protein surface that comprises a contiguous sequence of amino acids.

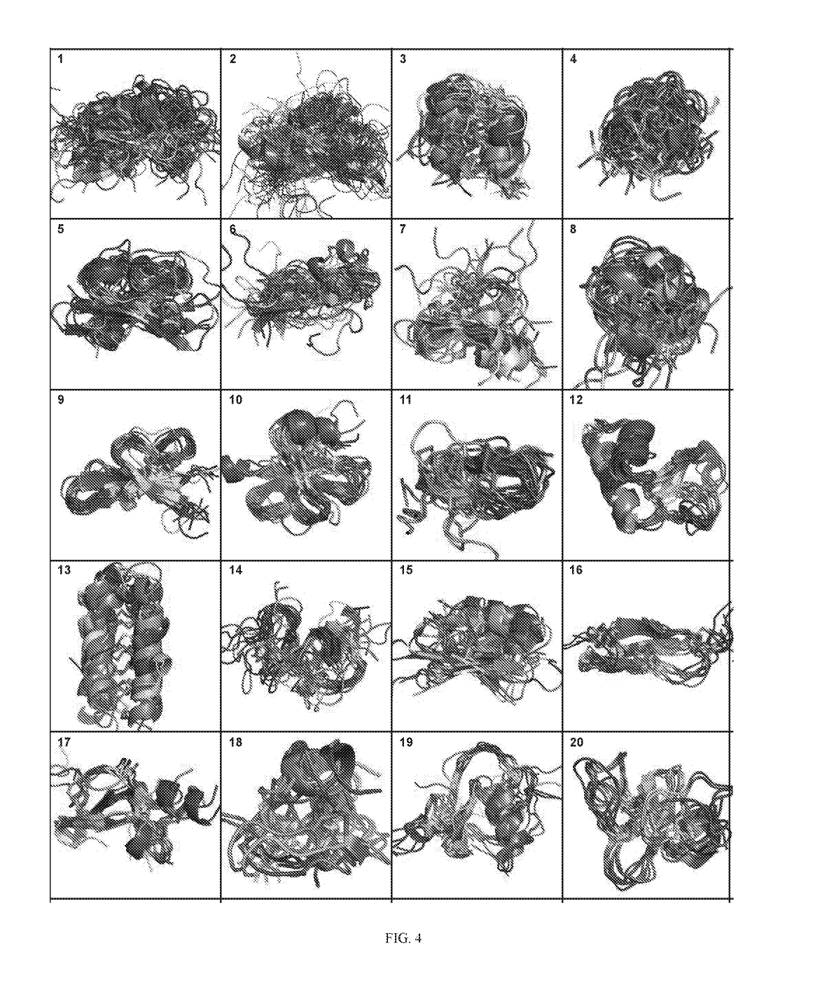

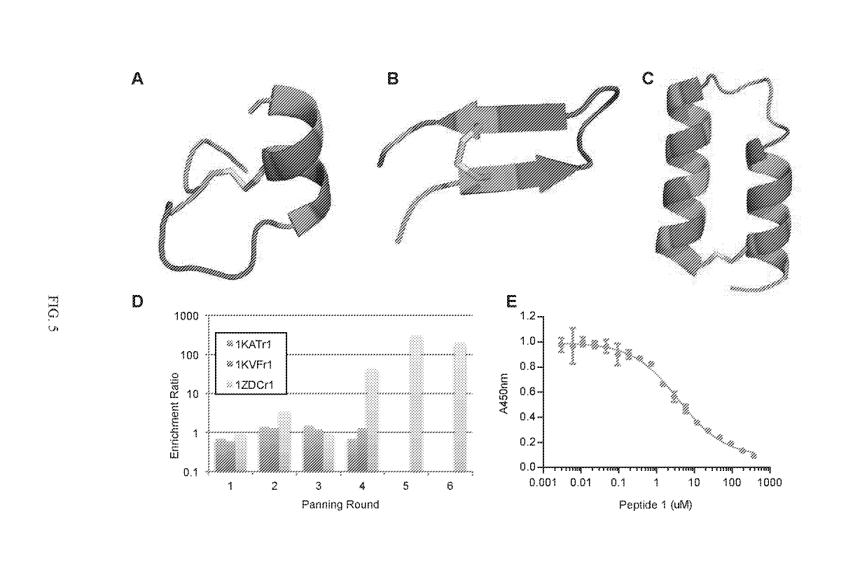

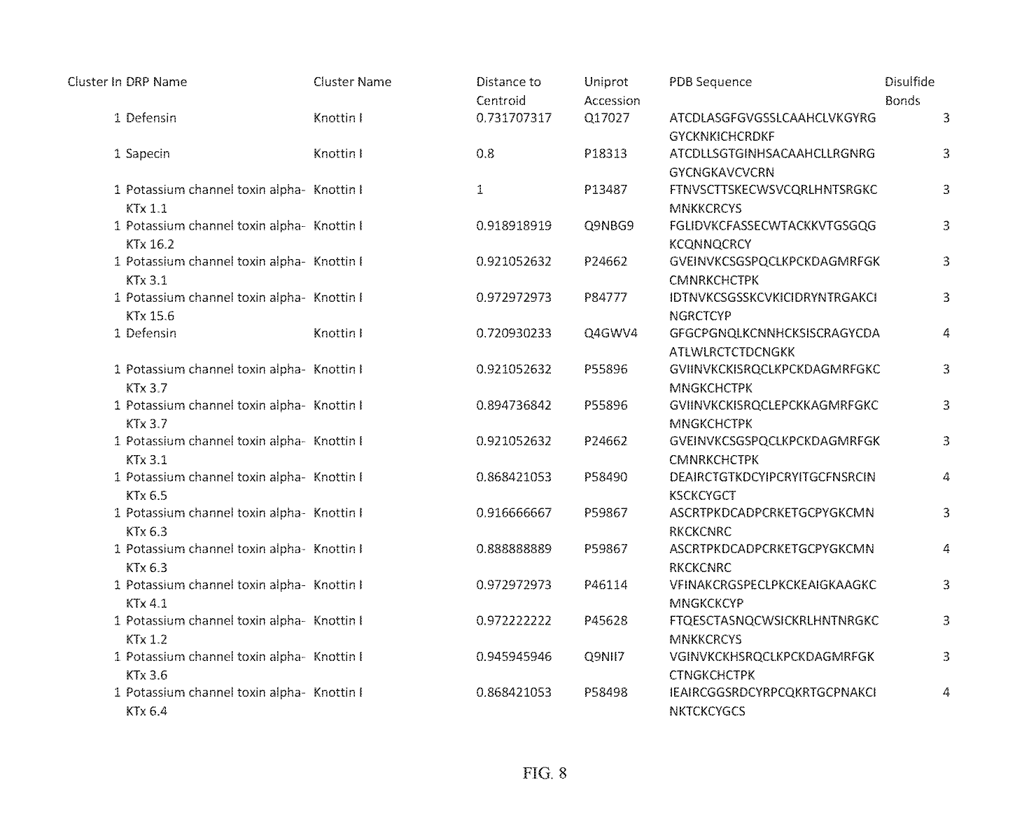

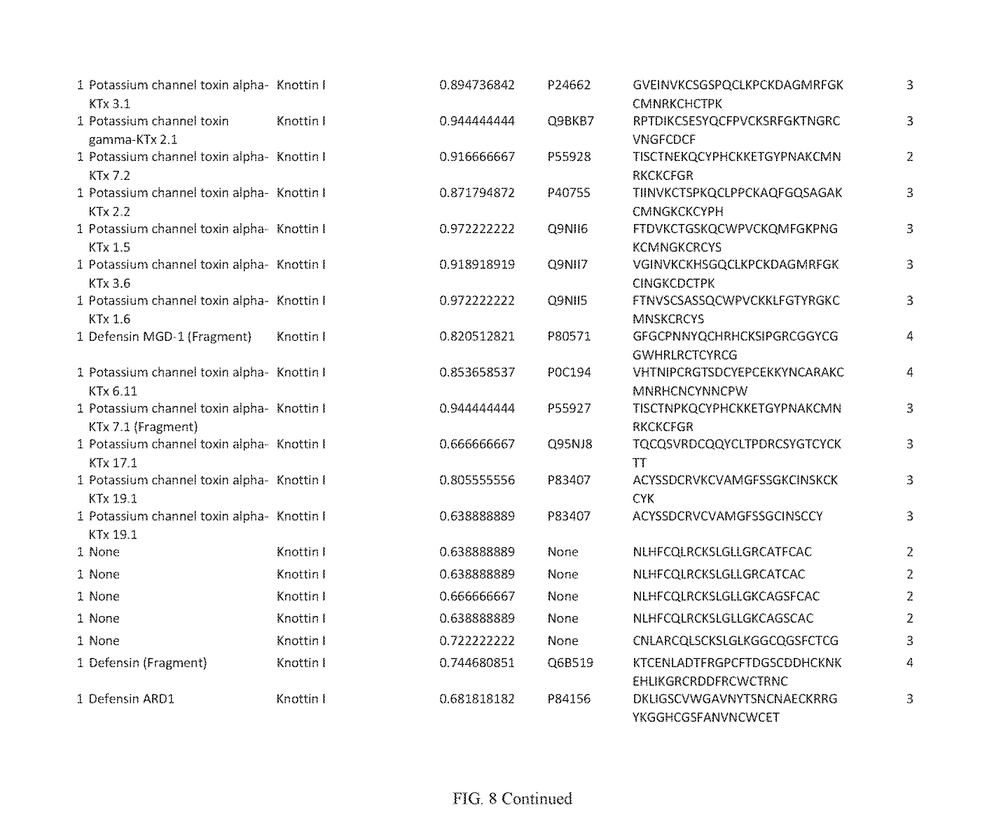

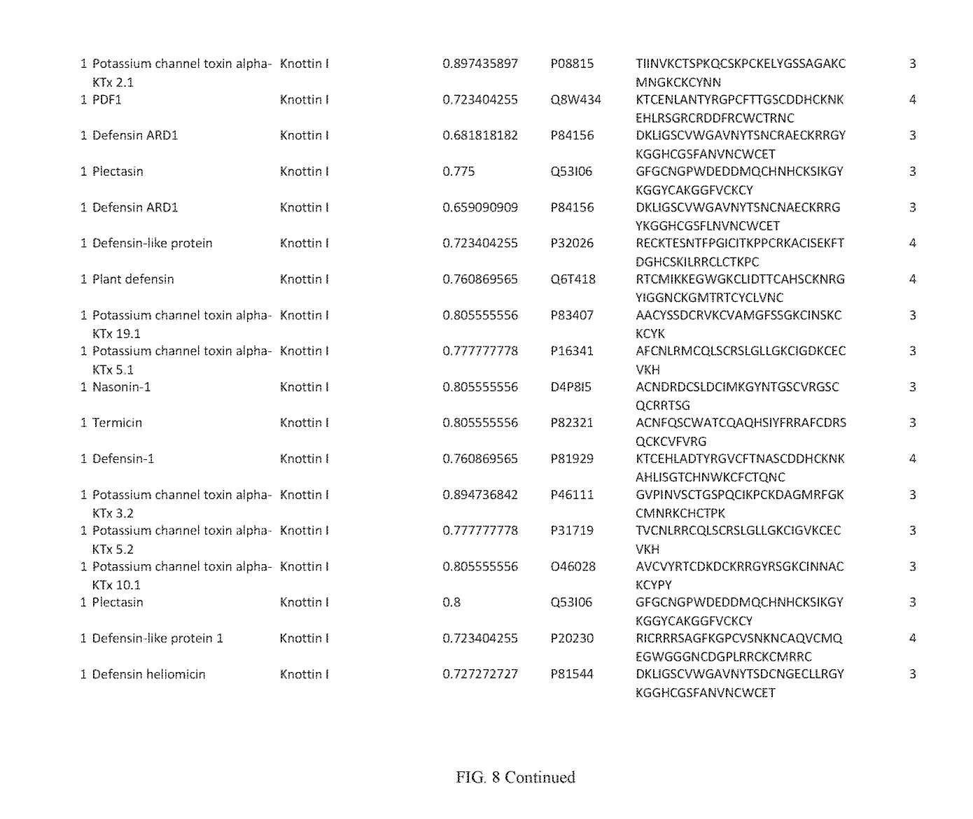

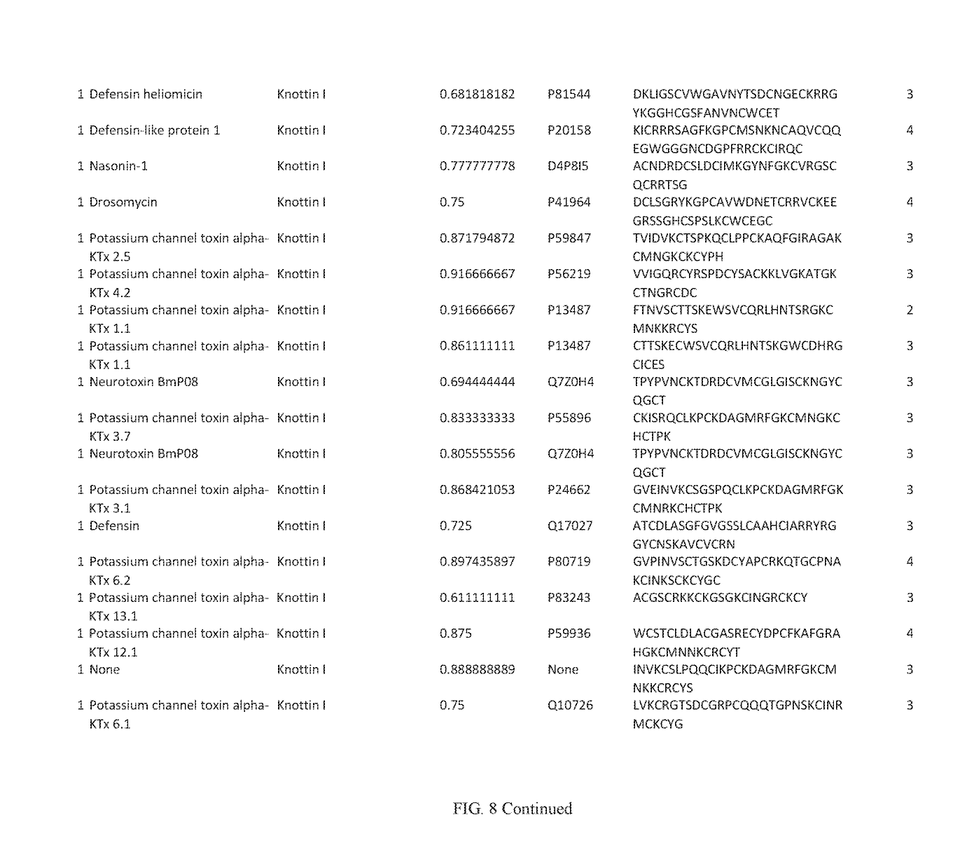

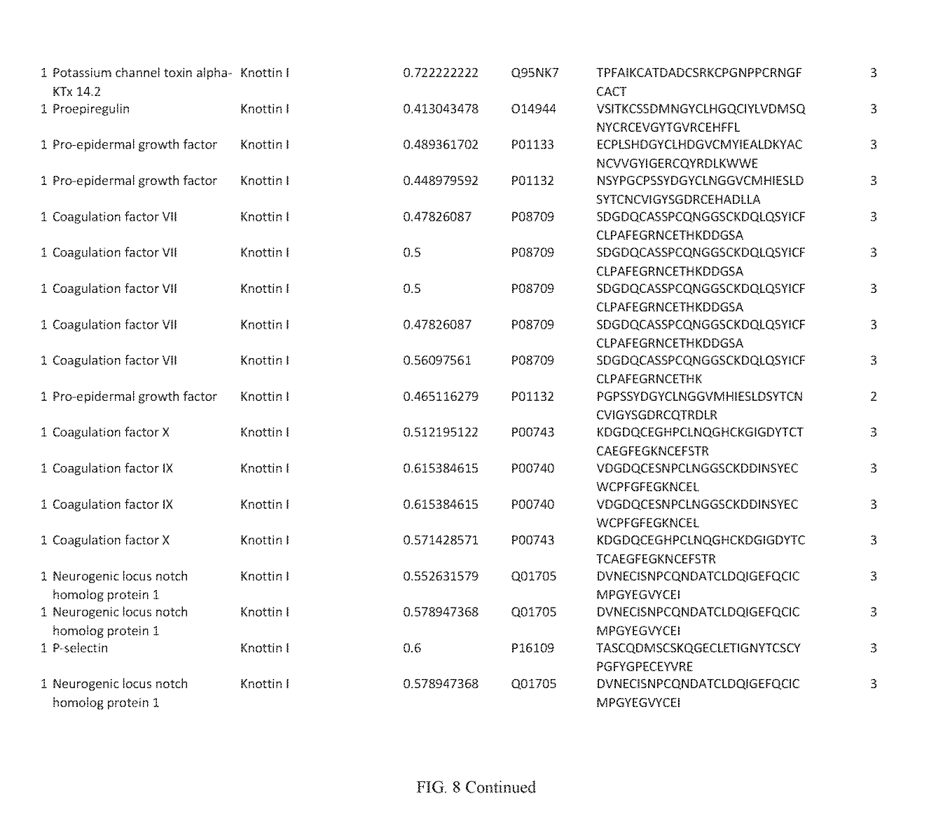

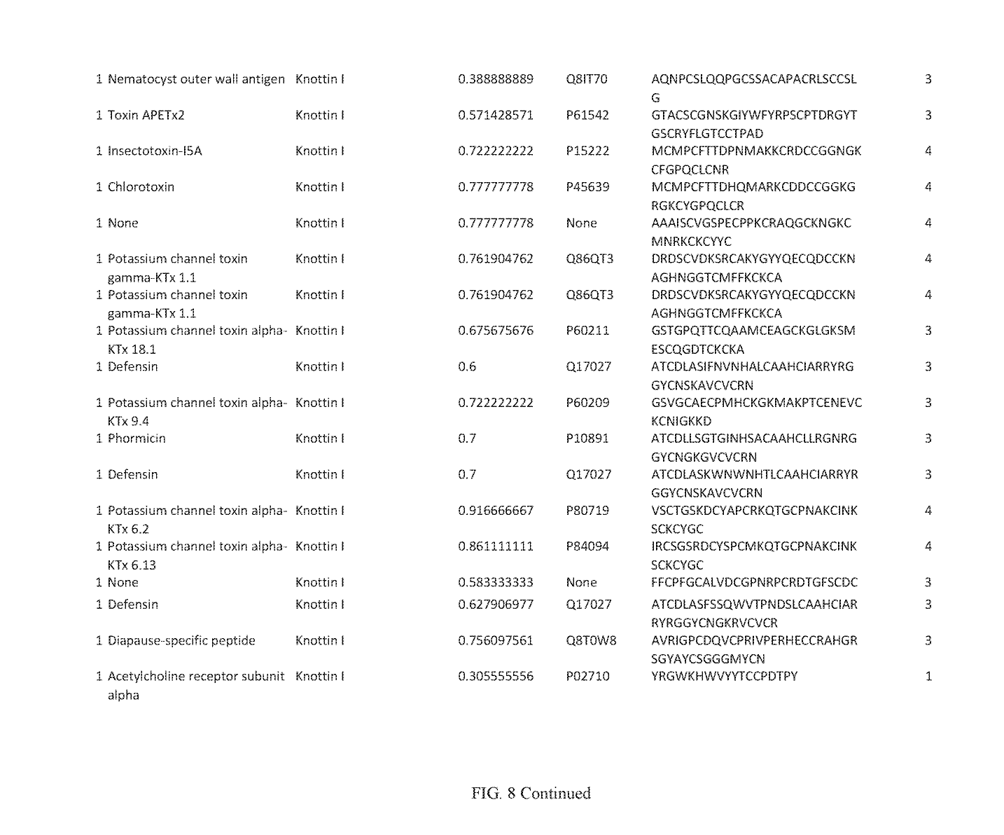

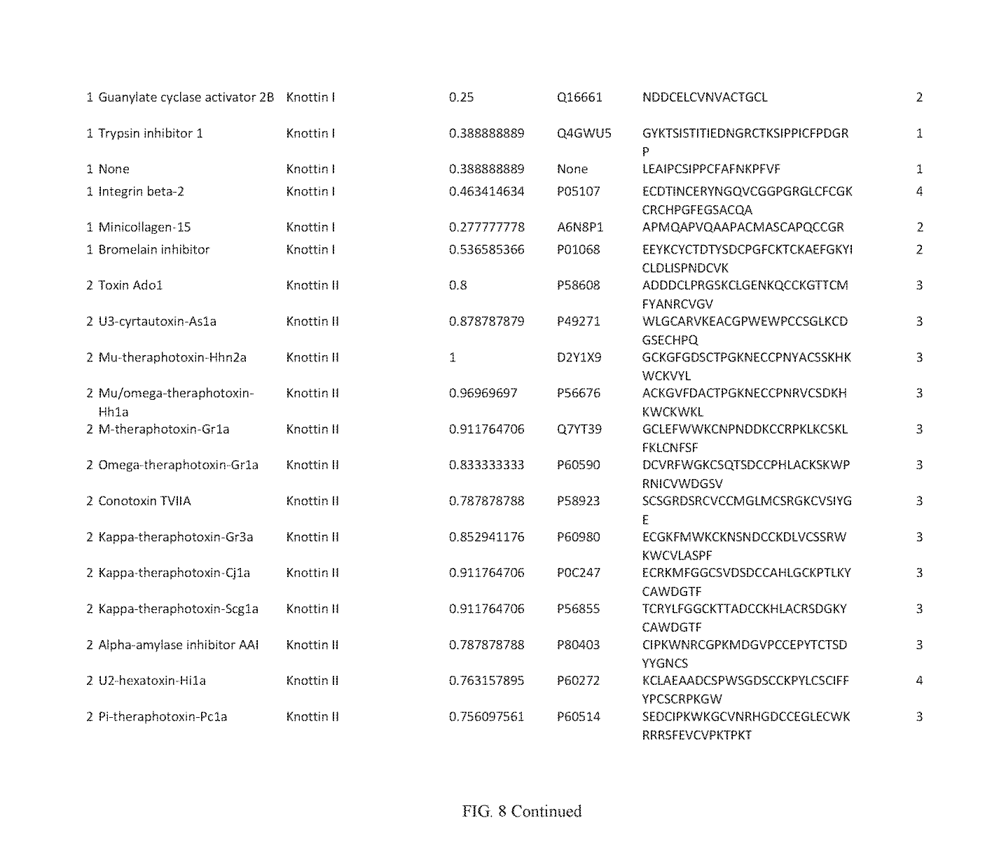

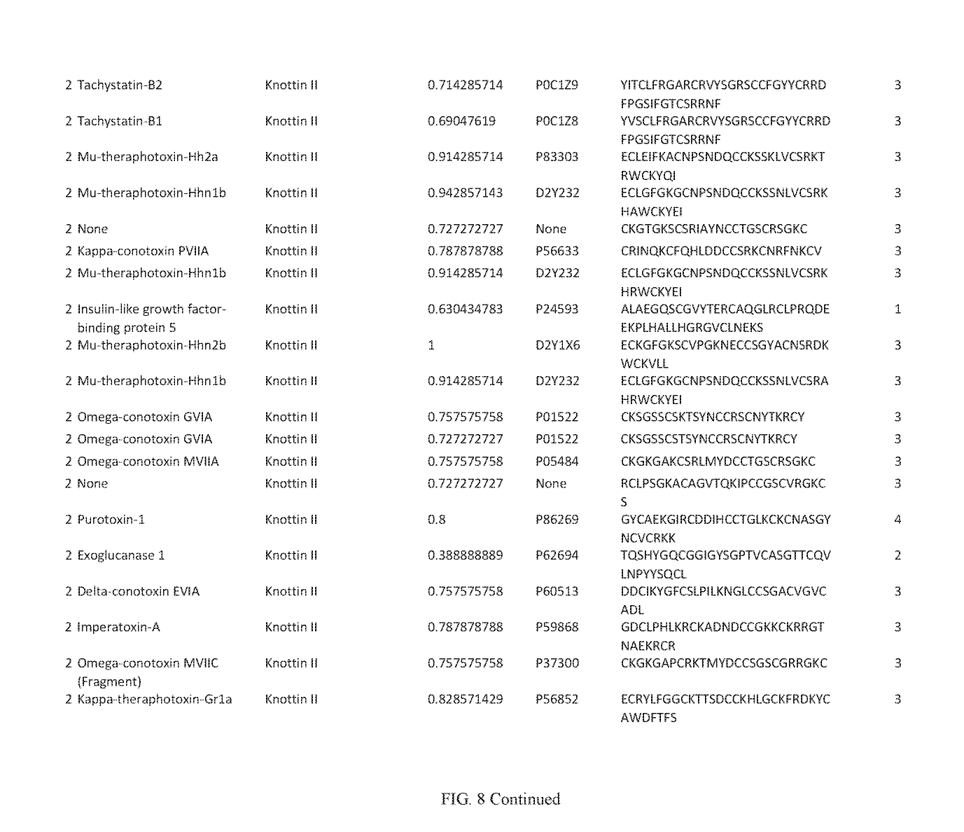

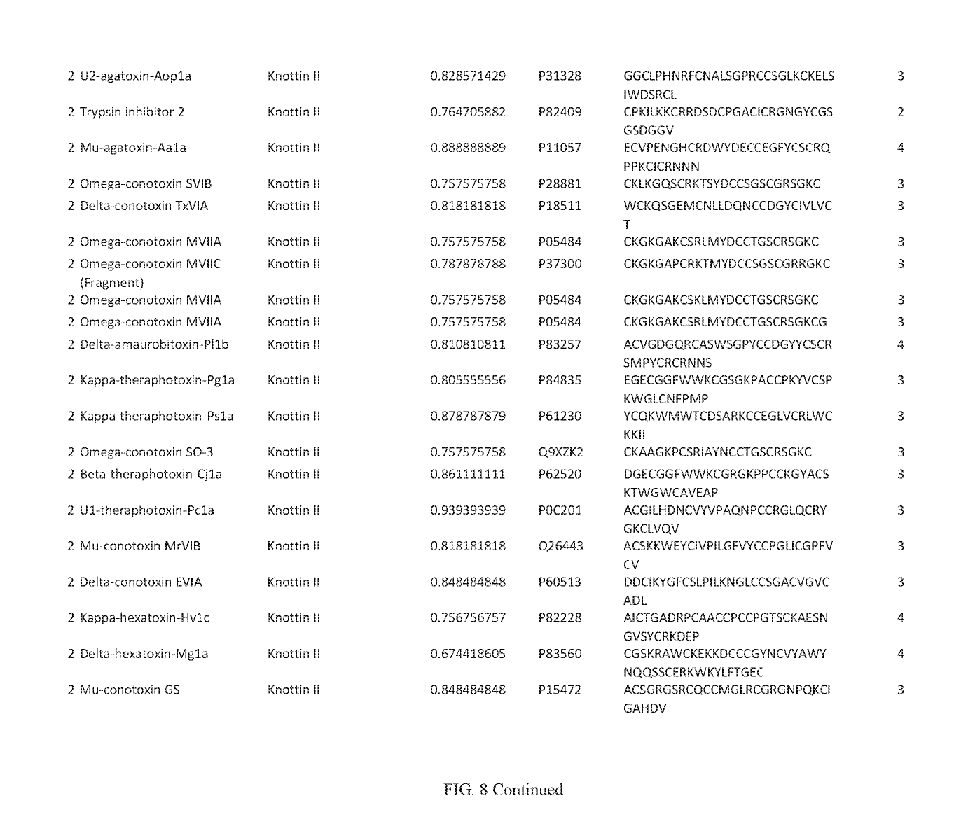

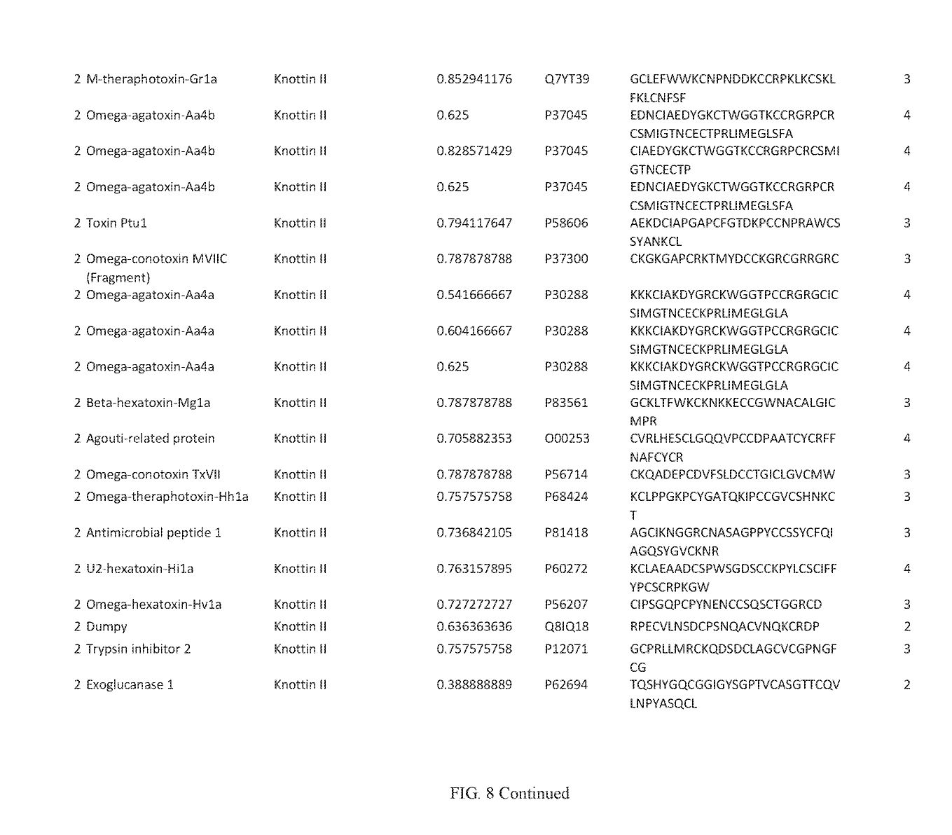

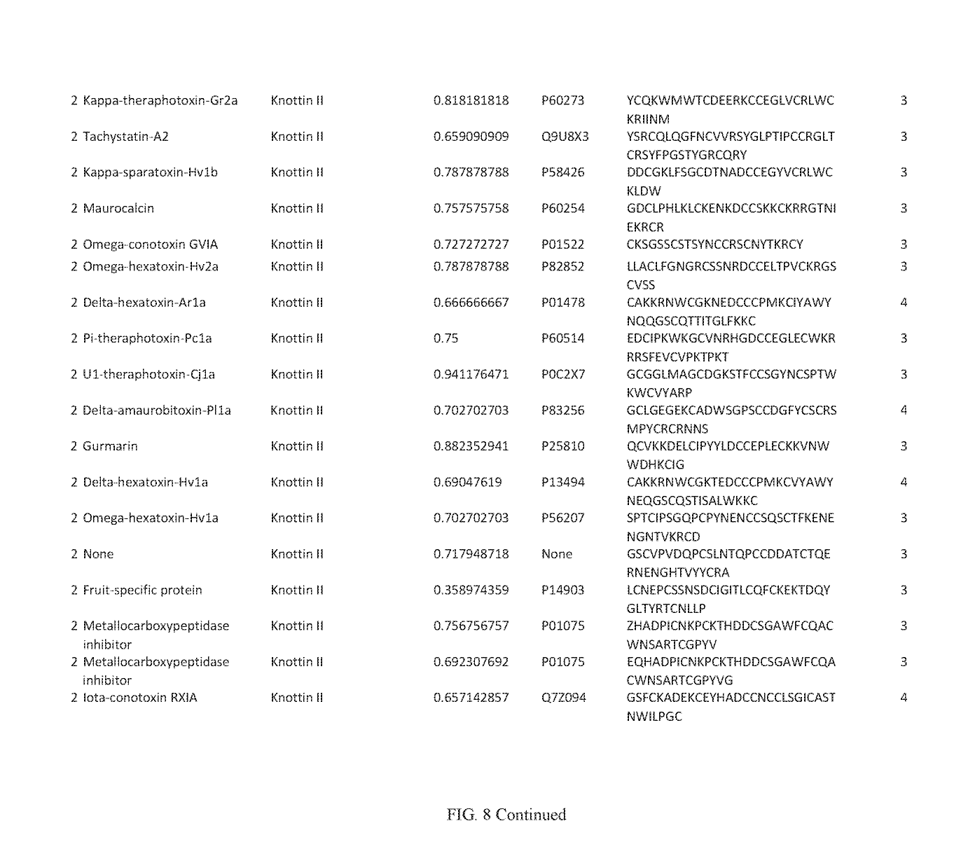

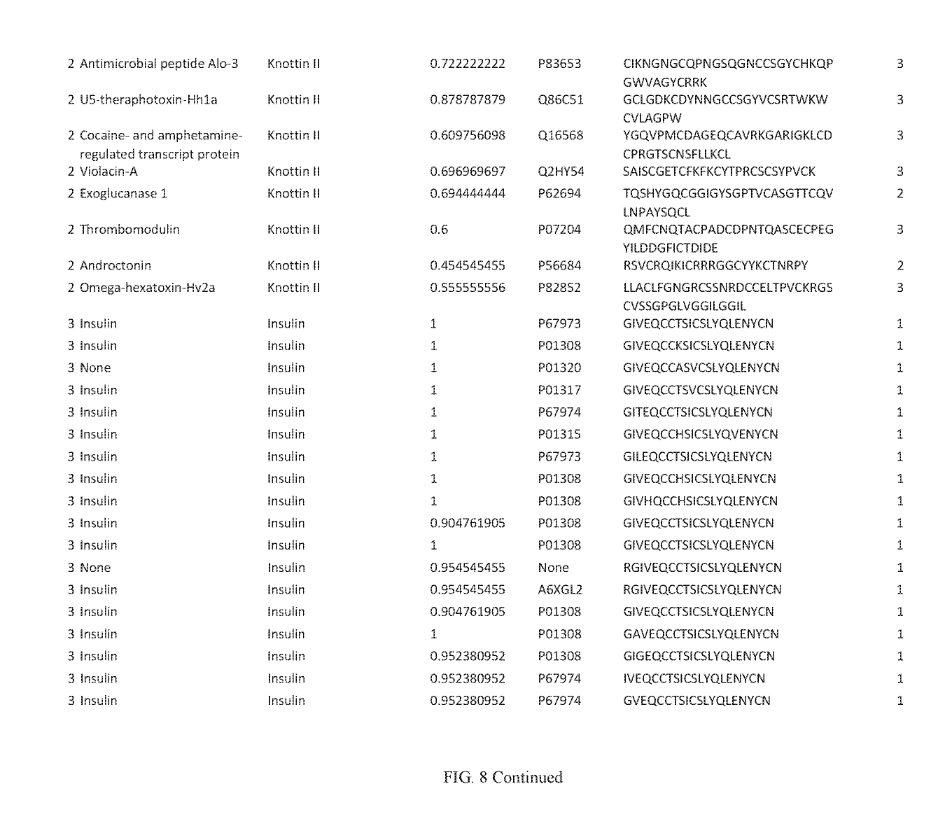

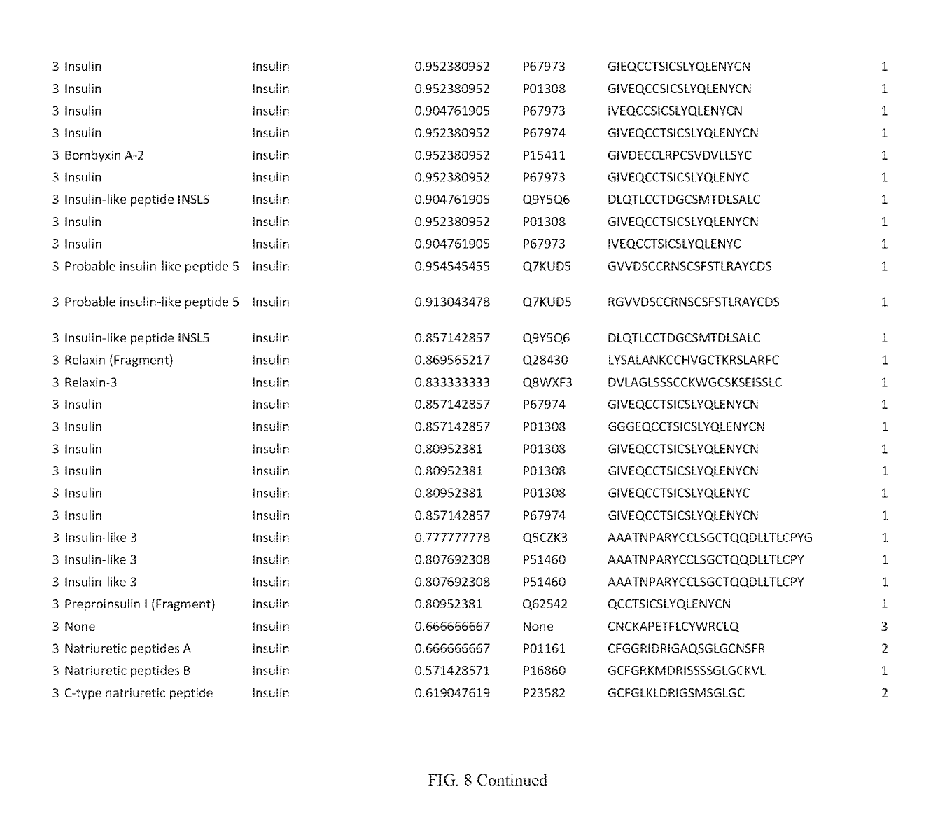

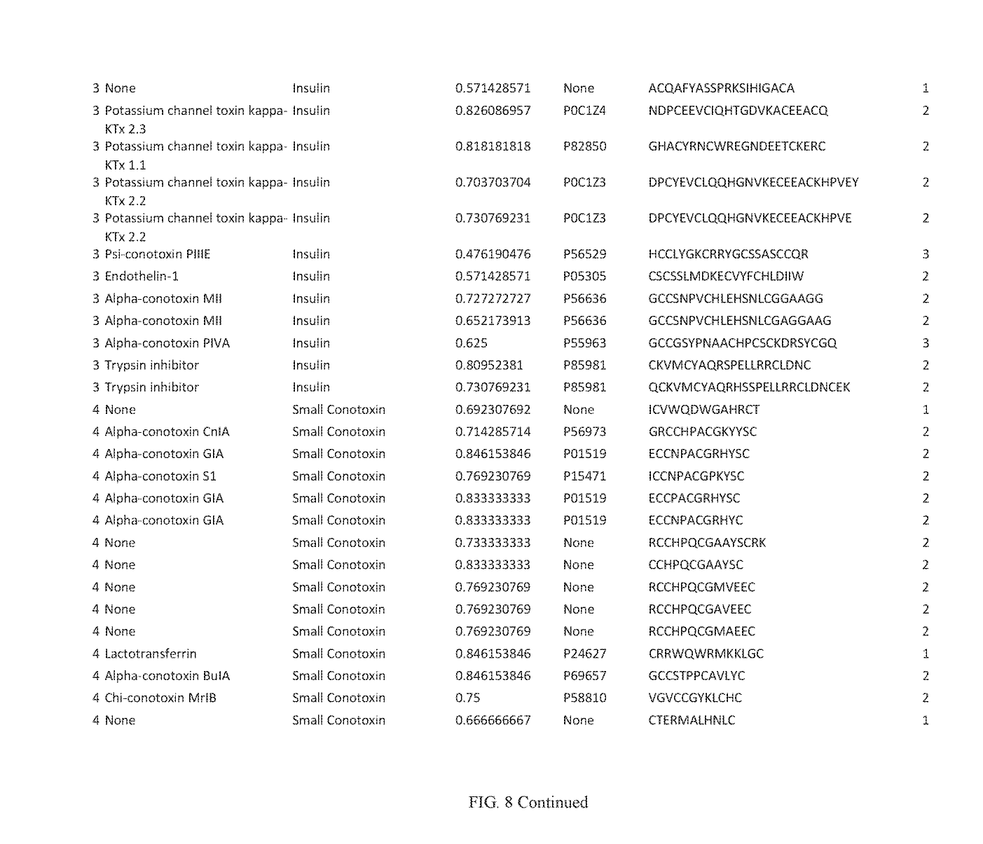

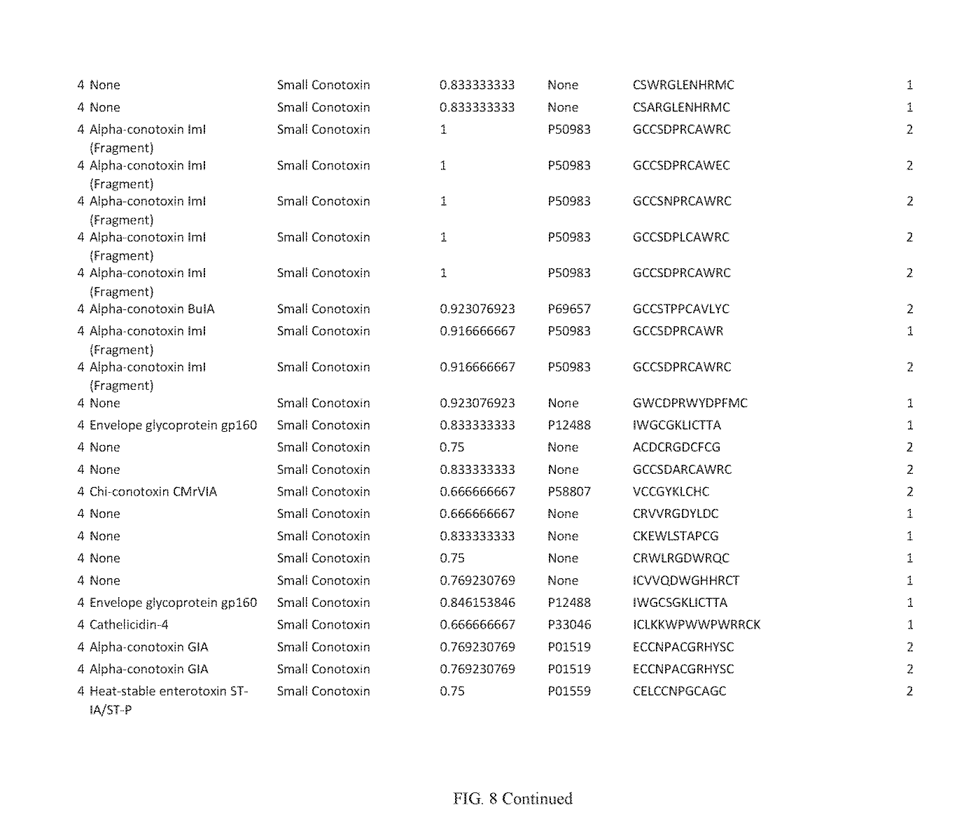

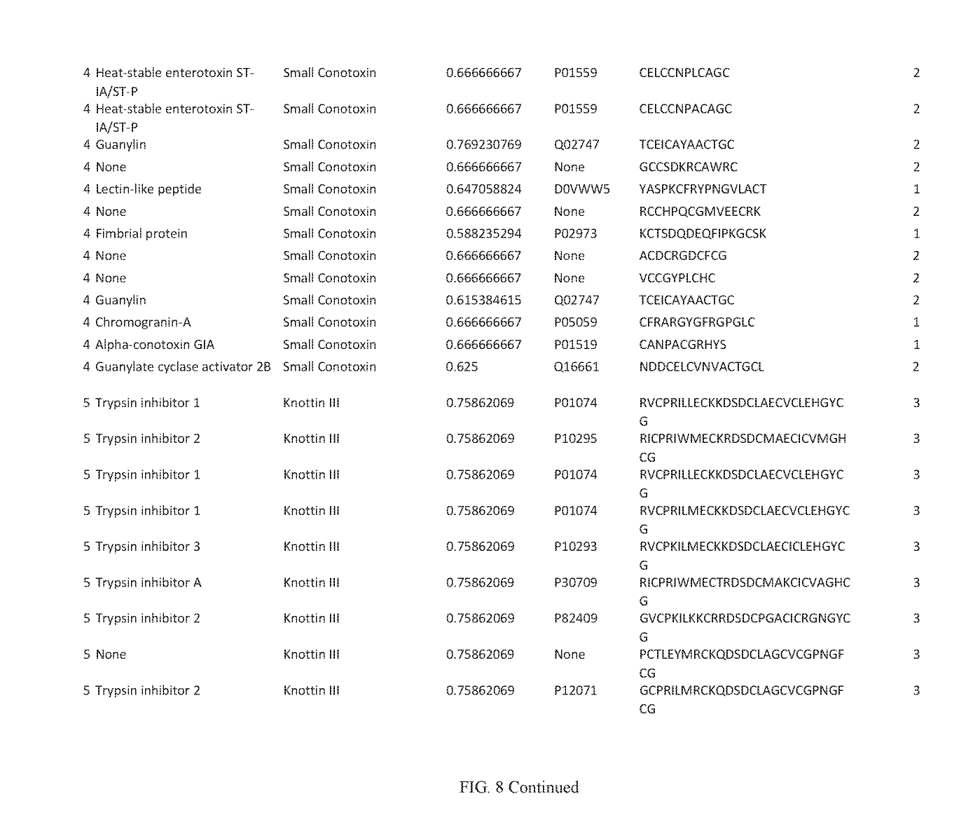

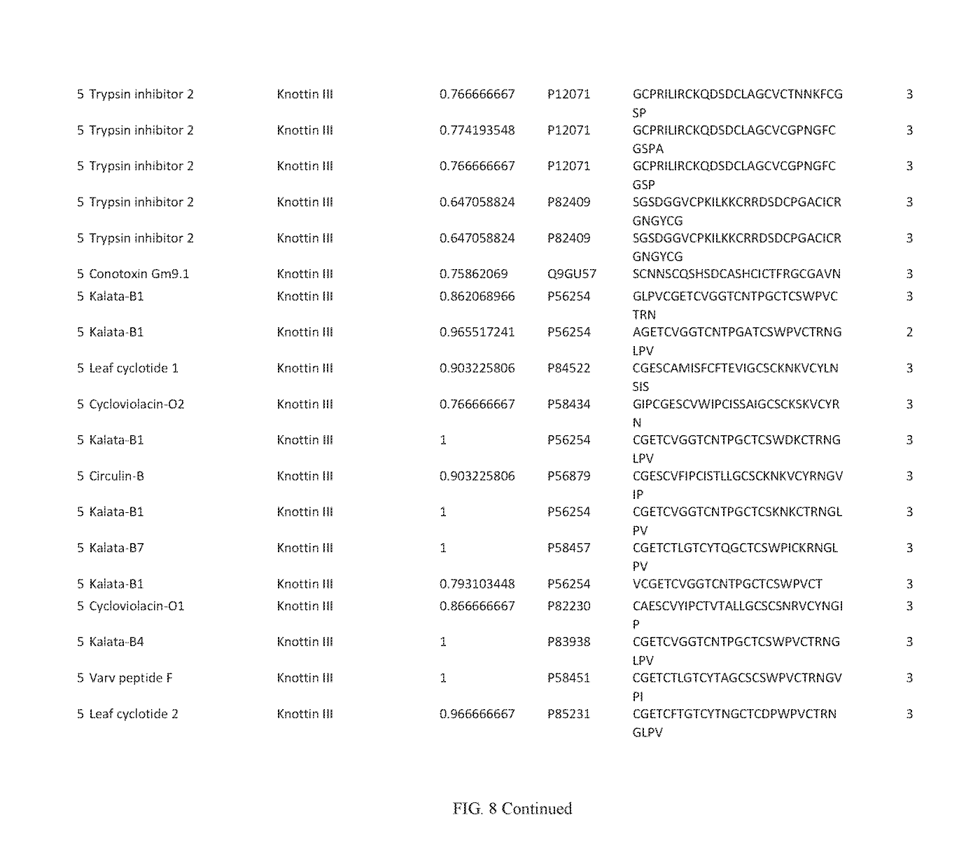

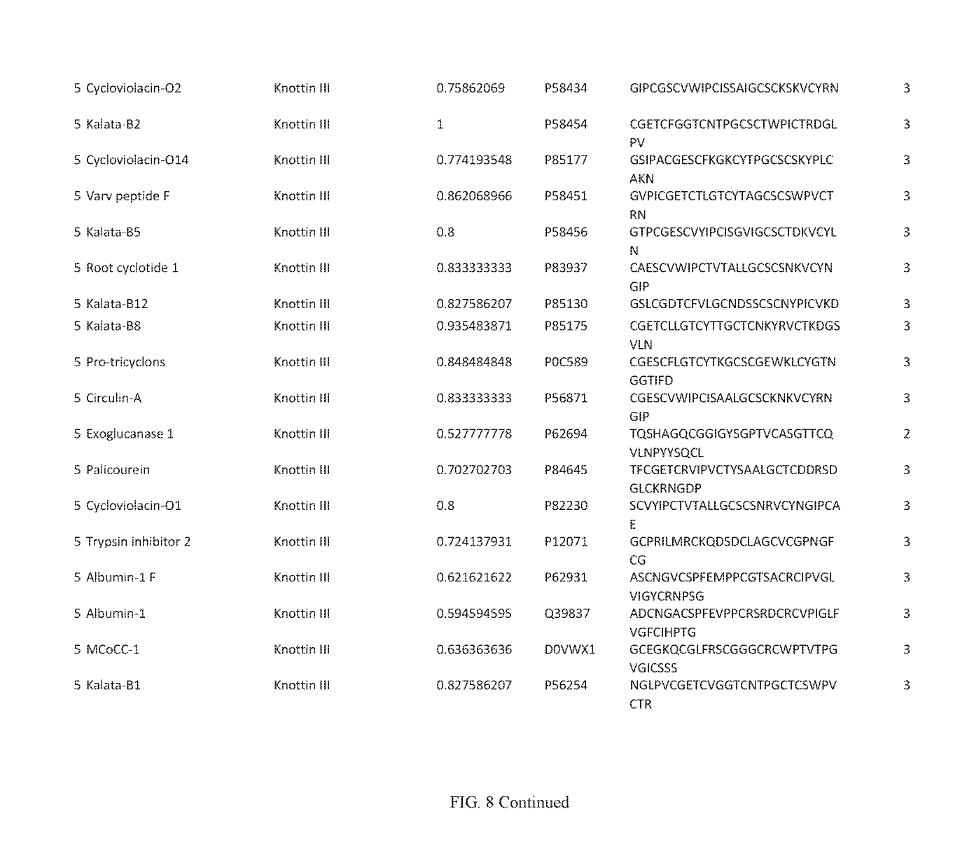

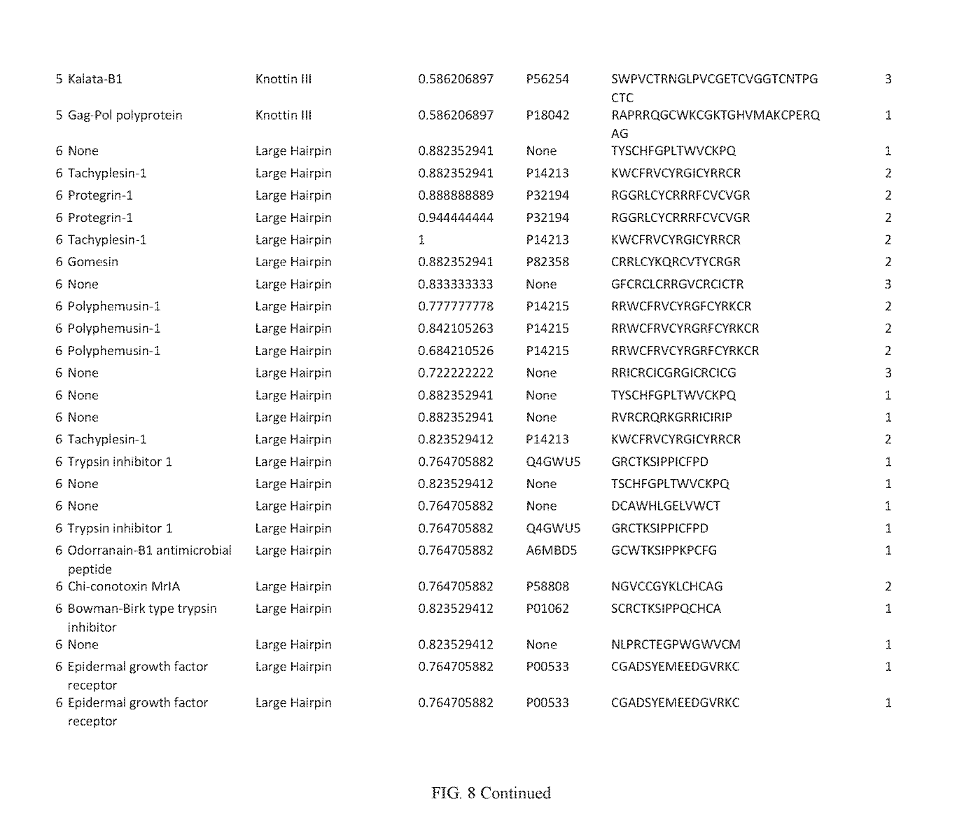

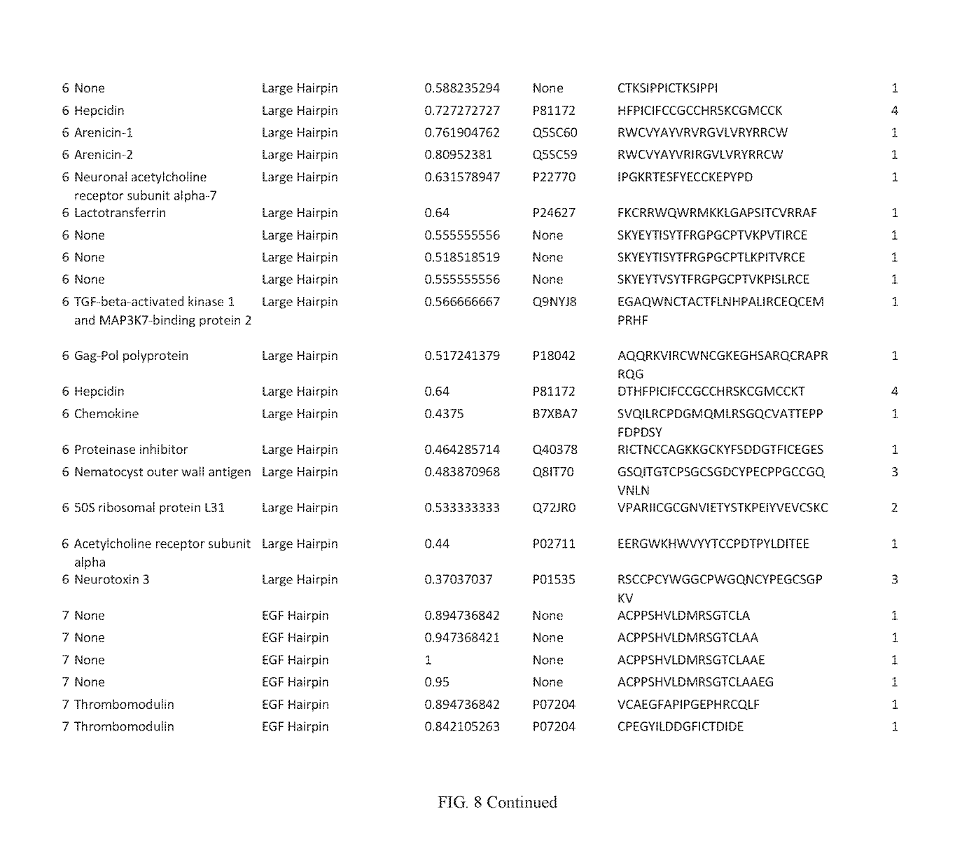

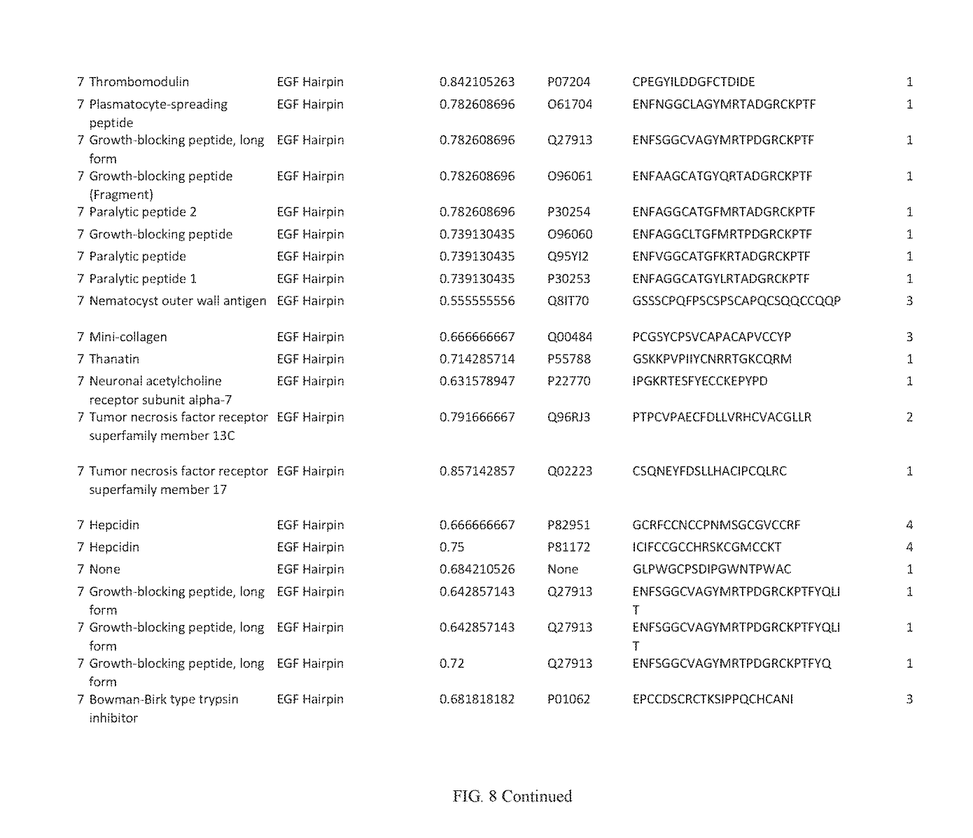

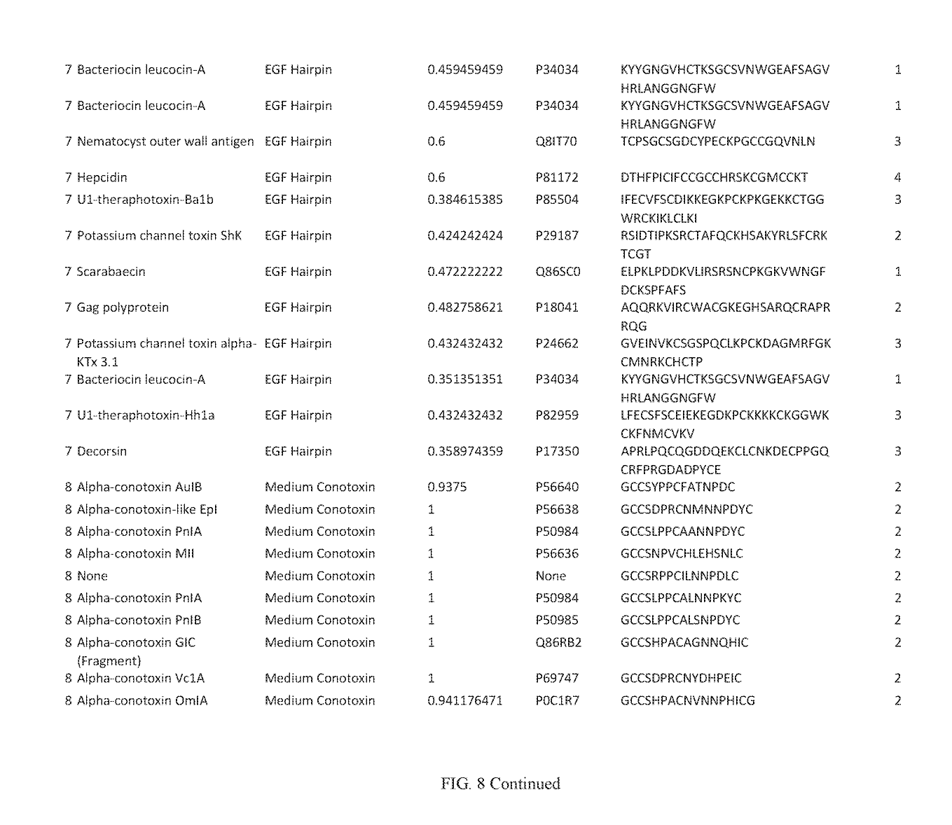

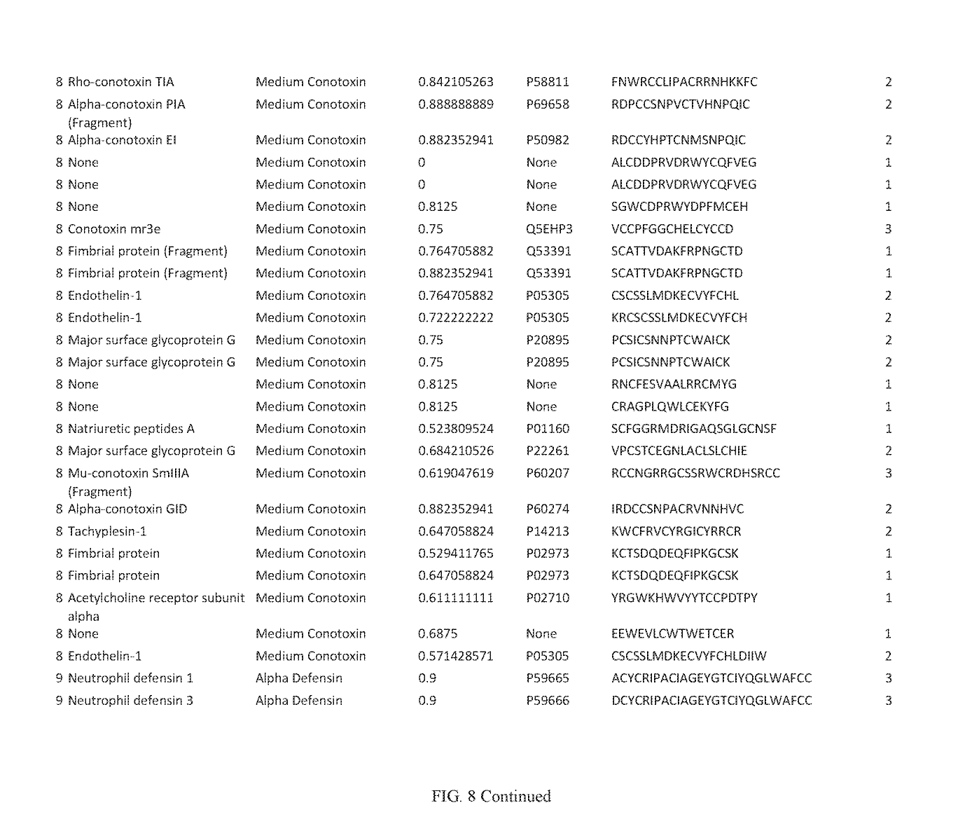

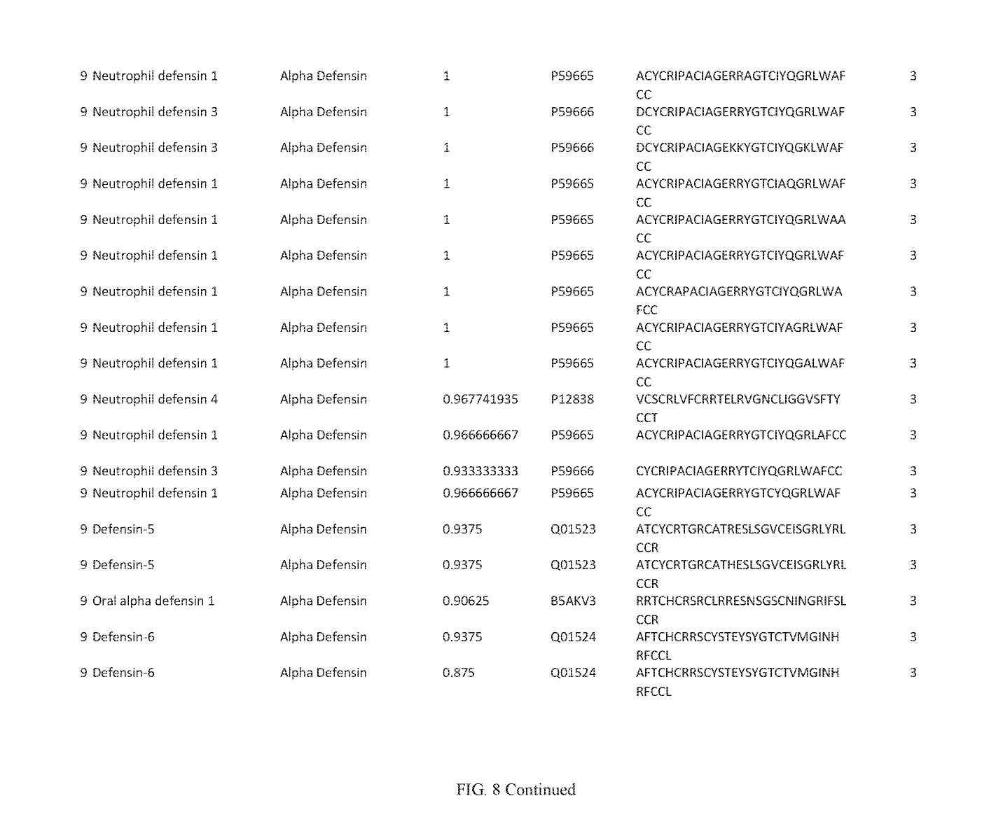

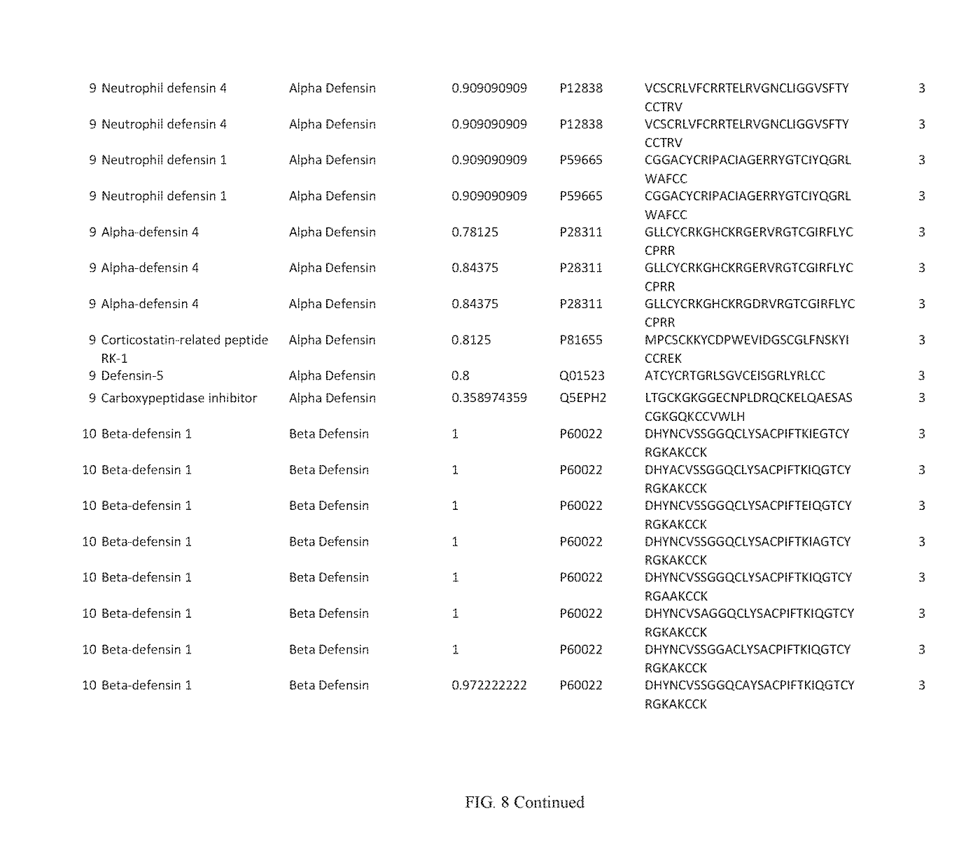

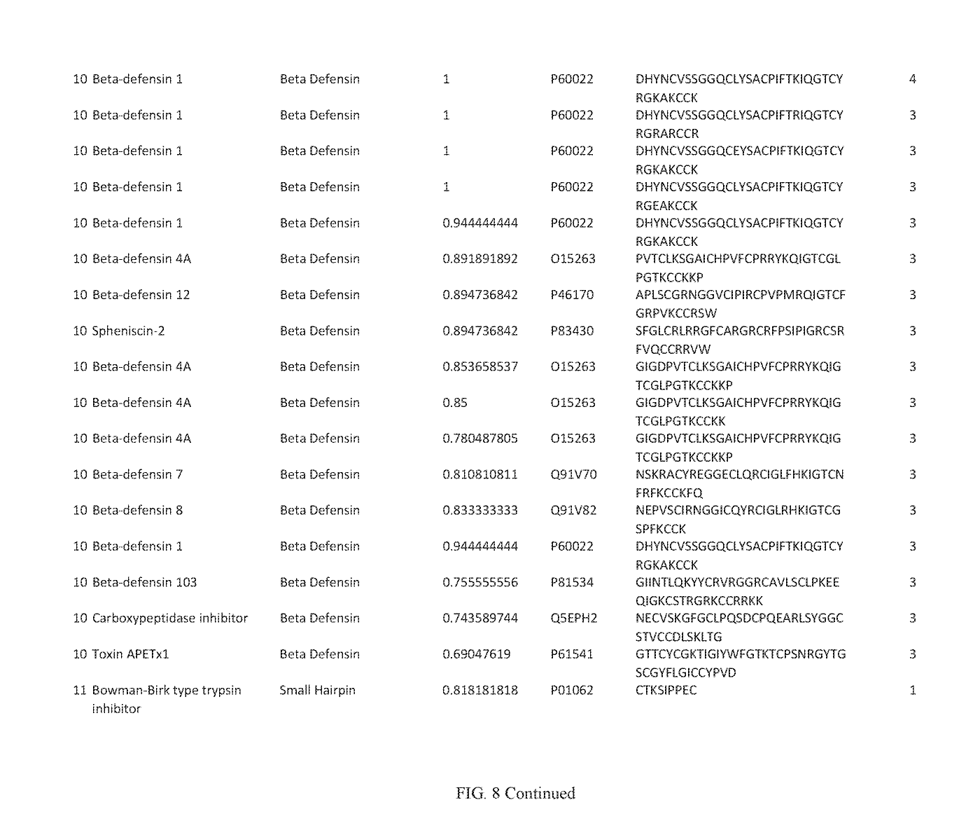

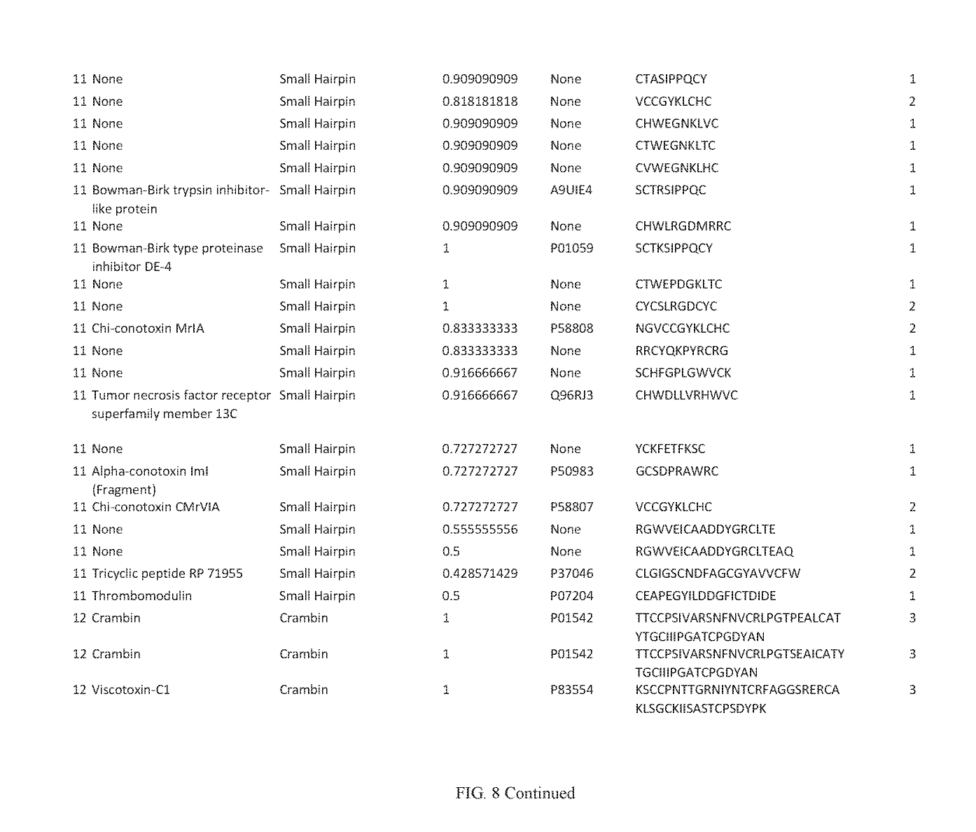

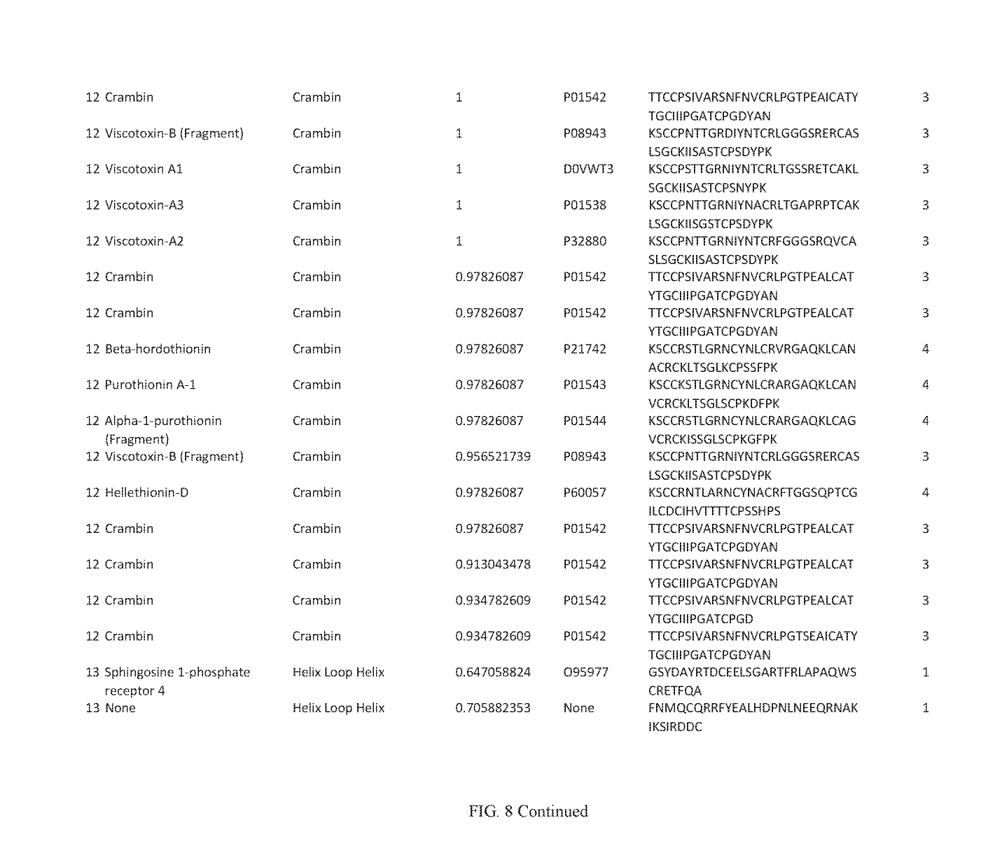

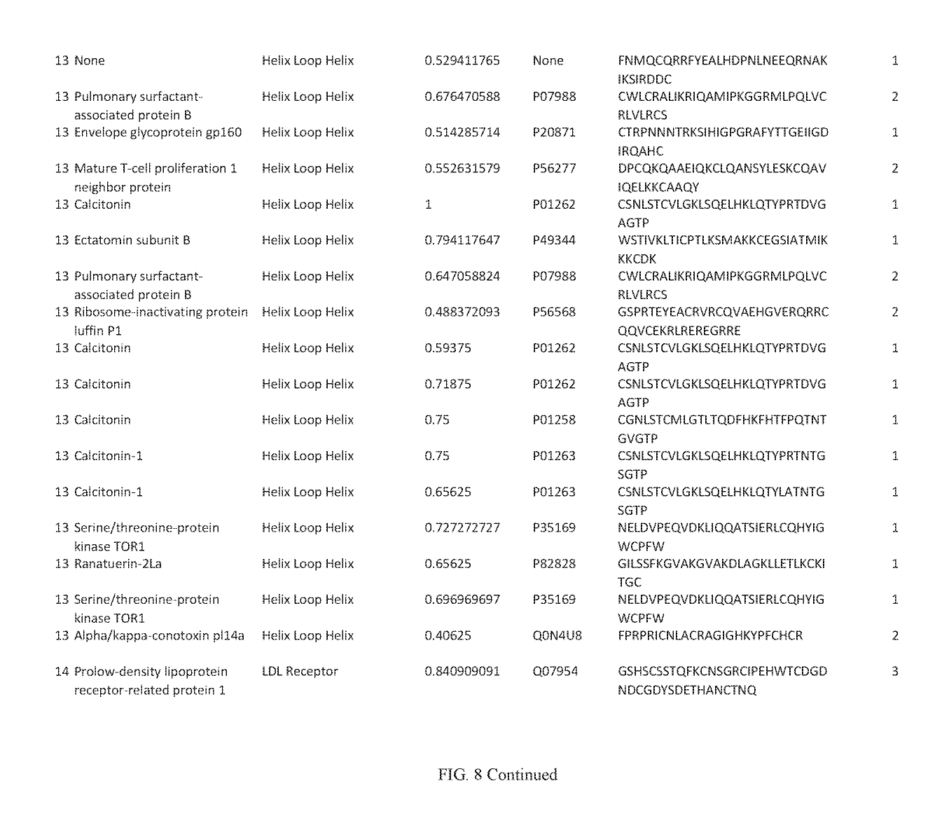

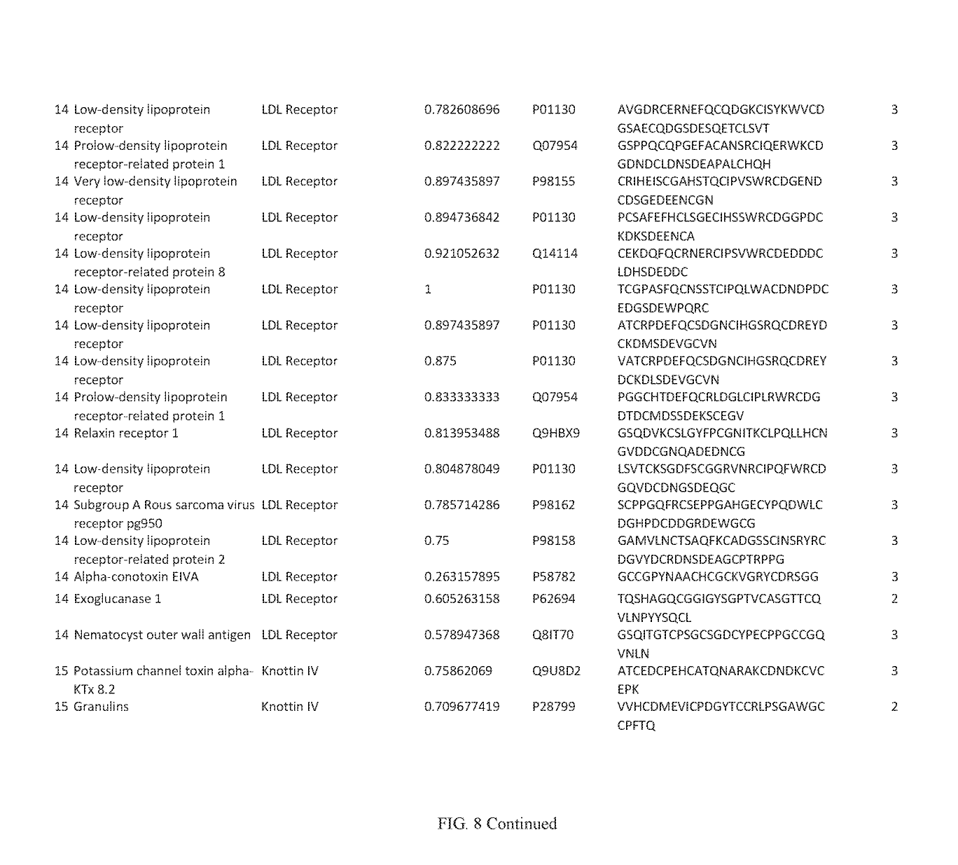

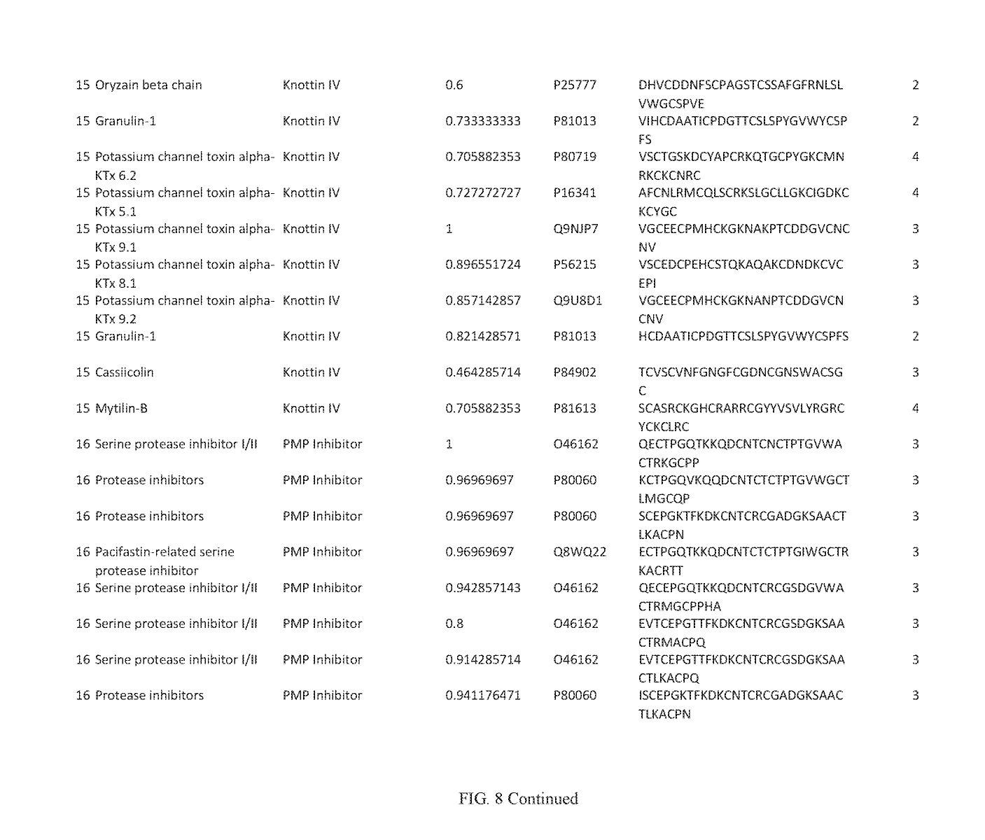

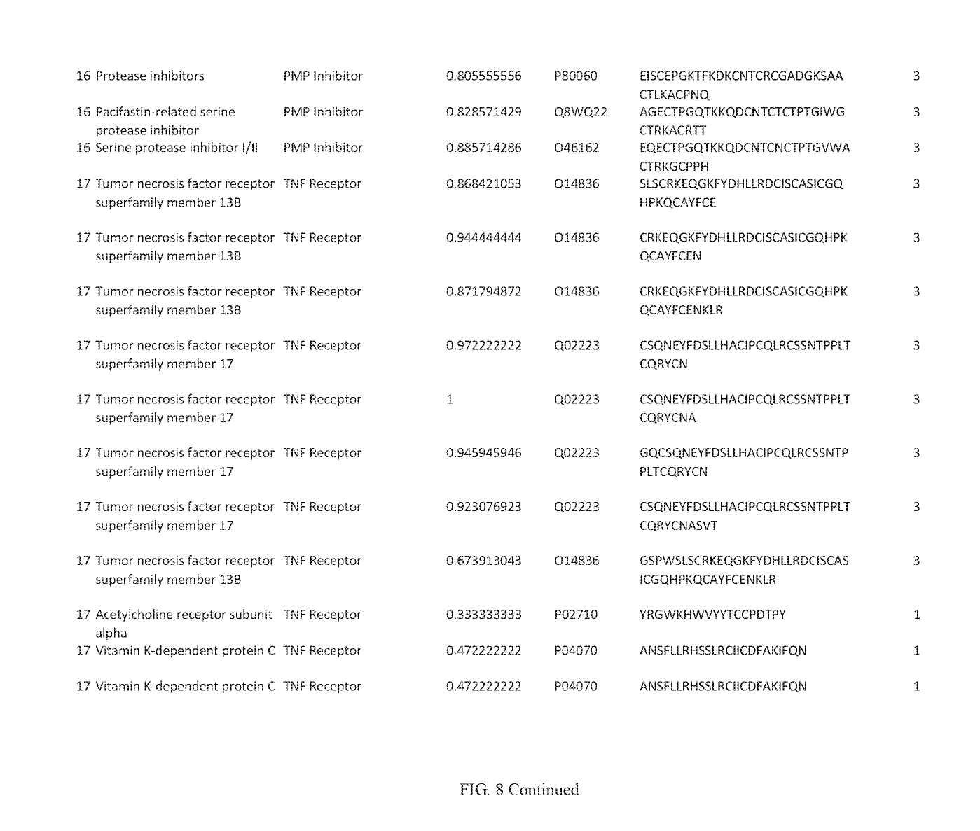

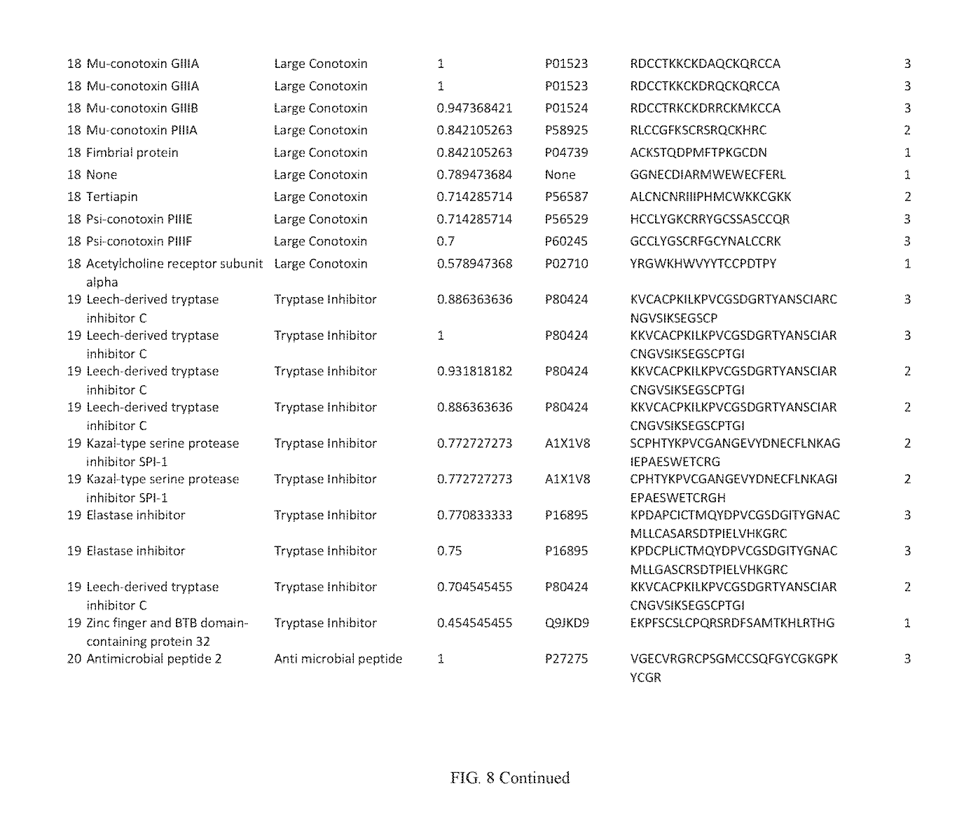

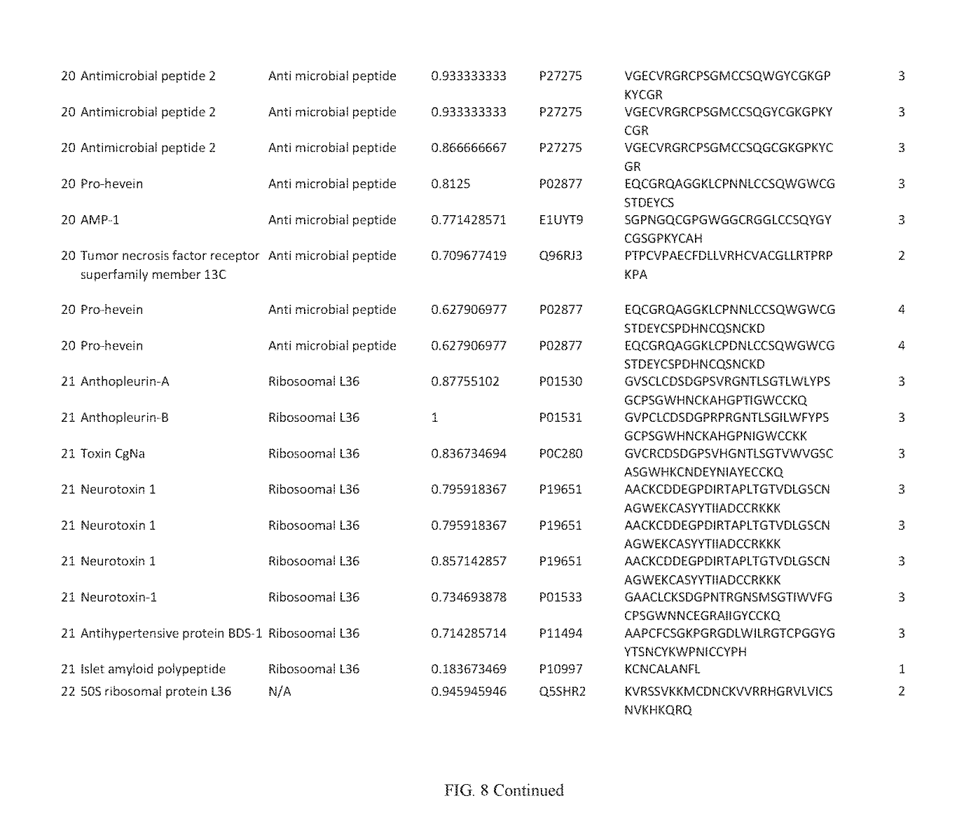

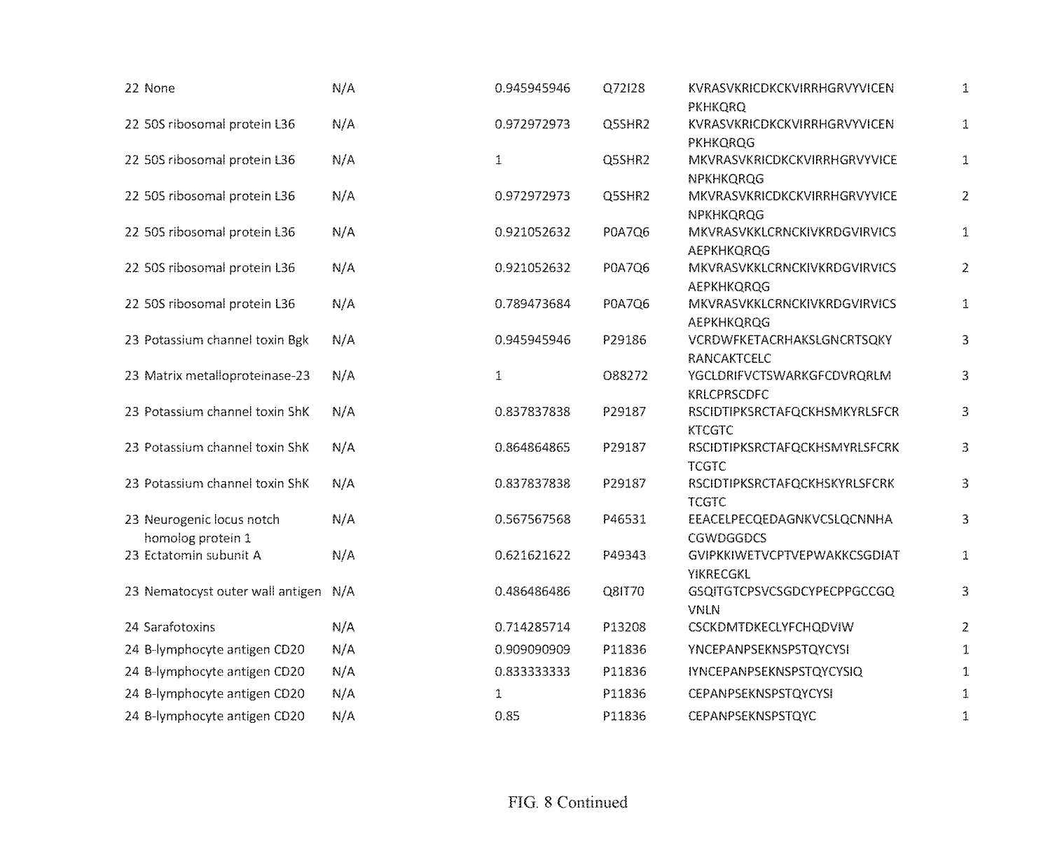

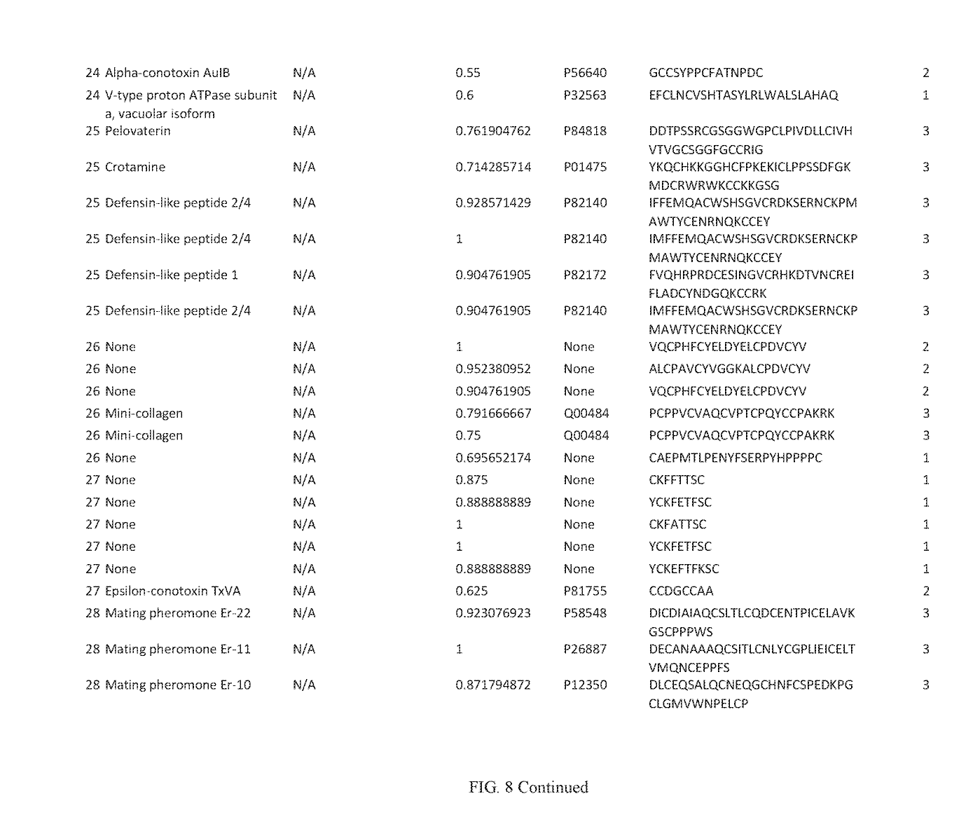

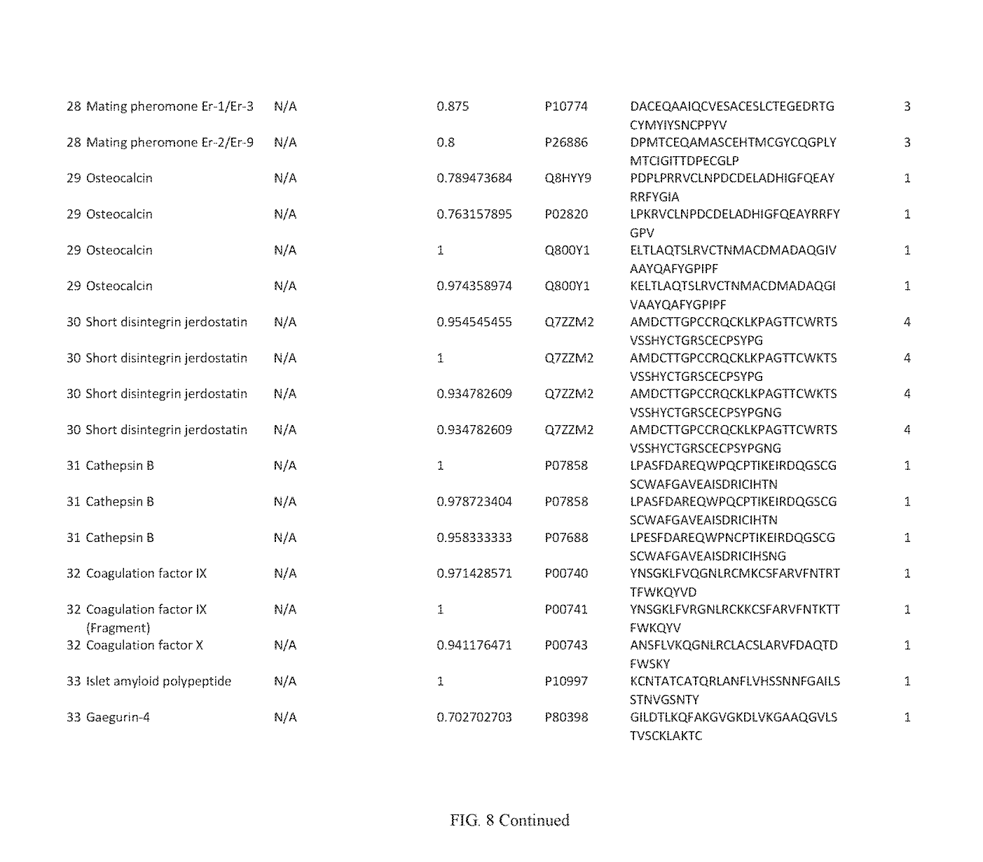

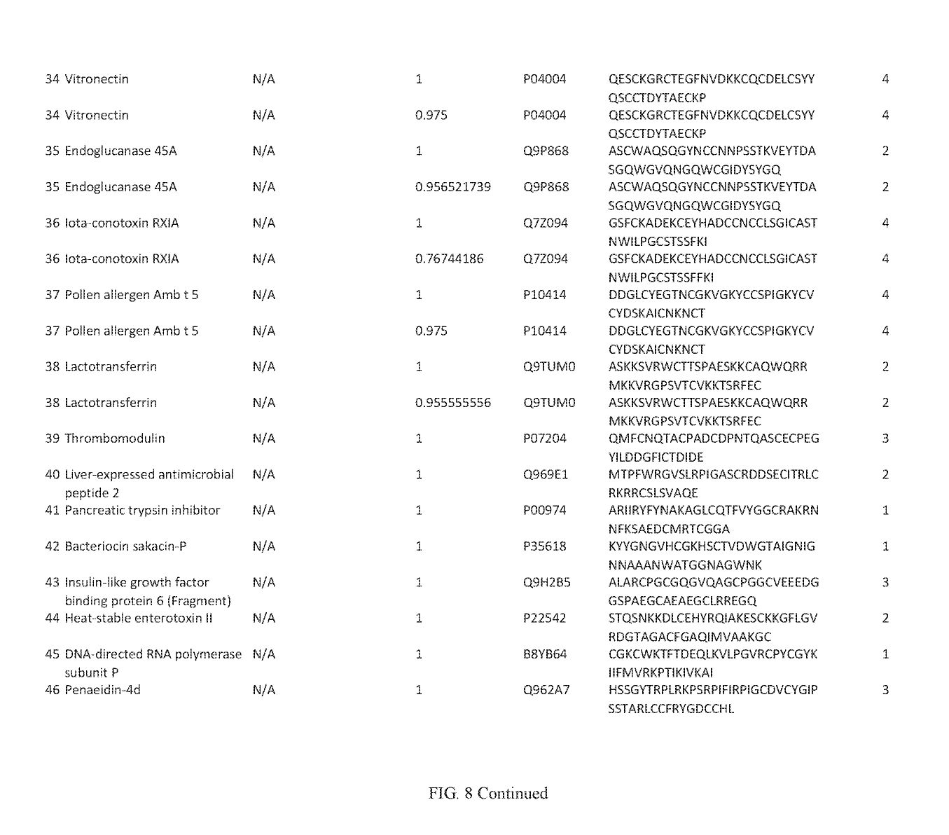

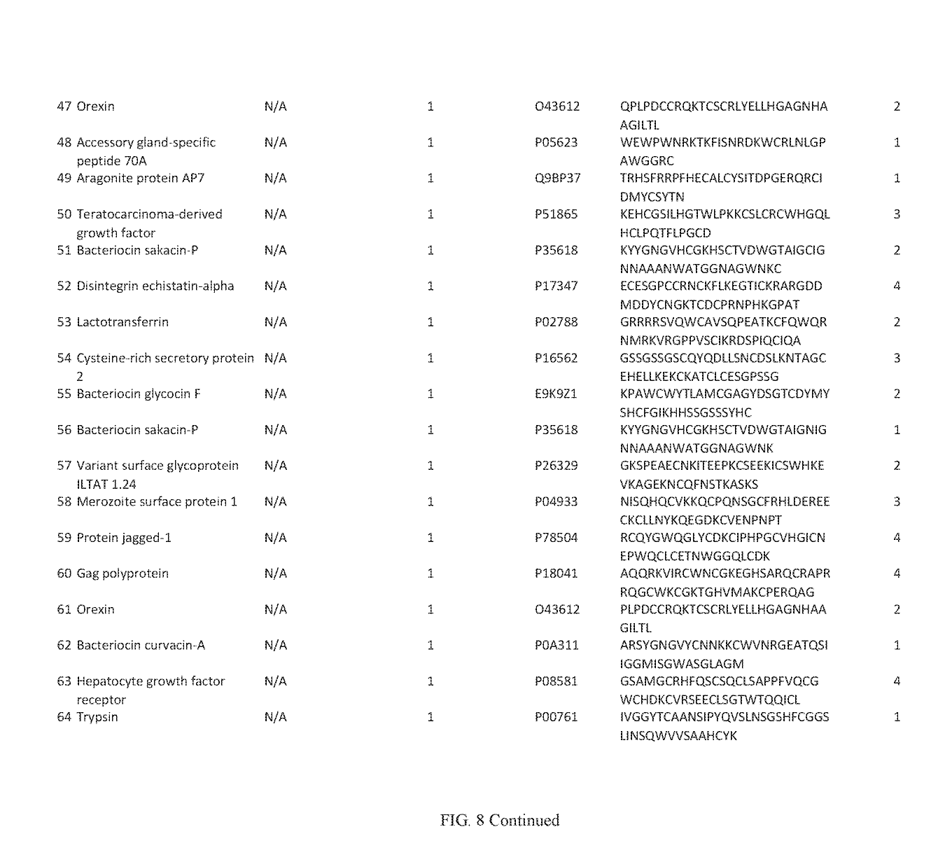

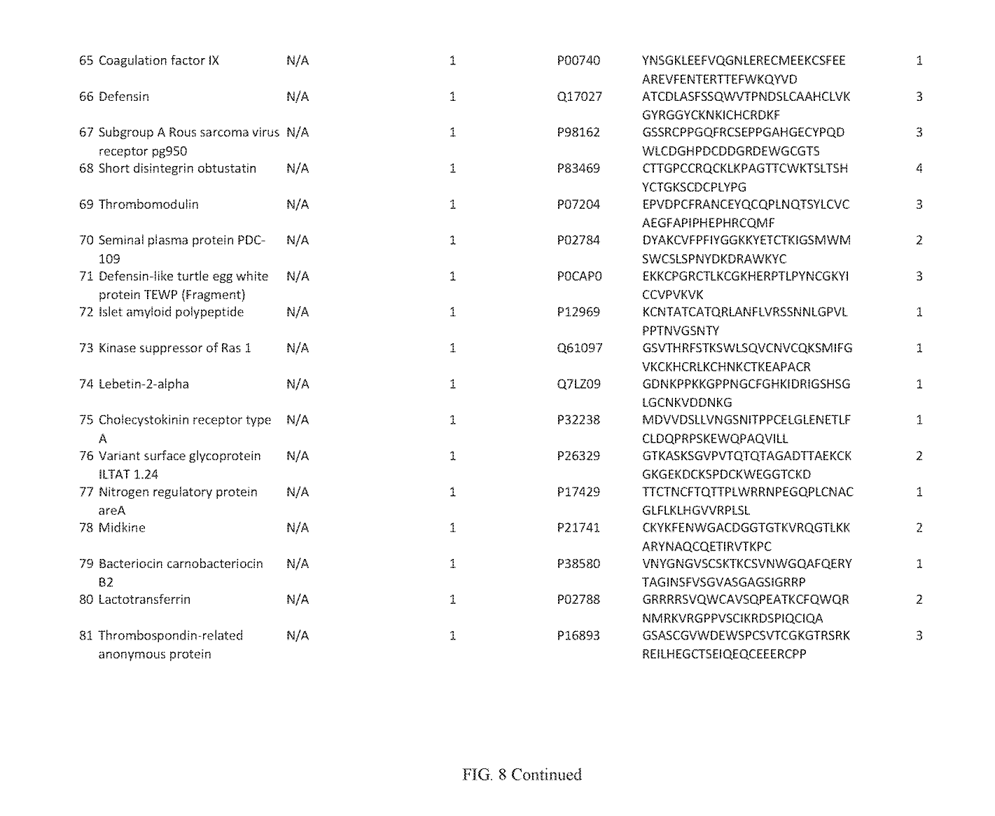

The tertiary structures associated with each of 20 DRP clusters and related libraries of the present invention are depicted in FIG. 4, and are described based on the presence of a dominant trait as: (1) knottin 1, (2) knottin 2, (3) insulin, (4) small conotoxin, (5) knottin 3, (6) small hairpin, (7) EGF-like hairpins, (8) medium conotoxin, (9) α-defensin, (10) β-defensin, (11) large hairpin, (12) crambin, (13) helix-loop-helix, (14) LDL receptor, (15) knottin IV, (16) PMP inhibitors, (17) TNF receptor, (18) large conotoxin, (19) tryptase inhibitor, and (20) anti-microbial peptide. This figure shows the overlapping predicted structure of DRPs within the DRP cluster except for singletons. Sequence conservation between the DRPs with each DRP scaffold library is indicated, with regions of high conservation shown in light gray, medium conservation shown in medium gray, and low conservation shown in dark gray. FIG. 8 provides a summary of peptides (DRPs) that fall within each of 20 different DRP clusters or scaffolds.

DRP Scaffold Libraries

The present invention also provides libraries of DRPs, including libraries based on any of the different DRP clusters described herein. In certain embodiments, the invention relates to diverse libraries of DRPs based on the identification of the unique set of DRP clusters. In particular embodiments, representative DRPs within each cluster form a DRP scaffold which is the basis for different DRP scaffold libraries of the present invention. Thus, in certain embodiments, a DRP library of the present invention comprises a plurality of DRPs generated by modification of a single representative DRP within a DRP cluster. Thus, members of a DRP library may comprise a common scaffold based on the representative DRP, e.g., the same disulfide bond pattern and one or more shared structural features of the representative DRP.

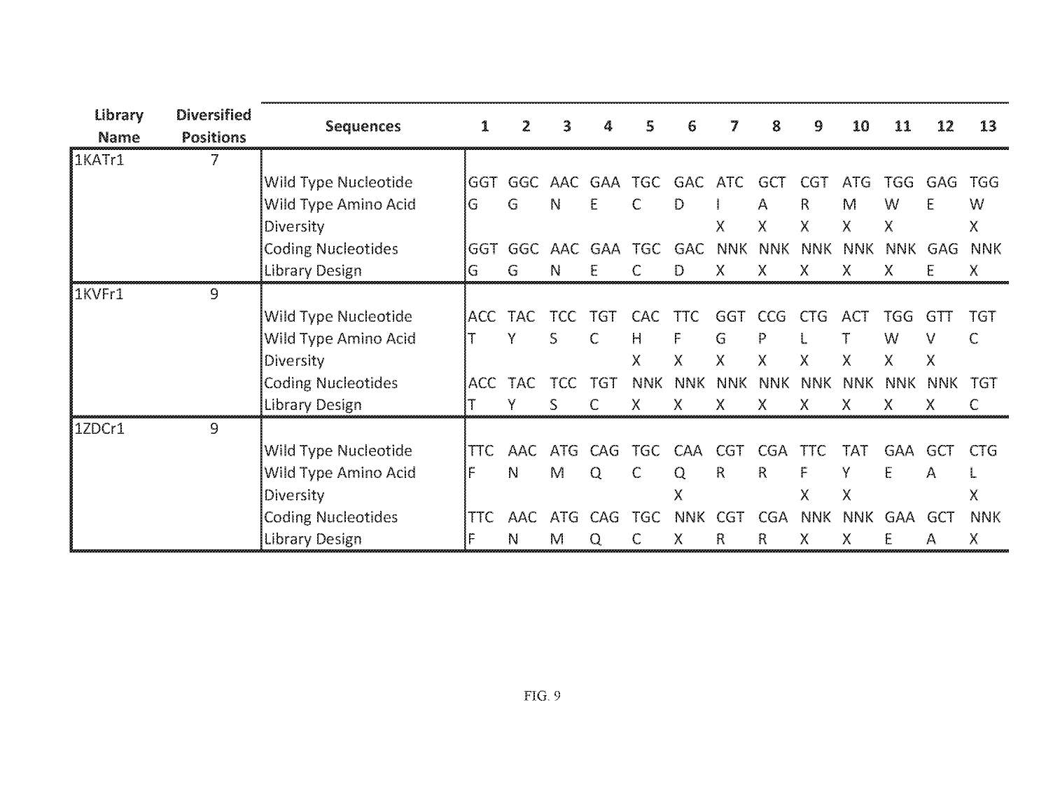

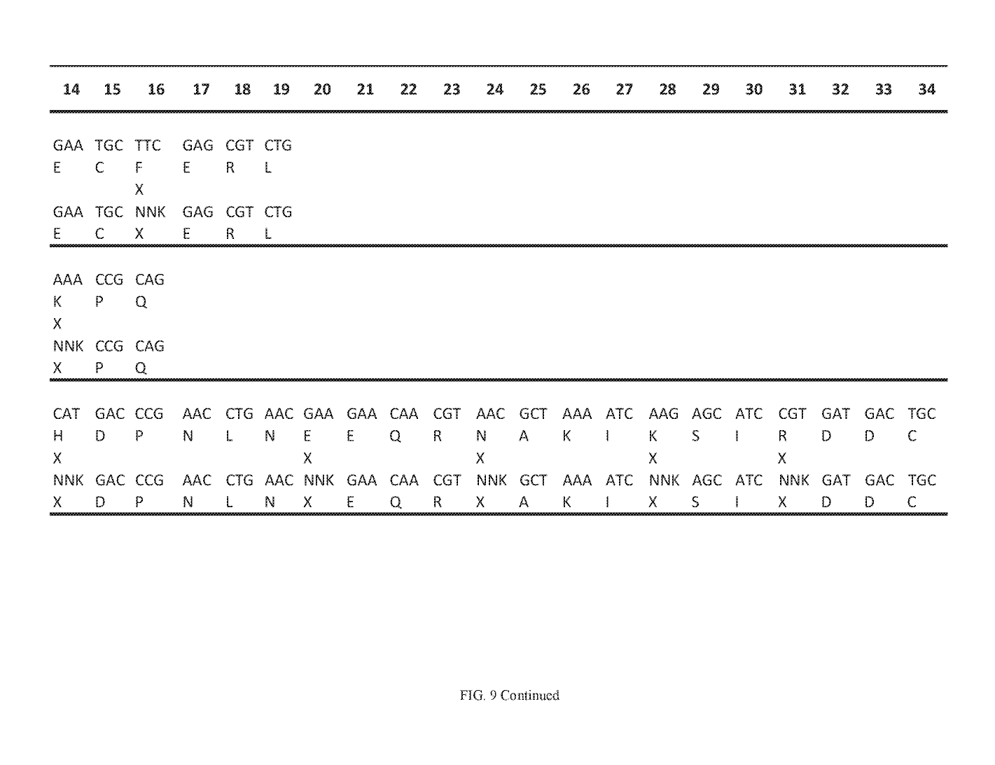

The present invention also includes representative DRPs within each DRP cluster described herein, which are used as the scaffold from which the DRP library is generated, e.g., by mutagenesis of certain amino acid residues within the representative DRP. In certain embodiments, the mutagenized amino acid residues do not include any of the cysteine residues that form disulfide bonds in the representative DRP. In certain embodiments, the mutagenized amino acid residues do not include all of the cysteine bonds forming disulfide bonds in the representative DRP, e.g., at least two cysteine residues that form a disulfide bond with each other are maintained. In certain embodiments, a representative DRP may be any DRP within a particular cluster described herein, and a DRP library may be prepared based on any such representative DRP. In particular embodiments, the representative DRP is the centroid DRP of a cluster. In particular embodiments, a DRP library comprises DRPs sharing a scaffold structure based on one or more representative DRPs within any of the clusters of DRPs described herein. In particular embodiments, a representative DRP is a DRP within the DRP cluster that has a certain level of sequence identity to the other DRPs within the same DRP cluster. In particular embodiments, a representative DRP has at least 30%, at least 40%, at least 50%, at least 60%, at least 70%, at least 80% amino acid identity to the other DRPs in the same DRP cluster. Representative DRPs for three clusters are shown in FIG. 9. This figure also shows amino acid residues (indicated by “X”) that may be substituted to generate a library of related DRPs.

The present invention includes libraries of DRPs, wherein a plurality of members of each library comprise one or more common structural feature with other members of the library. In particular embodiments, the DRPs within a library share the same disulfide bond pattern. Such a library may be referred to herein as a DRP scaffold library, since its members share a common scaffold structural feature. In particular embodiments, the common scaffold structural feature is one shared by the members of any of the 20 DRP clusters described herein. The present invention includes DRP scaffold libraries in which the DRPs within the scaffold library share a common DRP scaffold structural feature described in FIGS. 8 and 9 or depicted in FIG. 4. In particular embodiments, the present invention provides 20 different DRP scaffold libraries, each having a different DRP structural scaffold described in FIGS. 8 and 9 or depicted in FIG. 4. In particular embodiments, the members of a DRP scaffold library comprise amino acid sequence variants of a representative DRP within the corresponding DRP cluster, having one or more amino acid deletions, insertions or substitutions. In related embodiments, the present invention includes a system, kit or master library comprising a plurality of the 20 different DRP scaffold libraries, e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or all 20 of the DRP scaffold libraries described herein.

In certain embodiments, at least 80%, at least 90%, or all of the DRP peptides within a particular DRP scaffold library comprise or consist of an amino acid sequence having at least 20%, at least 30%, at least 40%, at least 50%, at least 60%, at least 70%, or at least 80% amino acid sequence identity to any one of the DRPs shown in FIG. 8. In certain embodiments, at least 80%, at least 90%, or all of the DRP peptides within a particular DRP scaffold library comprise or consist of an amino acid sequence having at least 30%, at least 40%, at least 50%, or at least 60% amino acid sequence identity to any one of the DRPs shown in FIG. 8. In particular embodiments, at least 80%, at least 90%, or all of the DRPs within a particular DRP scaffold library have at least 80% or at least 90% amino acid sequence identity to any one of the DRPs shown in FIG. 8 or FIG. 9, wherein X indicates any amino acid. In particular embodiments, at least 80%, at least 90%, or all of the DRPs within a particular DRP scaffold library have a sequence shown in FIG. 8 or FIG. 9, wherein X indicates any amino acid. In one embodiment, all of the DRPs within a particular DRP scaffold library comprise any one of the amino acid sequences shown in FIG. 8 or FIG. 9.

In particular embodiments, all or substantially all of the DRPs within a DRP scaffold library are variants of a representative DRP within the DRP scaffold. In certain embodiments, a plurality of the DRP peptides of a DRP scaffold library are variants of a single DRP peptide that falls within the particular DRP cluster, i.e., a representative DRP. For example, the plurality of peptides within a DRP scaffold library may comprise one or more amino acid modifications, e.g., insertions, deletions or substitutions, as compared to the representative DRP. In particular embodiments, the representative DRP has a sequence shown in FIG. 8 or FIG. 9. In particular embodiments, the one of more amino acid modifications fall within an amino acid indicated as X in any one of the representative DRP sequences shown in FIG. 9.

The DRPs within the DRP scaffold library may comprise one or more amino acid modifications as compared to the representative DRP sequence, e.g., one or more amino acid insertions, deletions or replacements. In certain embodiments, at least some of the amino acid modifications are present in regions or domains on an exposed or surface of the peptide, e.g., to allow binding to a polypeptide or other target of interest. In certain embodiments, the representative peptide sequence comprises or consists of a sequence shown in FIG. 8 or FIG. 9, and, in particular embodiments, the amino acid modifications present in the DRPs present within the DRP scaffold library based on the consensus peptide sequence shown in FIG. 9 comprise one or more amino acid modifications of the corresponding amino acid residues indicated by X in FIG. 9.

While any of the DRPs within a cluster may serve as the representative DRP sequence upon which a DRP library scaffold is based, in certain embodiments, the representative DRP is selected as described herein (see, e.g., Example 1). In particular embodiments, the representative DRP within a cluster that is used to produce a DRP scaffold library for that cluster is selected based on one or more of the following criteria: (i) how close the DRP is to the centroid of the cluster; (2) the amino acid length of the DRP; (3) the number of disulfide bonds in the DRP; (4) any reported stability data on the DRP, including oral stability; (5) whether the DRP has previously been used to produce libraries, such as phage display libraries. One of skill in the art could readily determine an appropriate DRP within a cluster to serve as the basis for generating a DRP library. In particular embodiments, the DRP is selected due to being close to the centroid of the cluster, having a short amino acid length (e.g., 10 to 50 amino acids or 11 to 49 amino acids), having few disulfide bonds (e.g., less than four, less than three, two or one), evidence of stability, such as oral stability, and evidence of being compatible for use in phage display, and/or ease of synthesis of the DRP. In certain embodiments, the representative DRP is the centroid DRP, which may be determined as described herein. In particular embodiments, the representative DRP is selected for being, as compared to other DRPs in the cluster, more flexible, more functional (in a complex); smaller, experimental biased (on phage display); tissue bias (e.g., isolated from gastrointestinal tract or other tissue of interest), most promiscuous, or least promiscuous. In certain embodiments, the representative DRP is selected based on having a diverse set of structural features,

In particular embodiments, a DRP scaffold library is produced by generating a library of DRP peptide variants of the selected representative DRP sequence within a particular DRP cluster. In particular embodiments, the variants comprise one or more amino acid substitutions, deletions or insertions within one or more “contact” surface identified in the core or consensus DRP. In particular embodiments, the contact surface interacts with another protein or other molecule such as a nucleic acid, nucleotide or nucleoside (e.g. ATP or GTP) carbohydrate, glycoprotein, lipid, glycolipid or small organic molecule (e.g. a drug or toxin) without limitation thereto. In particular embodiments, the contact surface is a binding domain, e.g., a ligand-binding domain of a receptor, a receptor-binding domain of a ligand, a DNA-binding domain of a transcription factor, an ATP-binding domain of a protein kinase, chaperonin or other protein folding and/or translocation enzyme, a receptor dimerization domain or other protein interaction domains such as SH2, SH3 and PDB domains, or a domain that binds a small organic molecule or other molecule, although the skilled person will appreciate that the present invention is not limited to these particular examples. In particular embodiments, the contact surface comprises a structural feature selected from loops, β-turns or other contact surfaces, helical regions, extended regions and other protein domains. In particular embodiments, the contact surface is exposed to solvent when in solution. In certain embodiments, the surface region of the DRP to modify for generating a diverse library is selected based on: (1) the flexibility of the surface; (2) the diversity of amino acids found on the surface when taking the cluster as a whole; the size of the surface and the combination of secondary structure within the surface. The skilled artisan could determine surfaces to modify based on these characteristics. In particular embodiments, the mutated region of the DRP is selected to be flexible, promiscuous (diverse in amino acids), large, and/or unique (includes a combination of secondary features).

Contact surfaces may be identified in DRPs described herein using methods available in the art, including, e.g., structural modeling or determined crystal structure, selecting residues that form a binding surface that is either predicted or experimentally defined, with high solvent accessibility, or surface patches with different secondary structures. For example, prediction of protein binding sites in protein structures using hidden Markov support vector machine is described in Liu, B. et al., BMC Bioinformatics, 2009, 10:381. Prediction of protein interaction sites from sequence profile and residue neighbor list is described in Hou, X. Z. et al., Proteins, 44: 336-343. In particular embodiments, structural features and different putative contact surfaces are identified as described herein (see, e.g., Example 1), or as described in U.S. Pat. No. 8,635,027 or 7,092,825, both of which are hereby incorporated by reference in their entirety.

In particular embodiments, a DRP scaffold library may include at least 1×10⁵, at least 1×10⁶, at least 1×10⁷, at least 1×10, at least 1×10⁹, or at least 1×10¹⁰ different DRP peptides. In particular embodiments, at least 80%, at least 90%, a plurality of, or all peptides within a particular DRP scaffold library share at least 30%, at least 40%, at least 50%, at least 60%, at least 70%, at least 80%, at least 90%, or at least 95% sequence identity to a consensus or core DRP, e.g., a consensus DRP sequence set forth in FIG. 8 or FIG. 9. In particular embodiments, at least 80%, at least 90%, at least 95%, at least 98%, at least 99%, or all peptides within a particular DRP scaffold library comprises or consist of any one sequence set forth in FIG. 8 or FIG. 9, wherein X indicates any amino acid.

In particular embodiments, the one or more amino acid modification of the core or consensus DRP occur within one or more defined region of the representative DRP. In particular embodiments, this region does not affect one or more of the shared scaffold structural features of the particular DRP scaffold peptides within the particular DRP scaffold library. In particular embodiments, the one or more amino acid modification does not alter the cysteine residues of the DRP peptides or does not alter the cysteine residues that participate in disulfide bonds within the DRP peptides. In particular embodiments, the one of more amino acid modifications fall within an amino acid indicated as X in any one of the consensus DRP sequences shown in FIG. 9. In particular embodiments, DRP scaffold libraries based on any of the consensus sequences shown in FIG. 8 or FIG. 9 retain at least 80%, at least 90%, or at least 95% of the indicated amino acid residues, wherein X may be any amino acid residue.

In particular embodiments, all or substantially all of the DRPs within a DRP scaffold library share a common three-dimensional polypeptide structural feature, e.g., a polypeptide surface feature or a core feature. In certain embodiments, the common three-dimensional polypeptide structural feature is based on structural similarity and/or disulfide bond conservation. In particular embodiments, the disulfide bond conservation is a distance between disulfide bonds of about 1.5 Å to about 2.5 Å. In particular embodiments, the distance between disulfide bonds is about 2.0 Å. In other embodiments, the common three-dimensional polypeptide structural feature of each DRP scaffold library is depicted in FIG. 4. In particular embodiments, is characterized as or is shared by one of the following polypeptide groups: knottin 1, knottin 2, insulin, small conotoxin, knottin 3, small hairpin, EGF-like hairpins, medium conotoxin, α-defensin, β-defensin, large hairpin, crambin, helix-loop-helix, LDL receptor, knottin IV, PMP inhibitors, TNF receptor, large conotoxin, tryptase inhibitor, and anti-microbial peptide.

In certain embodiments, all or substantially all of the DRPs within a DRP scaffold library have an average native overlap of at least 0.5, at least 0.6, at least 0.7 or at least 0.8 with a consensus or centroid DRP amino acid sequence for that DRP scaffold library. In certain embodiments, the consensus or centroid DRP amino acid sequence for each DRP scaffold library is an amino acid sequence shown in FIG. 8 or FIG. 9, wherein X indicates any amino acid. In particular embodiments, the DRPs within each DRP scaffold library comprise a sequence having at least 80%, at least 90%, at least 95%, or 100% identity to a sequence shown in FIG. 8 or FIG. 9, wherein X indicates any amino acid.

In certain embodiments, all or substantially all of the DRPs within each of the DRP scaffold libraries have an average native overlap of less than 0.5, less than 0.4, or less than 0.3 with the consensus or centroid DRP amino acid sequence of other DRP scaffold libraries.

In certain embodiments, the present invention includes methods of producing one or more DRP scaffold libraries. In particular embodiments, DRP scaffold libraries are produced by mutagenizing one or more codons within a polynucleotide encoding a core or consensus DRP within one of the 20 DRP clusters identified herein, e.g., by introducing one or more modifications to the polynucleotide sequence, such as one or more nucleotide insertions or substitutions. In particular embodiments, the mutagenesis results in the introduction of one or more amino acid modifications as compared to the core consensus DRP. In particular embodiments, a library of polynucleotides encoding DRPs is generated, with various encoded DRPs having different amino acid modifications. In particular embodiments, the libraries are generated using random mutagenesis techniques. In certain embodiments, the nucleic acid modifications are made to codons encoding the DRP. In particular embodiments, the modifications are made to codons for amino acids located within specified regions of the DRPs, e.g., to maintain the DRP scaffold structure, such as to maintain one or more shared structural features of the DRPs within the cluster from which the library was generated. For instance, nucleic acid modification may be made to codons for amino acids not involved in disulfide bonds. In particular embodiments, modifications are made to codons for amino acids located in a region of a DRP predicted to be available for binding to a target of interest, e.g., a contact surface or an outer surface of a DRP. In certain embodiments, the modified codons encode regions of the DRP that are not sequence-conserved. In particular embodiments, the modifications are made to amino acid residues that are not highly conserved between DRPs within a scaffold. In particular embodiments, the dark regions of each DRP cluster shown in FIG. 4 indicate illustrative surfaces to modify. These non-conserved regions are often on loops and surface exposed areas, which make them more likely to be flexible and suggests that modifying them will not affect the fold of the peptide, which is driven largely by disulfide bonding but could be disrupted by modifications in conserved core residues. In certain embodiments, each DRP or DRP scaffold will only have a single surface matching these conditions due to their small size, which results in a limited number of residues from which to choose for variation.

In particular embodiments, the representative DRP within each DRP cluster is mutagenized at the amino acid residues of contact surfaces in the representative or consensus DRP scaffold sequences shown in FIG. 8 or FIG. 9.

In certain embodiments, the present invention, the DRP libraries comprise fusion proteins comprising a DRP described herein and a second polypeptide sequence. In certain embodiments, the second polypeptide sequence is a polypeptide, or fragment thereof, that localizes to the surface of a cell or microorganism, such as a yeast or bacterial cell surface or cell coat protein, a phage surface or coat protein, or a viral capsid protein, including but not limited to any of those described herein. In particular embodiments, the fusion protein localizes to the surface of the microorganism, e.g., yeast or phage, in a manner that displays the DRP on the surface that permits it to bind to cognate binding partners, including targets of interest.

Yeast offer multiple options for cell surface anchor proteins, including Agα1p, Aga2p, Cwp1p, Cwp2p, Tip1p, Flo1p, Sed1p, YCR89w, and Tir1. A DRP may be fused to the C- or N-terminus of an anchor protein. The choice of the anchor protein and fusion terminus depends on the protein to be engineered; generally the terminus farthest from the functional portion of the protein should be tethered to the anchor protein to avoid disrupting activity. The most common yeast display system employs fusion of the protein of interest to the C-terminus of the α-agglutinin mating protein Aga2p subunit, a technology pioneered by Boder and Wittrup. Yeast surface display constructs may include one or more epitope tags: e.g., a hemagglutinin (HA) tag between Aga2p and the N-terminus of the DRP of interest, and a C-terminal c-myc tag. Induction of protein expression results in surface display of the fusion protein through disulfide bond formation of Aga2p to the β1,6-glucan-anchored Aga1p domain of α-agglutinin. The epitope tags allow quantification of fusion protein expression, and thus normalization of protein function to expression level by flow cytometry using fluorescently labeled antibodies.

Examples of bacteriophage (or simply phage) used in phage display technology include single-stranded DNA viruses that infect a number of gram-negative bacteria. The filamentous phage particles mostly used for display purposes are known as Ff and include strains M13, fl, Fd and ft. Fd phage particle viral mass is approximately 16.3 MDa, and consists mainly of about 2700 copies of the pVIII, a 50 as residue protein encoded by gene VIII. On one side of the phage particle there are 3 to 5 copies of the proteins pVII and pXIX (genes VII and XIX) and on the other side there are 3 to 5 copies of the proteins pIII and pVI. In certain display applications, pIII, a 406 as adsorption protein, is the anchor protein used for peptide expression. The pIII protein appears to have two functional domains: an exposed N-terminal domain that binds the F pilus, but is not required for phage particle assembly, and a C-terminal domain that is buried in the particle and is an integral part of the capsid structure. The C-terminal portion of pVIII is inside the phage particle, close to the DNA, while the N-terminal part is exposed to the surroundings. Most of the currently used phage display vectors use the N-terminus of pIII protein or pVIII protein to display the foreign peptide or protein. The pIII libraries display 3-5 copies of each individual peptide (Scott and Smith 1990), whereas pVIII libraries can display up to 2700 copies of small (up to six amino acids) peptides. The pIII and pVIII proteins can display DRPs of various lengths and cysteine residues can be introduced to the fusion peptide to create conformational constraints by the formation of “loops” between disulfide bridged cysteine residues. Furthermore, the exogenous peptides are well exposed, facilitating the insert-target interactions. Large peptide inserts of up to 38 amino acids can be introduced into the amino terminus of pIII protein without the loss of phage infectivity or particle assembly.

In additional embodiments, the present invention includes polynucleotides that encode the DRPs or fusion proteins described herein, as well as libraries of polynucleotides that encode the DRPs within the DRP scaffold libraries described herein. In particular embodiments, the polynucleotides encode a fusion polypeptide comprising a DRP and an anchor protein or cell surface display protein. The present invention further comprises polynucleotides having one or more nucleotide modifications as compared to these polynucleotides. Polynucleotides of the present invention include polynucleotides that have at least 50%, at least 60%, at least 70%, at least 80%, at least 90%, at least 95%, at least 98%, or at least 99% sequence identity to a polynucleotide that encodes a DRP or fusion protein described herein, including those comprising DRP variants comprising one or more amino acid modifications as compared to a native or core DRP. In certain embodiments, the polynucleotides are codon-optimized, e.g., to enhance expression of the encoded DRP in a microorganisms, e.g., a yeast cell, bacterial cell, or phage. Methods of codon-optimization are known in the art.

In particular embodiments, the present invention includes vectors comprising a polynucleotide that encodes a DRP or fusion protein described herein, as well as libraries of vectors comprising polynucleotides that encode the DRPs within the DRP scaffold libraries described herein. In particular embodiments, the vectors are expression vectors that further comprise one or more regulatory element, e.g., a promoter, that direct expression of the DRP or fusion protein in a microorganism, e.g., a bacterial cell, a yeast cell, or a phage. In certain embodiments, a DRP library comprises polynucleotides encoding DRPS or DRP fusion proteins from the same cluster or scaffold, and/or variants thereof.

Although most phage display methods have used filamentous phage, lambdoid phage display systems (WO 95/34683; U.S. Pat. No. 5,627,024), T4 phage display systems (Ren, Z-J. et al. (1998) Gene 215:439; Zhu, Z. (1997) CAN 33:534; Jiang, J. et al. (1997) can 128:44380; Ren, Z-J. et al. (1997) CAN 127:215644; Ren, Z-J. (1996) Protein Sci. 5:1833: Efunov, V. P. et al. (1995) Virus Genes 10:173) and T7 phage display systems (Smith, G. P. and Scott, J. K. (1993) Methods in Enzymology. 217, 228-257: U.S. Pat. No. 5,766,905) are also known. Methods of generating peptide libraries and screening these libraries are also disclosed in U.S. Pat. Nos. 5,723,286; 5,432,018; 5,580,717; 5,427,908; and 5,498,530. See also U.S. Pat. Nos. 5,770,434; 5,734,018; 5,698,426; 5,763,192; and 5,723,323.

In one application, bacteriophage (phage), such as filamentous phage, are used to create phage display libraries by transforming host cells with phage vector DNA encoding a library of DRP variants, e.g., a DRP scaffold library. The most common bacteriophages used in phage display are M13 and fd filamentous phage, though T4, T7 and λ phage have also been used. Phagemid vectors may also be used for phage display. The preparation of phage and phagemid display libraries of peptides is now well known in the art. These methods generally require transforming cells with phage or phagemid vector DNA to propagate the libraries as phage particles having one or more copies of the variant peptides or proteins displayed on the surface of the phage particles. The library DNA is prepared using restriction and ligation enzymes in one of several well-known mutagenesis procedures, for example, cassette mutagenesis or oligonucleotide-mediated mutagenesis.

In addition to the widely used yeast S. cerevisiae, yeast surface display platforms also include strains that can utilize methanol as their sole carbon and energy sources, such as Pichia pastoris and Hansenula polymorpha. Yeast display systems are related vector are known and available in the art.

The present invention also includes systems, kits, collections, and master libraries, each of which includes one, two or more DRP scaffold libraries described herein, or one, two or more libraries of polynucleotides or vectors that encode DRPs scaffold polypeptide libraries described herein. In particular embodiments, the system, kit or master library comprises 1 or more, 2 or more, 3 or more, 4 or more, 5 or more, 6 or more, 7 or more, or more, 9 or more, 10 or more, 11 or more, 12 or more, 13 or more, 14 or more, 15 or more, 16 or more, 17 or more, 18 or more, 19 or more, or 20 DRP scaffold libraries or libraries of polynucleotides or vectors that encode DRP scaffold polypeptides. In particular embodiments, any of the libraries are phage display or yeast display libraries. In related embodiments, the present invention includes kits comprising two or more DRP scaffold libraries, or two or more libraries of polynucleotides or vectors that encode DRPs scaffold libraries described herein, wherein each library is present in a separate container. In particular embodiments, the libraries are lyophilized, dried or freeze-dried.

Screening Methods

The present invention also includes methods of screening DRP scaffold libraries and master libraries of the present invention, e.g., to identify a DRP that binds to a polypeptide or other molecule of interest. In particular embodiments, the polypeptide or other molecule of interest is a polypeptide or other molecule associated with a disease or pathological condition in a mammal, such as e.g., a cancer, an inflammatory disease or disorder, an immune-related disease or disorder, a metabolic disease or disorder, a cardiac disease or disorder, a dermatological disease or disorder, an ischemia or reperfusion injury or disorder, or an injury or disease of soft tissue, cartilage, or bone. While description of screening methods herein uses a target polypeptide for illustrative purposes, it is understood that the methods could be employed to identify DRPs that bind to other types of molecules, such as small organic compounds.

In particular embodiments, methods of screening comprise contacting a polypeptide of interest with one or more DRP scaffold libraries of the present invention, e.g., under conditions and for a time duration sufficient to allow binding of a DRP to the polypeptide of interest, and detecting binding (or an amount of binding) of a DRP to the polypeptide of interest, wherein the presence of binding of the DRP to the polypeptide of interest (e.g., as compared to the absence of binding of a control peptide or other DRPs in the library), or a greater amount of binding of the DRP to the polypeptide of interest (e.g., as compared to the amount of binding of a control DRP or other DRPs in the library) indicate that the DRP binds to the polypeptide of interest. In particular embodiments, the control peptide in a non-DRP, a DRP that does not fall within the DRP scaffold library, or a peptide known to not specifically bind to the target polypeptide of interest. In particular embodiments of the methods of screening disclosed herein, the peptide of interest is contacted with 2 or more, 3 or more, 4 or more, 5 or more, 6 or more, 7 or more, or more, 9 or more, 10 or more, 11 or more, 12 or more, 13 or more, 14 or more, 15 or more, 16 or more, 17 or more, 18 or more, 19 or more, or 20 DRP scaffold libraries described herein. In particular embodiments, screening methods comprise contacting the polypeptide of interest with a master library comprising two or more DRP libraries described herein.

In particular embodiments, the DRP libraries are screened using cell surface display methods, e.g., phage or yeast display methods. Such methods are known in the art. In particular embodiments, the peptide of interest is immobilized on a solid support. In particular embodiments, the polypeptide of interest and/or the DRPs (or fusion proteins thereof) are labeled, e.g., with a fluorescent label. In particular embodiments, the peptide of interest and/or the DRPs (or fusion proteins thereof) are conjugated to a detectable label or comprise a detectable sequence, e.g., a sequence that binds to a labeled moiety.

In certain embodiments, the DRP scaffold library is phagemid based, containing an arabinose promoter driving the expression of fusion proteins of the following form: an STII secretion signal, followed by a hemagglutinin tag, a linker (e.g., a four residue linker sequence), the DRP library, another linker (e.g., a four residue linker sequence), and the M13 gene-3 coat protein. In certain embodiments, the phagemid DRP libraries are amplified using oligonucleotides containing the variable positions encoded by NNK codons. The DNA fragments encoding the desired scaffolds were then cloned into the phagemid vector and transformed into electrocompetent E. coli XL1-Blue cells

In various embodiments, screening methods of the present invention further comprise one or more additional steps, such as isolating the DRP (or fusion protein thereof) that binds to the target of interest, cloning the polynucleotide sequence encoding the binding DRP (or fusion protein thereof), and/or sequencing the binding DRP or polynucleotide sequence encoding the binding DRP (or fusion protein thereof). In particular embodiments, a polynucleotide sequence encoding the binding DRP (or fusion protein thereof) is subcloned into an expression vector, e.g., an in vitro expression vector or an in vivo expression vector. In particular embodiments, the binding DRP (or fusion protein thereof) is expressed in vitro or in vivo (e.g., in bacterial or mammalian cells), purified, and/or assayed for binding to the peptide of interest. In particular embodiments, the binding DRP (or fusion polypeptide) is assayed for its ability to modulate, e.g., antagonize or agonize, one or more biological activities or the target of interest. In certain embodiments, the binding DRP (or fusion polypeptide thereof) is assayed for therapeutic effect on a disease or disorder associated with the target polypeptide of interest, e.g., in vitro, ex vivo, or in vivo (e.g., in an animal model of the disease or disorder or a human patient diagnosed with the disease or disorder). Methods of assaying for binding are well known in the art and include, e.g., ELISA, immunoprecipitation, FACs, mass spectrometry and other assays. Methods of assaying biological activity and therapeutic effect vary depending upon the target of interest but are generally known in the art when a particular target of interest is associated with a particular biological activity, disease or disorder.

In particular embodiments, DRP scaffold libraries are screened by yeast or phage display. In certain embodiments, the DRPs are fused to a phage surface or coat protein, such as, e.g., M13. Cell surface display screening methods, including yeast and phage display technologies, have been extensively characterized previously. For example, for phage display, a DNA library encoding a DRP (e.g., a representative DRP of a cluster described herein), with some of the codons randomized (i.e., mutagenized), is ligated into a phage plasmid in a gene encoding for a coat protein. This process results in a library of phage expressing diversified protein sequences on their surface, e.g., fusion proteins comprising a phage coat protein thereof and a DRP. The library is then panned against an immobilized protein target of interest. Phage particles with proteins that bind the target are selected over those that do not in an iterative panning process. After a few rounds of panning, the selected phage clones are sequenced and the peptides corresponding to those sequences are synthesized and assayed to confirm binding. A number of studies have used DRPs as phage library scaffolds (reviewed in [28]), and the rationale design and development of potent IL-6 antagonist compounds have been identified using this method.

In certain embodiments, screening methods comprise further mutagenizing a DRP identified as binding a desired target upon screening one or more or two or more DRP scaffold libraries, to generate a further DRP scaffold library. This library may then be screened to identify additional DRPs that bind to the target.

In related embodiments, screening methods of the present invention further comprise an additional step of producing one or more DRP scaffold libraries.

### EXAMPLES

### Example 1

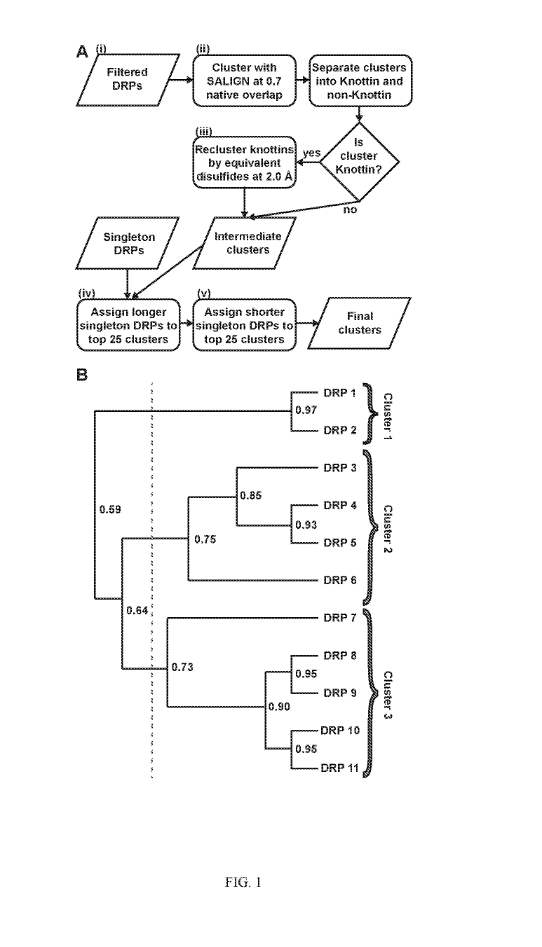

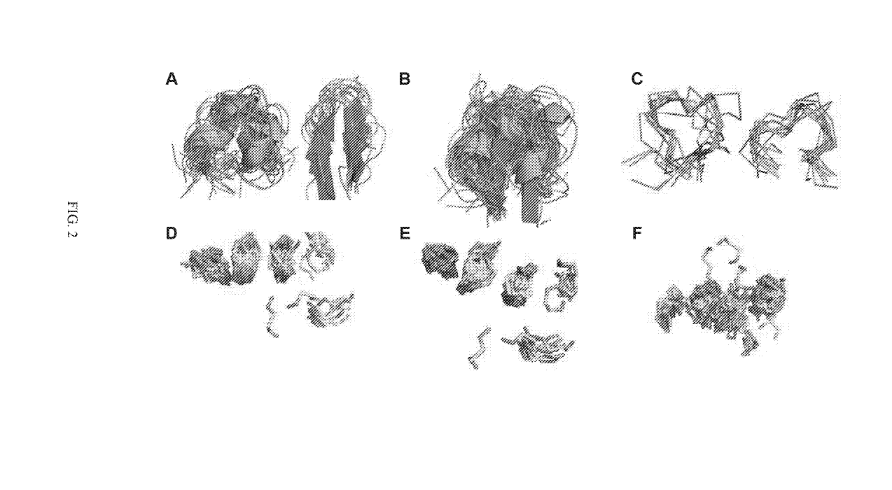

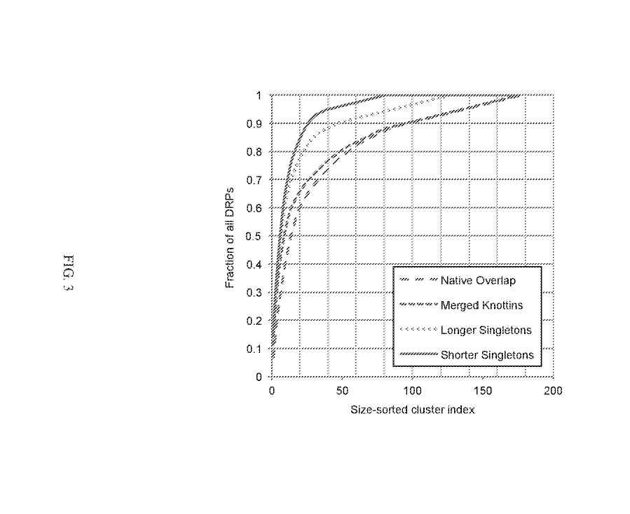

This Example describes the identification of 20 distinct DRP clusters or scaffolds. The method used to analyze known DRPs and assign them to distinct clusters consisted of five steps: filtering, hierarchical clustering using native overlap as the distance metric, reclustering knottins using disulfide distance as distance metric, re-assigning longer singletons, and re-assigning shorter singletons (FIG. 1; additional details in Methods).

Filtering Identical DRPs

The Protein Data Bank (PDB) was searched for individual chains with fewer than 50 residues and between one and four annotated disulfide bonds, with 1,411 DRPs fitting these criteria. This initial dataset was filtered to remove identical DRPs, including 292 identical insulin chains, resulting in 806 representative structures (FIG. 1A; step i).

Native Overlap Clustering and Cutoff Determination

The 806 representative DRPs were clustered using native overlap as the distance metric in an average-linkage hierarchical clustering algorithm (FIG. 1B; Methods). The algorithm terminated when the smallest average native overlap between any two clusters was below a cutoff. The value of this cutoff was determined by trial-and-error, selecting the optimal cutoff of 0.7 through visualization of clusters with 3D structure viewing software (FIG. 2A). A cutoff of 0.6 was considered, but it resulted in assigning DRPs with clearly different folds to the same cluster; for example, small n-hairpins, which have tight turns in between successive β-strands, clustered with conotoxins, which are similar to hairpins in size but have rounded turns connecting loops or helices (FIG. 2B). On the other hand, a cutoff of 0.8 was too stringent, assigning DRPs with very similar structures into different clusters (FIG. 2C). The average-linkage hierarchical clustering step using the selected native overlap cutoff of 0.7 grouped the 806 DRPs into 178 clusters (FIG. 1A, step ii).

Knottin Reclustering and Cutoff Determination

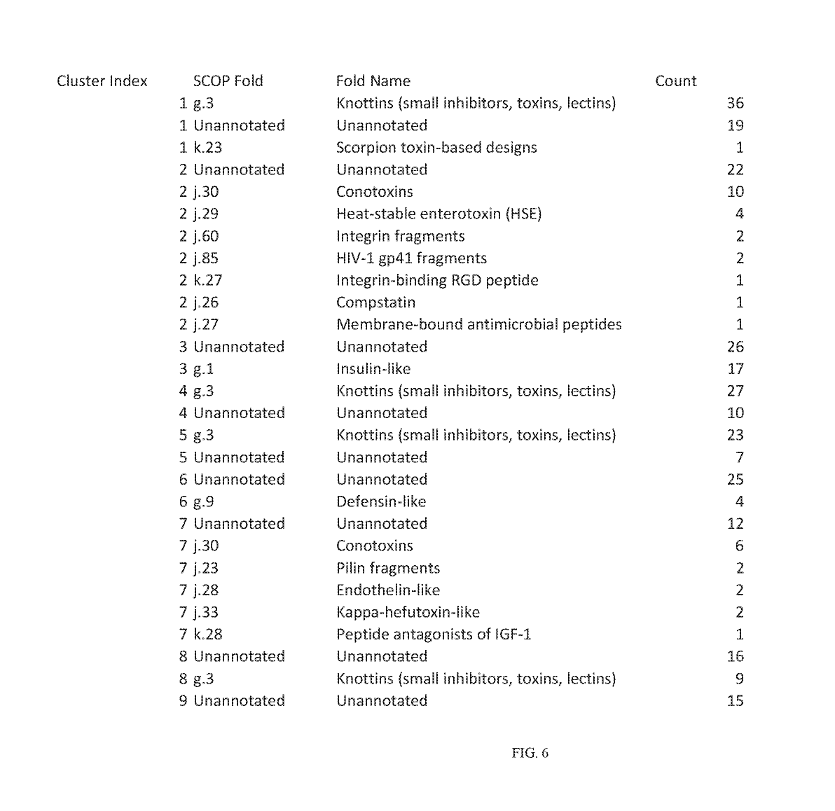

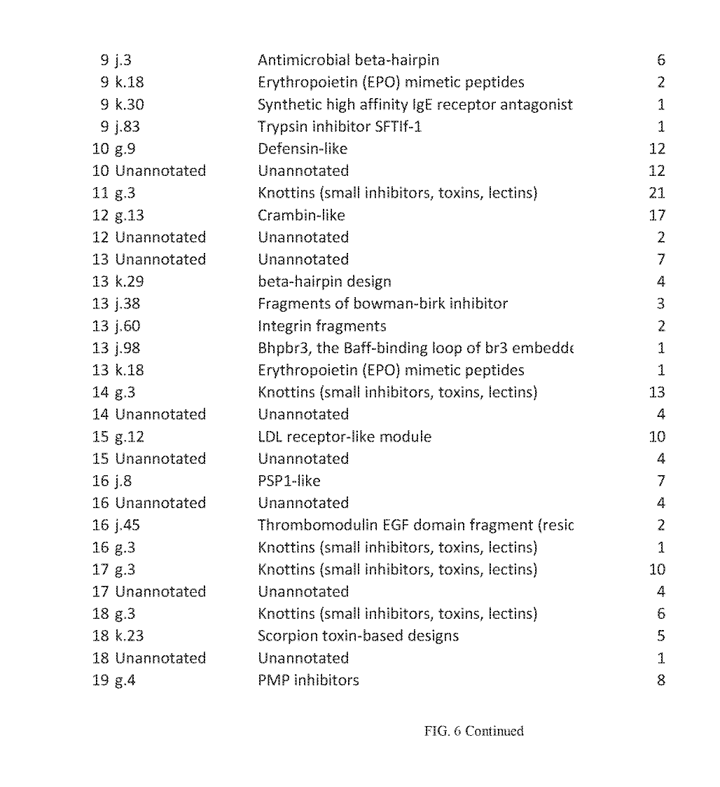

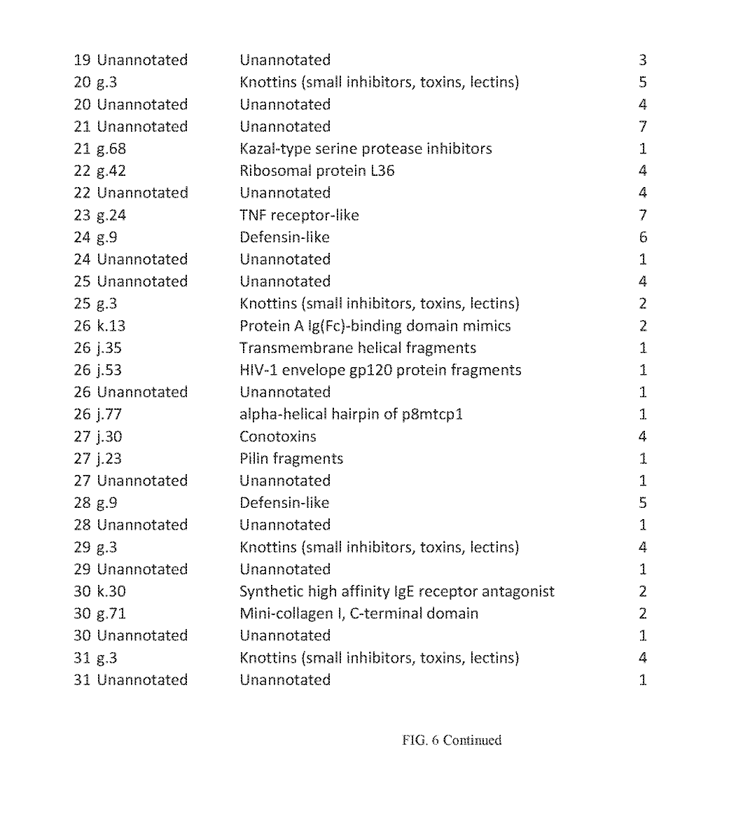

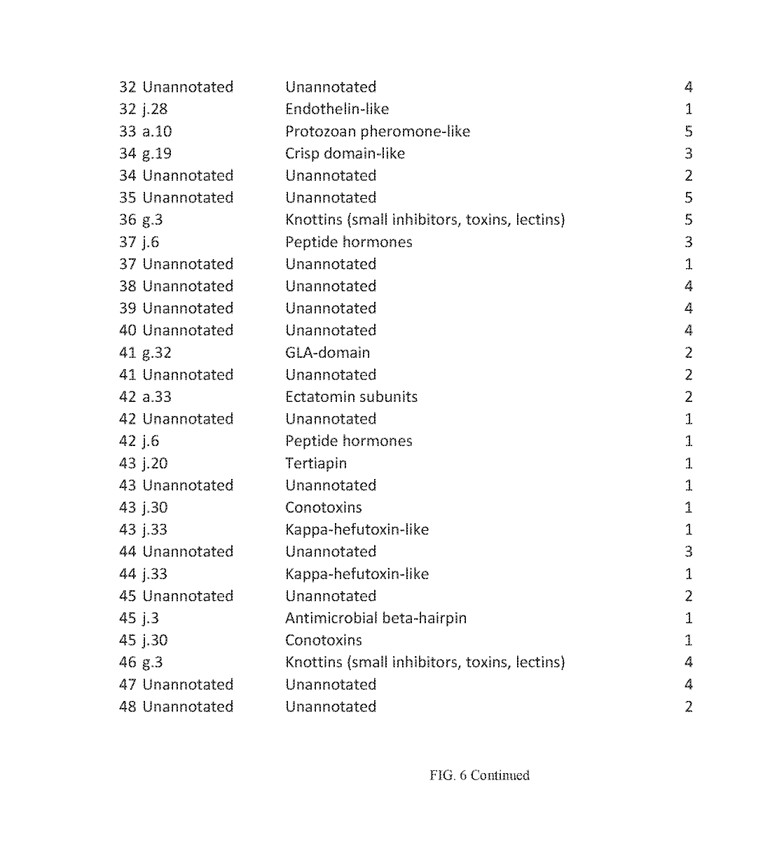

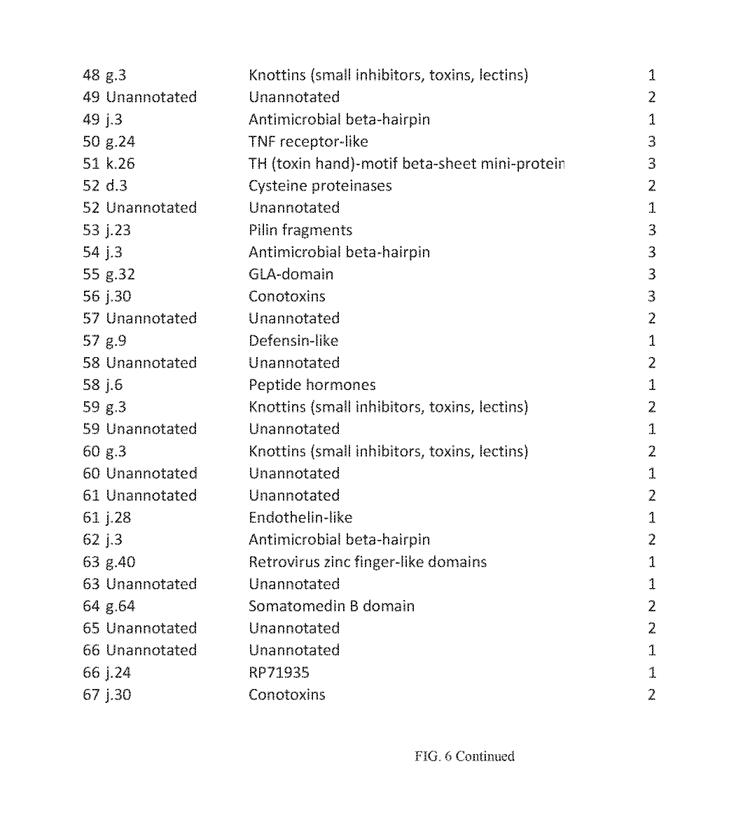

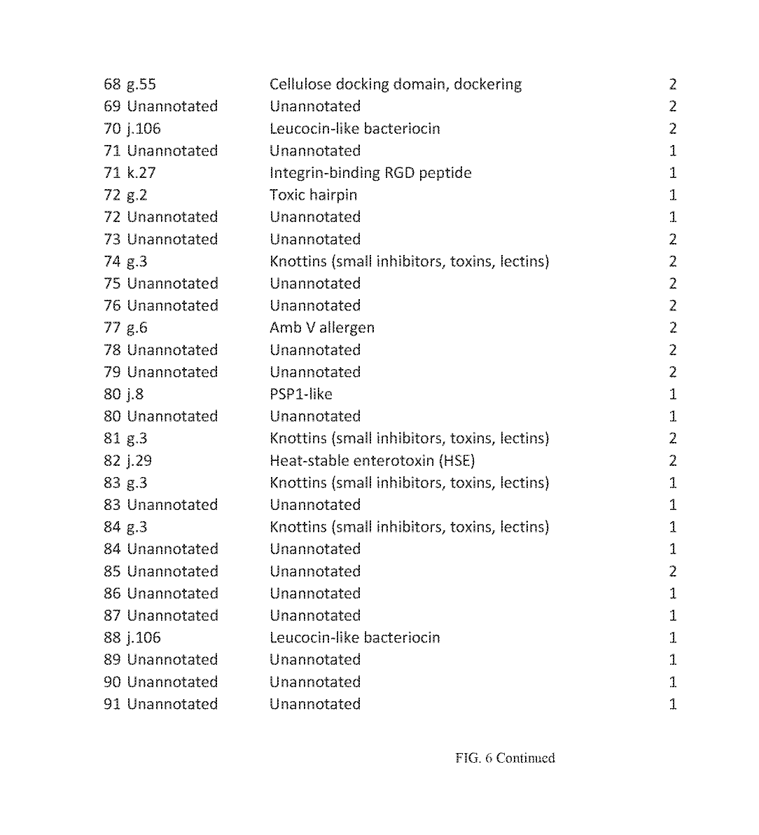

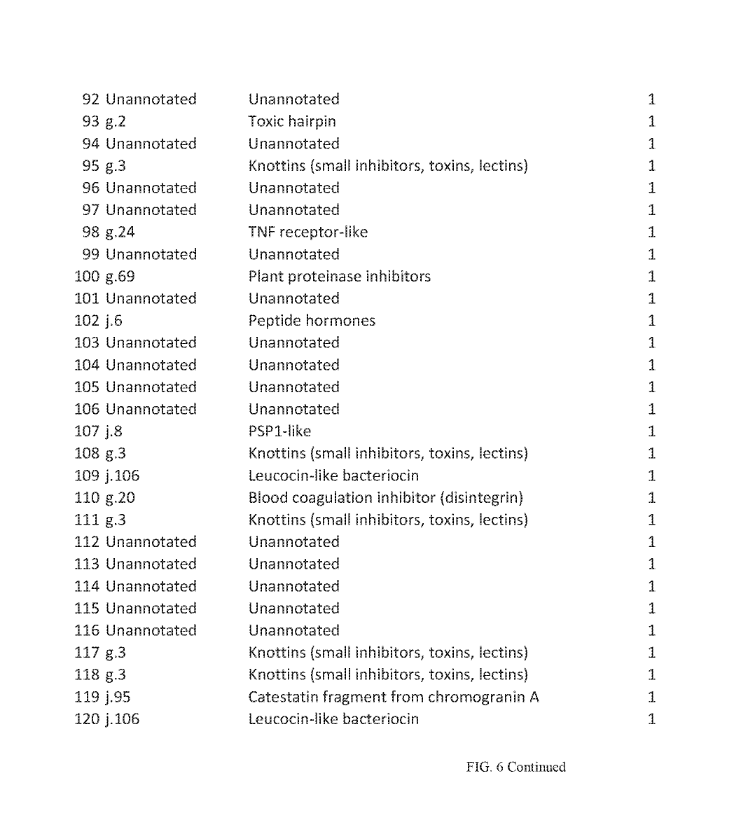

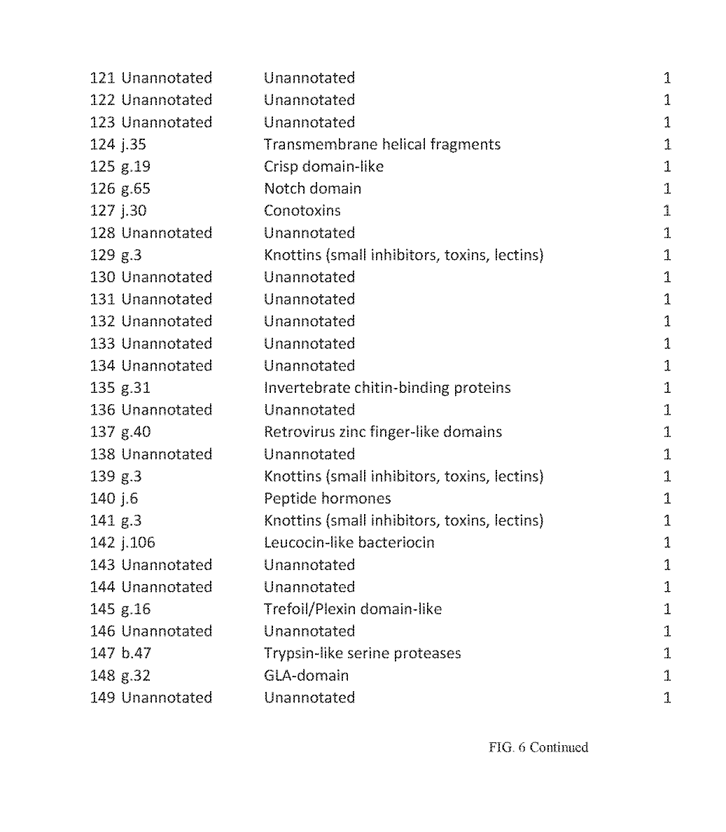

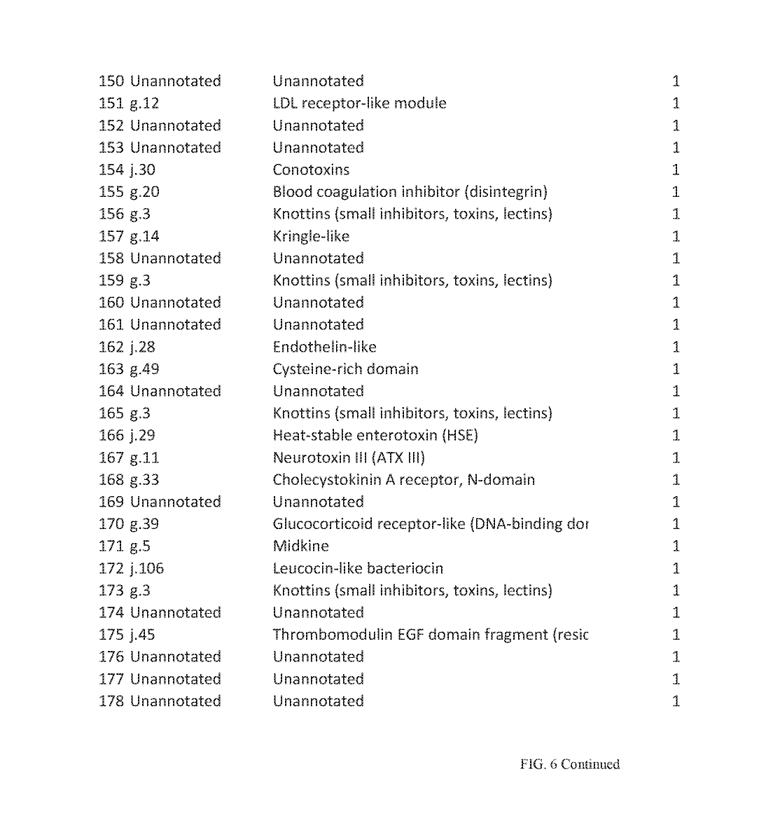

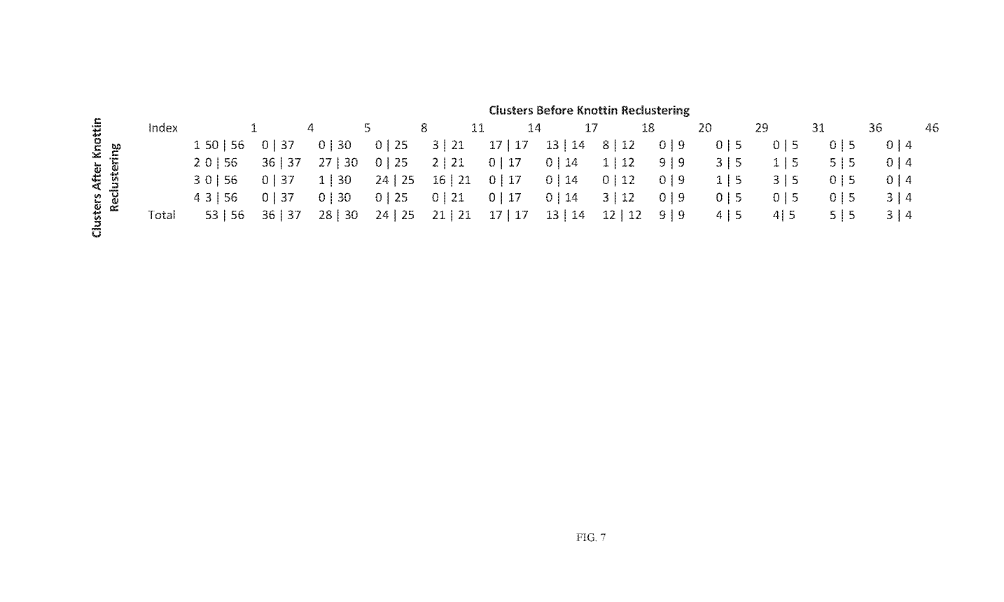

Peptides were annotated with SCOP identifiers [39]. DRPs with the same SCOP fold identifiers were generally in the same clusters, validating the clustering procedure (FIG. 6). However, the 260 ‘knottins’ (SCOP ID g.3) were classified into 15 distinct clusters, due to their varied loop lengths. In phage display experiments, loop lengths may be varied as part of the library creation, and the core structures of the scaffolds are of greater importance. Therefore, knottins were reclustered by their core disulfide bond structure only (FIG. 1A, step iii), as follows.

Clusters containing four or more DRPs annotated with the knottin fold were given as input to the average-linkage hierarchical clustering algorithm, here using the distance between equivalent disulfide bonds as the distance metric (Methods). A disulfide distance cutoff of 2.0 Å was again selected by trial-and-error. This cutoff resulted in high structural overlap of disulfide bonds across DRPs in the knottin clusters (FIG. 2D) with a separation of ˜1.8 Å between consecutive groups of bonds in the most populated cluster despite 91 members being present. The cutoff of 1.5 Å resulted in a similar separation, but here, only 64 members were in the most populated cluster (FIG. 2E), resulting in suboptimal lower coverage. The cutoff of 2.5 Å led to 131 members in the most populated cluster, but there was no clear visual separation apparent in consecutive groups of disulfide bonds (FIG. 2F). This cutoff would likely render selection of a representative scaffold problematic, as there would be no DRP in the cluster that possessed a set of disulfide bonds structurally equivalent to all other members of the cluster. The optimal cutoff of 2.0 Å reduced the number of clusters containing four or more knottins from 15 to 4 (FIG. 7). Together with all non-knottin clusters produced in step ii, there were 176 intermediate DRP clusters (FIG. 3).

Singleton Reassignment

It was observed that some DRPs in less-populated clusters had native overlaps above 0.7 when aligned to peptides in clusters with more members. However, the hierarchical nature of the procedure grouped the most similar DRPs together with each iteration; this process sometimes resulted in a DRP being grouped with its closest neighbor in a small cluster even if there was another more populated cluster containing members similar to that DRP. Provided the DRP aligned to at least one peptide in the larger cluster at a native overlap of 0.7, the DRP was reassigned (i.e., a singleton) to the larger cluster. This post-processing refinement increased the sizes of the most-populated clusters (FIG. 1A, steps iv-v), and reduced the total number of clusters from 176 to 81 (FIG. 3). The full composition of all clusters is available in FIG. 8.

Twenty Structure Folds Represent the Majority of DRPs

A primary goal of the clustering procedure was to identify a small number of representative DRPs, as this goal balanced a number of peptide scaffolds large enough to cover a significant fraction of DRP structure space but small enough to be experimentally tractable in phage display experiments. The method resulted in 84.5% of DRPs in the PDB being assigned to the top 20 most populated clusters (FIG. 3). Although 81 distinct DRP folds were identified, the least populated 61 clusters each contained only nine or fewer DRPs, with 43 of these clusters containing a single peptide. It is feasible to construct 20 phage libraries, which would be structurally representative of nearly 85% of all DRPs whose structures have been solved. Images of these top 20 clusters ranked by membership are presented in FIG. 4.

Cluster Representatives are Structurally Diverse

From each of the top 20 clusters, the DRP with the largest average native overlap to all other members of that cluster was identified; this DRP was selected as the representative member of the cluster and considered a potential scaffold for phage display. One goal of the study was to select representative DRPs that were structurally distinct from the other representatives, which was assessed by the average native overlap between the representatives. All representative DRPs had an average native overlap to other DRPs in their clusters of greater than 0.64; the median value across the 20 clusters was 0.77 (Table 1, diagonal values). In contrast, the median native overlap for pairs of representatives was only 0.39 (Table 1, off-diagonal values), indicating that the clustering procedure indeed resulted in a structurally diverse set of DRP clusters that were truly representative of the majority of known DRP structure space. Additional details of each cluster are presented in Table 2.

TABLE 1
Matrix of structure diversity across clusters. For each cluster, the DRP with the
highest average native overlap value to all other DRPs in the cluster (the “centroid”) was
selected as the representative member to be used as the basis for phage display libraries (the
calculation of native overlap is described in Methods). These average native overlap values
for the representative DRPs are displayed along the matrix diagonal in bold. Additionally,
pairwise structural alignments of all representatives were computed with SALIGN; the
resulting native overlap values are displayed in off-diagonal cells in the matrix.

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| 1 | .73 | .64 | .42 | .25 | .67 | .39 | .50 | .31 | .67 | .42 | .31 | .41 | .53 | .34 | .69 | .39 | .39 | .33 | .39 | .58 |
| 2 | .64 | .76 | .39 | .27 | .73 | .42 | .58 | .39 | .48 | .47 | .33 | .24 | .42 | .47 | .70 | .70 | .50 | .27 | .45 | .70 |
| 3 | .42 | .39 | .85 | .52 | .34 | .38 | .48 | .67 | .40 | .36 | .38 | .41 | .50 | .32 | .46 | .33 | .39 | .57 | .32 | .33 |
| 4 | .25 | .27 | .52 | .77 | .31 | .53 | .47 | .75 | .33 | .31 | .67 | .26 | .31 | .26 | .32 | .24 | .31 | .63 | .25 | .33 |
| 5 | .67 | .73 | .34 | .31 | .79 | .38 | .55 | .38 | .37 | .42 | .38 | .37 | .34 | .45 | .69 | .61 | .44 | .45 | .23 | .77 |
| 6 | .39 | .42 | .38 | .53 | .38 | .71 | .74 | .53 | .50 | .33 | .65 | .22 | .28 | .34 | .54 | .45 | .36 | .47 | .34 | .37 |
| 7 | .50 | .58 | .48 | .47 | .55 | .74 | .66 | .53 | .33 | .39 | .58 | .26 | .34 | .45 | .61 | .58 | .50 | .47 | .27 | .57 |
| 8 | .31 | .39 | .67 | .75 | .38 | .53 | .53 | .80 | .40 | .31 | .50 | .30 | .41 | .37 | .39 | .27 | .42 | .63 | .25 | .37 |
| 9 | .67 | .48 | .40 | .33 | .37 | .50 | .33 | .40 | .91 | .72 | .30 | .33 | .38 | .39 | .50 | .55 | .39 | .37 | .43 | .63 |
| 10 | .42 | .47 | .36 | .31 | .42 | .33 | .39 | .31 | .72 | .91 | .25 | .50 | .36 | .42 | .56 | .42 | .47 | .33 | .34 | .28 |
| 11 | .31 | .33 | .38 | .67 | .38 | .65 | .58 | .50 | .30 | .25 | .80 | .20 | .19 | .29 | .39 | .33 | .31 | .47 | .25 | .33 |
| 12 | .41 | .24 | .41 | .26 | .37 | .22 | .26 | .30 | .33 | .50 | .20 | .98 | .41 | .35 | .30 | .30 | .28 | .22 | .30 | .30 |
| 13 | .53 | .42 | .50 | .31 | .34 | .28 | .34 | .41 | .38 | .36 | .19 | .41 | .64 | .29 | .53 | .45 | .25 | .31 | .36 | .38 |
| 14 | .34 | .47 | .32 | .26 | .45 | .34 | .45 | .37 | .39 | .42 | .29 | .35 | .29 | .77 | .42 | .45 | .50 | .32 | .48 | .55 |
| 15 | .69 | .70 | .46 | .32 | .69 | .54 | .61 | .39 | .50 | .56 | .39 | .30 | .53 | .42 | .73 | .64 | .44 | .36 | .32 | .60 |
| 16 | .39 | .70 | .33 | .24 | .61 | .45 | .58 | .27 | .55 | .42 | .33 | .30 | .45 | .45 | .64 | .90 | .42 | .30 | .27 | .61 |
| 17 | .39 | .50 | .39 | .31 | .44 | .36 | .50 | .42 | .39 | .47 | .31 | .28 | .25 | .50 | .44 | .42 | .75 | .39 | .43 | .47 |
| 18 | .33 | .27 | .57 | .63 | .45 | .47 | .47 | .63 | .37 | .33 | .47 | .22 | .31 | .32 | .36 | .30 | .39 | .79 | .30 | .47 |
| 19 | .39 | .45 | .32 | .25 | .23 | .34 | .27 | .25 | .43 | .34 | .25 | .30 | .36 | .48 | .32 | .27 | .43 | .30 | .77 | .36 |
| 20 | .58 | .70 | .33 | .33 | .77 | .37 | .57 | .37 | .63 | .28 | .33 | .30 | .38 | .55 | .60 | .61 | .47 | .47 | .36 | .79 |

Sequence Dissimilarity of Peptides in the Same Cluster

To examine the relationship between structure and sequence in the clusters, the average pairwise sequence identity among all members in each cluster was calculated (Table 2). No cluster in the top 20 had average sequence identity higher than 60%, and ten of the clusters had average sequence identity of less than 25%, indicating that the binding partners of DRPs within the same cluster were likely diverse. Sequence conservation was visualized by coloring the DRP structures according to the degree of conservation at each residue position (FIG. 4). In most of the clusters, the light gray conserved areas consisted of residues surrounding the disulfide bonds which likely contribute to DRP folding and stability, while the wide, medium gray diverse areas were generally found in the loops and surface regions, which are more likely to interact with other proteins and are viable candidate regions for randomization through phage display.

TABLE 2
Summary of clusters.

| | | | Avg | Avg | |
|---|---|---|---|---|---|
| Cluster | Name | # Members | Seq Id | Length | Scaffold |
| 1 | Knottin I | 115 | 21.8 | 38 | 2crdA |
| 2 | Knottin II | 98 | 23.4 | 24 | 2jtbA |
| 3 | Insulin | 58 | 42.4 | 23 | 3gkyC |
| 4 | Small Conotoxin | 52 | 24.9 | 12 | 1e76A |
| 5 | Knottin III | 48 | 30.9 | 30 | 3e4hA |
| 6 | Small Hairpin | 42 | 15.9 | 16 | 1wo0A |
| 7 | EGF-like Hairpins | 39 | 17 | 19 | 2oqjL |
| 8 | Medium Conotoxin | 35 | 21 | 17 | 2uz6K |
| 9 | α-Defensin | 30 | 53.4 | 31 | 3lo2B |
| 10 | β-Defensin | 25 | 51.1 | 37 | 2nlsA |
| 11 | Large Hairpin | 22 | 21.5 | 17 | 1gm2A |
| 12 | Crambin | 19 | 56.8 | 46 | 1orlA |
| 13 | Helix-Loop-Helix | 19 | 12.2 | 34 | 1bzbA |
| 14 | LDL Receptor | 17 | 30.4 | 39 | 2kriB |
| 15 | Knottin IV | 12 | 19.7 | 29 | 1du9A |
| 16 | PMP Inhibitors | 11 | 59 | 35 | 2f91B |
| 17 | TNF Receptor | 11 | 42.6 | 39 | 1xu2R |
| 18 | Large Conotoxin | 10 | 24.6 | 19 | 1tckA |
| 19 | Tryptase Inhibitor | 10 | 42.2 | 39 | 2kmoA |
| 20 | Anti-microbial | 9 | 49.2 | 30 | 1mmcA |
| | peptide | | | | |

Clusters are sorted by number of members.
Name: Manually assigned name, derived from the most frequent
SCOP fold assignment for each cluster.
Avg Seq Id: Average pairwise sequence identity of all DRPs in the cluster.
Avg Length: Average sequence length of all DRPs in the cluster, derived from the sequence resolved in the PDB structure.
Scaffold: Selected representative for the cluster.

Panning Against Human Interleukin-23

To demonstrate the utility of the clustering approach described herein, phage display libraries were constructed for three representative DRPs and panned against the human cytokine protein interleukin-23 (IL-23). Inhibiting IL-23 binding to its receptor (IL-23R) reduces inflammation and other adverse immune responses; thus, IL-23 is an attractive therapeutic target [40]. The interaction between IL-23 and IL-23R is a typical protein-protein interaction involving a large flat recognition surface, and a low molecular weight binder to IL-23, which would prevent complex formation, would be challenging to discover.

The three selected DRPs were (1) from the large conotoxin cluster, an antagonist of vascular endothelial growth factor (PDB identifier 1KAT[41]); (2) from the small hairpin cluster, an agonist of erythropoietin (1KVF[42]), and (3) from the helix-loop-helix cluster, a derivative of Protein-A (1ZDC [43]). These DRPs were chosen due to their different secondary structure classes (loops, sheet, helix respectively), which may maximize their binding diversity. Moreover, the first two peptides were themselves products of phage display libraries, which suggested experimental tractability. One library was designed for each DRP cluster; these libraries were referred to by the PDB identifiers of their clusters or scaffolds (IKATr1, 1KVFr1, and 1ZDCr1; full library design described in FIGS. 5A-C and FIG. 9).

The libraries were constructed and screened against immobilized IL-23 in successive rounds of panning. Enrichment ratios, which compare the output titers in the target selection to a background negative control, were determined after each round. Panning the IKATr1 and 1KVFr1 libraries was halted after four rounds due to the lack of enrichment; however, the 1ZDCr1 library showed significant enrichment after the fourth round and was thus subjected to two further rounds of panning (FIG. 5D). After the sixth round, individual clones were isolated and assessed for binding with a phage ELISA. Positive clones were sequenced. One sequence, Peptide 1, was synthesized and tested in a competition ELISA to assess inhibition of IL-23 binding to IL-23R. Peptide 1 is a 34 amino acid peptide comprising Cys at amino acid positions 5 and 34 (counting from the N-terminus). Peptide 1 inhibited binding with an IC₅₀ of 3.3 μM (FIG. 5E); it is likely that this binding potency could be improved through medicinal chemistry approaches, as has been done previously [34].

The studies described herein established an automated method for clustering all DRPs with solved structure. Most clusters contained DRPs with diversity in sequence, host, and target binders, indicating that cluster members are functionally diverse despite having similar structure. The method resulted in a set of structurally distinct peptide scaffolds, and through careful selection of the surface to vary in phage display, the resulting libraries presented sequence diversity in conformationally distinct, biologically relevant combinations of secondary structure. Thus, these 20 libraries represent the structural diversity of nearly 85% of all DRPs and have the potential to efficiently identify peptide binders to a protein target in a drug discovery program.

Discussion

Through manipulating approximately 4-18 amino acids in a topologically controlled, biologically relevant and defined structure space, in combination with sampling, a large variety of diverse chemistries at each scaffold position, disulfide rich peptides provide a unique solution for the discovery of agonists and antagonists of protein-protein interactions. Phage display's utility for developing hits and ultimately drugs is well appreciated, and its proficiency is derived from the ability to make vast libraries of approximately 10¹⁰ or more sequences and the linkage of genotype to binding phenotype [37].

Advantages of Using Structurally Diverse Scaffolds in Phage Display

The utility of phage display for developing lead compounds is well appreciated, with its proficiency deriving from the ability to make and screen libraries of up to 10¹² sequences and the linkage of genotype to binding phenotype [44]. Typical peptide phage display involves the creation of large libraries sampling enormous sequentially continuous sequence space on unstructured peptides that assume structure only upon binding the target bait. The disordered nature of these peptides weakens the utility of phage display, as in some instances it is impossible to select the weakly active unstructured peptides from the vast majority of inactive peptides.

On the other hand, phage display of DRPs allows for sampling different sequences on a discontinuous surface in conformationally controlled structure space. One of the key requirements in discovering leads, and ultimately drugs, is to present the required functional groups in a sufficient orientation to yield potent and selective molecules at the target of interest, while optimizing the desired drug-like physicochemical features. This requirement is achieved through the common discontinuous surface patches of DRPs, described here, which represent naturally occurring fractions of chemical structure space explored by nature, and as such are biologically relevant. Consequently, the probability of obtaining hits may be higher than with unstructured peptide phage libraries, or with small molecule scaffold topologies explored in combinatorial chemistry, which are typically not biologically relevant. This probability further increases when multiple structurally distinct libraries are panned. To develop such libraries, we require a set of diverse DRP scaffolds, e.g., scaffolds based on the DRP clusters described herein that are used to generate DRP scaffold libraries.

These scaffolds were identified by the protocol in this study, which clusters DRPs by structural similarity over their full length and refines some of the clusters by incorporating the structural conservation of disulfide bonds and by resolving artifacts of the hierarchical clustering method through reassigning singletons to more populated clusters. The result was an experimentally tractable set of 20 structurally diverse, representative scaffolds from the most populated clusters that could be used for constructing phage display libraries.

DRPs are an emerging source of lead compounds in drug discovery due to their inherent chemical and biological stability characteristics, as exemplified by the marketed orally delivered drug linaclotide [29]. DRP phage display libraries may provide a valuable, generic resource for the discovery of additional DRP modulators of protein-protein interactions and may help alleviate the low hit rate currently plaguing the pharmaceutical sector.

Evolutionary Insights Obtained Through DRP Clustering

Clustering DRPs by structure, and incorporating no annotation other than previous identification of some DRPs as knottins, revealed insights into DRP structure. Many clusters contained DRPs possessing structurally conserved disulfide bonds, as demonstrated through qualitative visual analysis. This conservation tends to occur in clusters with larger folds; for example, the EGF-like hairpin, α-defensin, crambin, and TNF receptor clusters all had near-complete conservation of disulfide bonds (FIG. 4). Unlike the knottins, these clusters were aligned over the full length of their sequences in the initial hierarchical clustering step (FIG. 1A, step ii), suggesting that the cysteine bond pairings are indeed the primary contributors to the fold formation. This result corroborated the findings of a previous study that showed high disulfide bond conservation within the SCOP “Small Peptide” fold class [45].

The clusters with the least amount of disulfide bond conservation were those containing peptides with shorter sequence lengths; examples include Small Hairpin and Small Conotoxin clusters. The N and C termini are proximal to each other in both of these folds, and in these peptides there were a number of possible position pairs between which disulfide bonding was sufficient to maintain the structure. Thus, there was less evolutionary pressure to conserve a disulfide bond between specific positions than there is in longer DRPs.

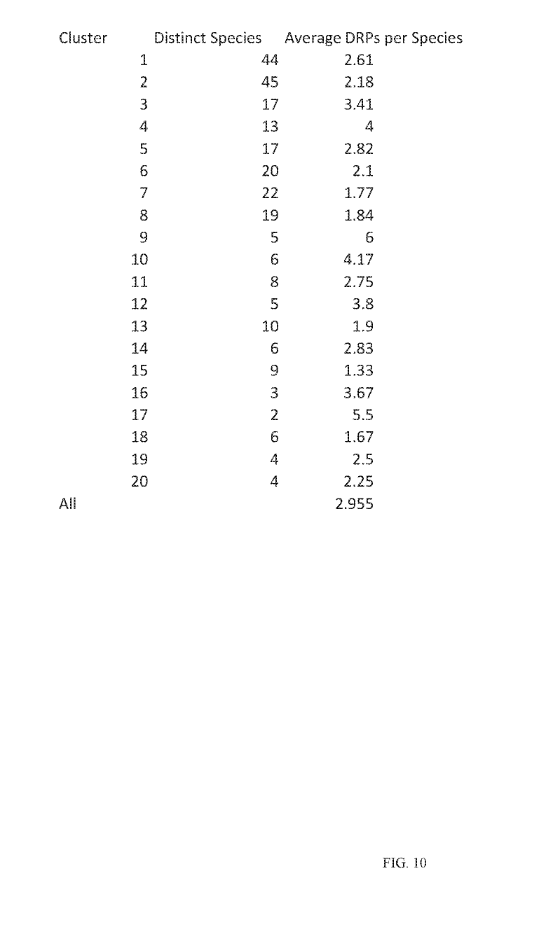

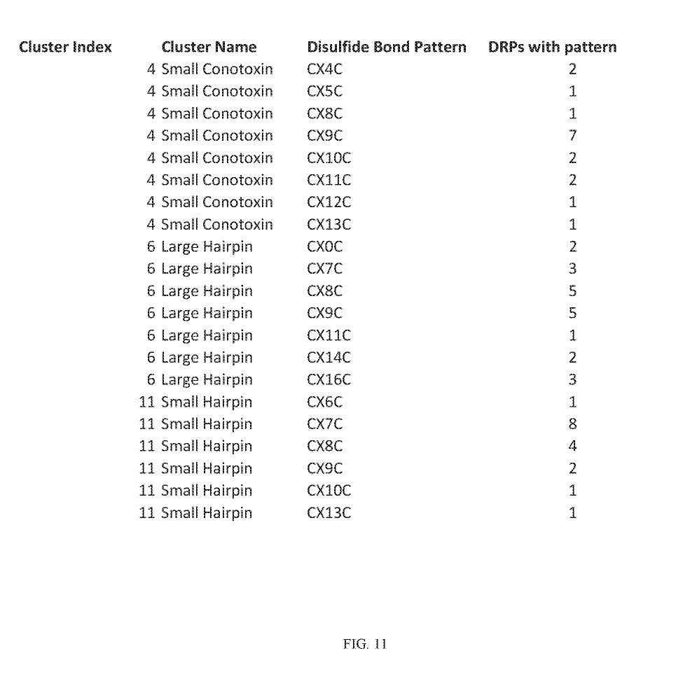

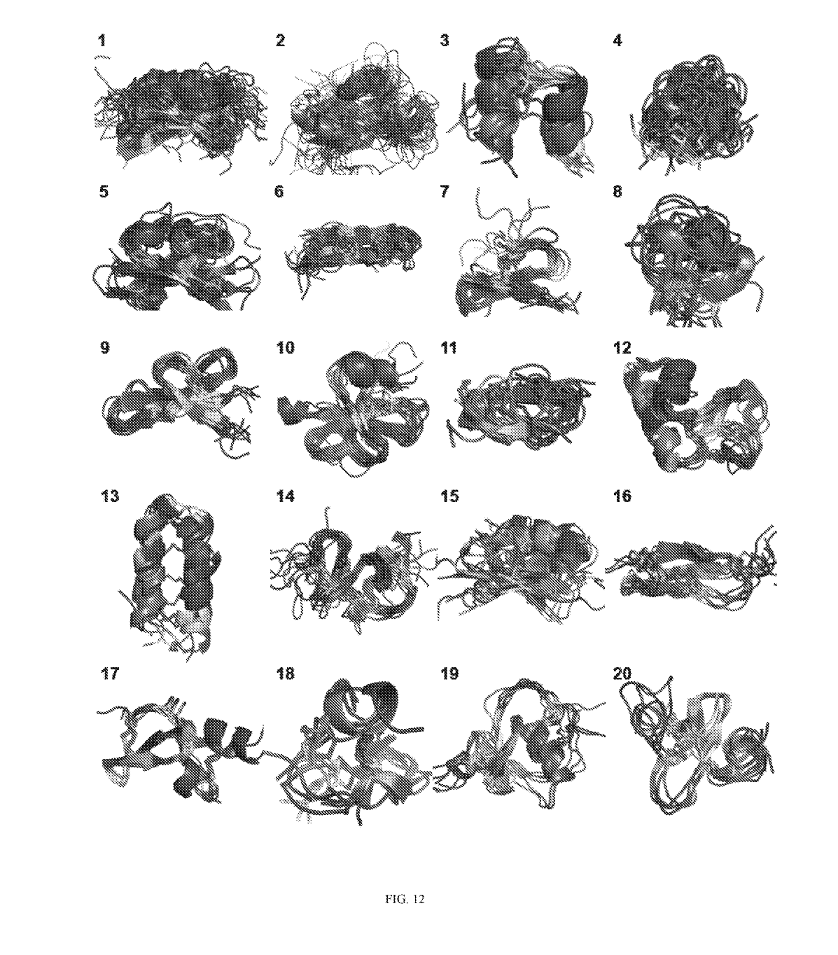

In addition to their utility as phage display libraries, and insight into disulfide bond conservation, the clusters allow for a broader view of DRP evolution. For example, we wondered how DRPs from different species were distributed across the clusters. In a simple analysis, each PDB structure was mapped to its Uniprot accession [46] to obtain its annotated species, and the total number of species, as well as the ratio of DRPs to unique species in each cluster, was calculated (FIG. 10). Most clusters were composed of DRPs expressed across a number of different species. For example, the EGF-hairpin cluster contained 39 peptides from 20 species; the average ratio of DRPs per species across the top 20 clusters was 2.95. This result demonstrates the broad phylogenetic distribution of a small number of DRP folds.

Discussion of Selected Cluster Folds

Detailed analysis of certain clusters may elucidate structure-function relationships among these unique peptides. First, the top 20 clusters included 4 composed primarily of knottin folds. Knottins are characterized by a cysteine-knot architecture; generally, these peptides are composed of an N-terminal helix and two or three C-terminal β-strands, with three disulfide bonds connecting these secondary structure elements [10]. Loops in knottins had high structural variability, rendering these peptides problematic when clustering them by native overlap over the full sequence. Thus, an intermediate step in the protocol reclustered knottins based on structural overlap across their core disulfide bonds, which allowed for selection of a scaffold that was similar in core structure to other members of its cluster, but had the potential to present a binding surface in a similar conformation to a large number of other knottins, particularly if the loop size were to be varied as part of the phage display experiment. Knottin disulfide bonds exhibited a remarkable degree of structural overlap, with 229 DRPs grouped into only 4 clusters (FIG. 7).

Knottins in different clusters generally had different functions. The Knottin I cluster was the largest of all DRP clusters, with 115 members. Of these peptides, 49 were potassium channel inhibitors, drawn from 15 species; 17 were defensins; and 12 assumed an EGF-like fold, 9 of which were found in human coagulation factors (FIG. 8). None of these functions was assigned to DRPs in the Knottin II or III clusters (although five more potassium channel inhibitors were present in Knottin IV). Instead, Knottin II was composed of a diverse array of toxins, including conotoxins, agatoxins, and theraphotoxins, while Knottin III included trypsin inhibitors and cyclotides with antimicrobial functions, predominantly from plants. Thus, core disulfide bond equivalency appeared to correlate strongly with different functions mediated by surface loops across different knottin folds.

Similar to knottins, hairpin peptides fell into multiple clusters: Small Hairpin (averaging 14.3 residues in length) and Large Hairpin (averaging 21.6 residues). Despite these peptides all consisting of simple β-strand pairs joined by one or two disulfide bonds, multiple clusters were created due to the significant differences in sequence lengths, similar to knottins. The Large Hairpin cluster afforded more space along the sequence to incorporate disulfide bonds; peptides in this cluster averaged 1.59 disulfide bonds, compared with 1.22 in the Small Hairpin cluster. Additionally, Large Hairpins were more likely to be found in nature; 70% of cluster members were fully expressed peptides or isolated as a fragment from a full protein. Nearly all of these peptides were serine protease inhibitors or membrane pore-forming peptides that exhibit antibacterial and antiviral activity. On the contrary, 72% of Small Hairpins were engineered, for example as the products of phage display libraries or synthesized to examine how different amino acid residue types contribute to the β-hairpin fold (FIG. 8).

Many hairpins had disulfide bonding patterns similar to those of the members of the Small Conotoxin cluster (FIG. 11). For example, there were two members of the Small Hairpin cluster and seven members of the Small Conotoxin cluster with a CX₉C motif, where X is any amino acid residue type other than cysteine; in fact, this motif represented the full sequence for the engineered DRPs 1n0aA in the Small Hairpin cluster and 3p72B in the Small Conotoxin cluster. This result indicates that care must be taken in designing phage libraries to ensure that a scaffold based on a hairpin maintains its fold; for example, certain amino acid residue types that confer hairpin properties should not be varied. Alternatively, hairpins with fewer residues between bonded cysteines could have their full surface varied with the NNK codon, which would resemble traditional random peptide libraries that have been the focus of earlier studies [42].

Likelihood of Additional Uncharacterized DRP Folds

This Example shows that 85% of DRPs with solved structures fall into 20 fold classes, although these 20 folds only represent approximately ¼ of all known DRP folds given that 81 clusters were created overall. Thus, DRP sequences are distributed non-uniformly across known DRP folds, as has been observed with globular and membrane protein sequences in general [47]. However, the question remained whether the PDB is biased toward DRPs with certain fold classes. The initial filtering step in our protocol was intended to reduce any bias by removing redundant proteins (FIG. 1A, step i). Filtering with the 90% sequence identity threshold (instead of 100%) still resulted in 79.3% of DRPs falling in the top 20 clusters (data not shown), suggesting that the non-uniform size of the DRP clusters was not an artifact of our procedure or the DRP sample in the PDB. Notably, among the 81 clusters output by this pipeline, 43 contained only one member, suggesting that there are additional unknown folds that are assumed by a small number of DRP sequences.

Phage Display Application

20 structurally distinct peptides that can be used as scaffolds for phage display were identified. The likelihood of success in a phage display experiment is dependent on library design. DRP scaffolds offer unique challenges and opportunities in this respect. The most important design consideration is the choice of residues to vary. In certain embodiments, these residues should be located in regions that are not conserved in sequence, to decrease the probability of affecting peptide folding kinetics and stability. To this end, the degree of sequence conservation was quantified across equivalent residue positions within a cluster; the blue non-conserved regions in FIG. 4 suggest optimal surfaces to vary in phage libraries. These regions frequently occur on loops and are solvent-exposed; additionally, there is only one such surface on many of the selected cluster representatives, which results in a limited number of residue positions from which to choose for variation. Finally, if the natural binding surface of the DRP is known, the residues in this region should also be considered as candidates to vary through phage display as has been done previously [38].

Additionally, while a set of diverse scaffolds was identified, the utility of the protocol increases if the varied surfaces themselves are structurally diverse as well. This property is illustrated in FIG. 5A-C, where the selected surfaces on particular DRPs are composed of different combinations of secondary structures, including loops, helices, and sheets. It is suggested that these varied surfaces would be diverse across scaffolds even if surfaces were selected randomly on each DRP; there is little structural overlap across the full length of the scaffolds, and thus there is likely to be little overlap across subsets of the scaffolds. An exception is the α- and β-defensins, where the β-class includes an N-terminal helix not present in the α-class, with the remainder of the peptide chains being structurally similar (FIG. 4; clusters 9-10). Thus, the β-defensin varied surface could include this helix to ensure it is structurally distinct from the α-defensin surface.

These considerations were applied to design three phage libraries based on selected cluster representatives. Different secondary structures were accounted for, and regions from discontinuous surfaces were varied to increase the binding footprint (FIG. 5). No enrichment was observed from panning the IKATr1 and 1KVFr1 libraries against IL-23. This result demonstrates the drawback of relying on a single phage library to produce hits using a generic panning strategy. It is likely that none of the sequences produced through phage variation had structural complementarity to IL-23 and the phage library would thus not produce a positive result regardless of the sequence diversity sampled. In contrast, panning 1ZDCr1 resulted in a modestly potent 3 μM hit. Thus, even though the theoretical sequence diversity is similar across the three libraries, only 1ZDCr1 yielded hits in a generic panning strategy, which illustrates the value of presenting sequence diversity in different topological shapes, particularly in those that confer the favorable chemical and biological stability of DRPs.

Conclusions

As described herein, an automated protocol for clustering DRPs was developed and applied to group 1,411 peptides into 81 clusters, with 85% of those DRPs falling into only 20 most populous clusters. Given the likelihood that diverse DRP sequences assume a limited number of folds, similar to proteins as a whole, these 20 clusters appear to reflect the structure and function of the majority of DRPs found throughout nature. Constructing phage libraries comprising about 10¹⁰ sequences displayed in topologically distinct conformations (FIG. 4) and panning these libraries could result in binders that disrupt protein-protein interactions associated with disease. Collectively, these libraries sample immense chemical space displayed in well-defined discontinuous surfaces that are composed of distinct combinations of secondary structures. By binding to flat protein interfaces, peptides derived from these libraries represent a promising alternative to the traditional monoclonal antibody approaches, particular when considering their non-immunogenic character [13], protease stability [21] and potential for oral delivery [22]. The usefulness of our approach has been demonstrated by the identification of a μM binder from the initial panning of phage libraries based on only three scaffolds against the IL-23 target.

Methods

Definition of Core Terms

Native Overlap

Native overlap was defined as the fraction of Cα atoms in one DRP that were within 3.5 Å of the corresponding atoms in a second DRP following structural alignment of the first DRP to the second DRP. Thus, a native overlap of 1.0 meant that all equivalent residues across the aligned DRPs are within 3.5 Å of each other and there are no gaps in the alignment (i.e. every residue in one DRP had an equivalent in the other). Structural alignments were performed using the iterative_structure align( ) command in MODELLER version 9.10 [48]; this command implemented the SALIGN algorithm [49].

### Disulfide Distance

To align by disulfide bonds equivalent across a DRP pair, the structurally equivalent disulfide bonds were first identified. This identification first enumerated all possible mappings of disulfide bonds from the first DRP to the second. Additionally, for each mapping, it was unknown which cysteines were equivalent in an equivalent disulfide bond; therefore, all possible cysteine equivalencies are generated. Thus, if two DRPs each had three disulfide bonds, there were a total of 48 mappings enumerated (six disulfide bond mappings and eight possible cysteine equivalencies for each). Then, for each mapping, a structural alignment was performed through a least-squares superposition of the mapped cysteine Ca atoms. Following the superposition, the sum of the three-dimensional distances between all equivalent Ca atoms as well as all equivalent Sy atoms was taken as the disulfide distance for that mapping. This procedure was repeated for all mappings; the final mapping was the one with the smallest disulfide distance. If the two DRPs had a different number of disulfide bonds, then each mapping had an unmapped disulfide bond, which was not considered in the sum of equivalent distances.

### Average-Linkage Hierarchical Clustering

A canonical bottom-up, average-linkage hierarchical clustering procedure was implemented to cluster the DRPs. This procedure has been extensively described [50]. Briefly, each DRP was initialized as its own cluster, and the distances between all cluster pairs were calculated (native overlap for the initial clustering and the disulfide distance for knottin reclustering). The two clusters with the shortest average distance were merged, and the average distances between the merged cluster and all other clusters were recalculated. ‘Average linkage’ refers to calculating the average distance of all pairs of DRPs across a pair of clusters. The procedure iterated, with each step consisting of merging the pair of clusters with the shortest average distance and recalculating all distances. The iteration terminated when the shortest average distance is below some cutoff; all subtrees in the cluster hierarchy that are rooted below this cutoff were the output clusters of the algorithm (FIG. 1b).

Overview of Protocol

The PDB was searched for all protein chains with fewer than 50 amino acid residues and between one and four annotated disulfide bonds. Pairwise structural alignments of all such DRPs were computed using the SALIGN algorithm. The output of these alignments were first used to filter identical DRPs from the dataset; any DRP that had 100% sequence identity and 1.0 native overlap to another DRP was discarded. The result was the initial set of filtered DRPs that were used as input to the main pipeline. (FIG. 1A, step i).

Next, the filtered DRPs were grouped using the hierarchical clustering algorithm, using native overlap as the distance metric with a cutoff of 0.7 (FIG. 1A, step ii). This cutoff was selected manually through visualization of the resulting clusters; alternate cutoffs of 0.6 and 0.8 were also assessed and rejected. Any cluster containing four or more peptides annotated with the SCOP “knottin” fold (SCOP identifier g.3) were considered “knottin clusters”; peptides from these clusters were pooled and reclustered hierarchically, using the disulfide distance metric and imposing a cutoff of 2.0 Å. Here again, the cutoff was determined through visualization of the resulting clusters, with the cutoffs of 1.5 Å and 2.5 Å also being considered, but rejected (FIG. 1A, step iii). Together with all non-knottin clusters from the initial clustering step, these reclustered knottin clusters formed a set of intermediate clusters. These intermediate clusters were used as input to singleton post-processing steps.

Singleton Reclustering

The intermediate clusters included a number of DRPs that didn't fall into one of the 25 most populated clusters, but still had significant structural similarity to a DRP that did fall in such a cluster. Such a DRP was referred to as a ‘singleton’ for these purposes; the number 25 was chosen as a cutoff point because the number of DRPs per cluster decreases significantly for the 26^{th} most populous cluster (FIG. 3). A singleton was defined as any DRP x from a cluster not ranked in the top 25 clusters by size where there existed another cluster I that fulfilled two conditions: (1) I was ranked in the top 25 clusters by size and (2) I contained a reference DRP y that aligned to x at a native overlap above the cutoff used in the initial hierarchical clustering process. When these conditions were met, x was removed from its original cluster and added to I. This procedure was repeated twice. The first iteration used the length of the longer DRP in the denominator when calculating the native overlap, which was the same procedure used in the initial hierarchical clustering step. These singletons were referred to as ‘longer singletons’ (FIG. 1A, step iv). The resulting clusters were reranked by size and the top 25 were considered as new instances of I as above. Then, new singletons were identified in the less populated clusters, this time considering the length of the shorter DRP in the native overlap calculation. These peptides, denoted ‘shorter singletons’, were reassigned to the larger clusters, resulting in the final output of the protocol (FIG. 1A, step v).

Selection of Representative DRPs

For each of the top 20 clusters, the average native overlap value between each DRP and all other DRPs in the cluster was calculated. The peptide that had the largest average native overlap value was selected as the representative for that cluster.

Sequence Identity Calculation

For each cluster, sequence identities were calculated for all DRP pairs. For each DRP pair, the structural alignment computed by SALIGN was used to identify the structurally equivalent residues across the two DRPs. The sequence identity was calculated by dividing the number of equivalent residues having the same amino acid residue type by the number of residues in the full sequence of the longer DRP. The average sequence identity for the cluster was the average of sequence identities for the DRP pairs in the cluster.

Visualization of Structural Alignment and Sequence Similarity

For each cluster, a multiple structure alignment was performed for all DRPs using SALIGN. A multiple sequence alignment was produced based on the structure alignment and used as input to the program AL2CO [51], which quantified the overall degree of conservation at each position in an alignment. The ‘sum of pairs’ method of AL2CO was used, using the BLOSUM62 scoring matrix [52] to compare similar amino acid residue types. AL2CO calculated normalized scores at each position ranging from −2 to 2; these scores were scaled to RGB color values that could be used by the structure visualization program PyMol[53] to color individual residues; thus, each residue was colored on a RGB scale of blue [0, 0, 255] to yellow [255, 255, 0]. Commands to perform the coloring were automatically generated and saved in a PyMol script, which read the aligned structures generated by SALIGN and colored each residue for each DRP according to the degree of sequence conservation in the alignment.

Phagemid Libraries

All libraries used in phage selection were phagemid based, containing an arabinose promoter driving the expression of fusion proteins of the following form: an STII secretion signal, followed by a hemagglutinin tag, a four residue linker sequence, the peptide library, another four residue linker sequence, and the M13 gene-3 coat protein. The peptide libraries were amplified using oligonucleotides containing the variable positions encoded by NNK codons. The DNA fragments encoding the desired scaffolds were then cloned into the phagemid vector and transformed into electrocompetent E. coli XL1-Blue cells.

Selection of IL-23 Binding Peptides from Naive Peptide Phage Libraries

For library selection, IL-23 recombinant protein was immobilized on a biotinylated anti-p40 antibody (eBiosciences, C8.6, #13-7129-81) conjugated to Dynabeads® MyOne™ Streptavidin Ti (Life Technologies #65601). Approximately 1×10¹² phage particles in PBS containing 1% BSA were added to the beads with or without immobilized IL-23 protein and incubated for 1 hour at room temperature. Unbound phage particles were removed by washing the beads with PBS containing 0.05% Tween 20 (PBST). Bound phage particles were eluted from the beads with 100 mM TEA, incubated for 10 minutes at room temperature, followed by immediate neutralization with Tris base. The eluted phage particles were amplified by infecting log phase XL1-Blue. After shaking for 2 hours at 37° C., the cultures were superinfected with M13KO7 helper phage and grown for another 2 hours at 37° C. Kanamycin was added to a final concentration of 70 μg/mL, and the cultures were grown overnight at 30° C. Phage particles were harvested by first incubating the supernatant with 20% PEG 8000/NaCl solution (Teknova #P4138) for 30 minutes on ice, followed by centrifugation. The phage pellet was suspended in PBS containing 1% BSA and sterile filtered through a 0.2 μM PES filter unit. The amplified phage pool was then incubated with the immobilized target, washed, eluted and amplified as above for another 3 to 5 rounds. To ensure specific binding, all amplified phage pools were pre-incubated with biotinylated anti-IL-23p40 antibody conjugated to Dynabeads® MyOne™ Streptavidin Ti prior to the addition of the target. A successful selection requires a high enrichment ratio for target specific phage clones. The enrichment ratio was calculated by dividing the number of phage particles recovered in the presence of IL-23 by that in the absence of IL-23.

Individual clones from round 6 were analyzed by single-point phage ELISAs. Positive monovalent phage clones were identified as those that bound the antibody captured IL-23 and not the antibody. Positive clones were subjected to DNA sequencing.

Phage ELISA

To facilitate the rapid analysis of phage clones, 96 well formats for phage growth and ELISAs were used. Individual XL-1 Blue colonies harboring phagemid were picked into Growth Media (2×YT supplemented with antibiotics) in a deep 96 well plate. After overnight growth, cultures were diluted 1:20 into fresh Growth Media and grown at 37° C. until OD600 reached 0.6. Cultures were superinfected with M13KO7 helper phage and grown for another 2 hours at 37° C. Kanamycin was added to a final concentration of 70 μg/ml, and the cultures were grown overnight at 30° C. Phage supernatants were collected by centrifugation, transferred to fresh 96 well plates and used directly in single-point phage ELISA.

For phage ELISA, a 96 well Immulon® 4HBX plate (VWR #62402-959) was coated with 400 ng/well of streptavidin and incubated overnight at 4° C. The wells were washed two times with PBST, blocked with PBS containing 1% casein for 1 hour at room temperature, and washed again three times with PBST. A biotinylated anti-p40 antibody was added to each well at 250 ng/well diluted in Assay Buffer (PBS containing 0.5% casein), washed three times with PBST, followed by addition of Assay Buffer in the presence of absence of IL-23 at 50 ng/well. The plate was washed three times with PBST. Phage supernatants were added to individual wells and incubated for 1 hour at room temperature. The plate was then washed four times with PBST. The presence of phage particles was detected by incubation with a horse radish peroxidase (HRP) conjugated anti-M13 antibody (GE Healthcare #27942101) diluted 1:5000 in PBS for 1 hour at room temperature. Finally, the plate was washed three times with PBST. Signals were visualized with TMB One Component HRP Membrane Substrate (SurModics #TMBW-1000-01), quenched with 2 M sulfuric acid and read spectrophotometrically at 450 nm.

Peptide Synthesis

Peptides were synthesized using the Merrifield solid phase synthesis techniques on a 12 channel multiplex Symphony® peptide synthesizer (Protein Technologies, Inc.) and were assembled using O-Benzotriazole-N,N,N′,N′-tetramethyluroniumhexafluorophosphate (HBTU) and N,N-diisopropylethylamine (DIPEA) coupling conditions. Rink Amide MBHA resin was used for peptides with C-terminal amides and pre-loaded Wang Resin with N-α-Fmoc protected amino acids was used for peptides with C-terminal acids. The coupling reagents (HBTU and DIPEA premixed) and amino acid solutions were prepared in dimethylformamide (DMF) at a concentration of 100 mM. The peptides were assembled using standard Symphony® protocols. Pre-loaded Wang resin (250 mg, 0.14 mmol, 0.56 mmol/g loading, 100-200 mesh) or MBHA resin (250 mg, 0.15 mmol, 0.6 mmol/g loading, 100-200 mesh) was placed in each reaction vial and washed twice with 4 mL of DMF followed by 2×10 min treatments with 2.5 mL of 20% 4-methylpiperidine/DMF (conditions for Fmoc deprotection). Either the Wang resin or the Rink Amide MBHA resin was then washed three times with DMF (4 mL), followed by addition of 2.5 mL of amino acid and 2.5 mL of a HBTU-DIPEA mixture. After 45 min of reaction with frequent agitation, the resin was filtered and washed three times with DMF (4 mL). This process was then repeated.

The coupling reaction was carried out twice for the first 25 amino acids and three times for the remaining amino acids. The assembled peptide on resin was then cleaved using a 2 h treatment with cocktail reagent K[54]. The cleaved peptides were precipitated in cold (0° C.) diethyl ether, followed by washing two times with diethyl ether and air drying. The crude peptides were then submitted to an oxidation reaction in order to form the disulfide bridge. The crude peptide was dissolved in 50% acetonitrile/water at a concentration of 0.5 mg/mL. A saturated solution of iodine in methanol was added dropwise until a yellow color persisted. Excess iodine was quenched by the addition of solid ascorbic acid until the solution became colorless. The resulting solution was purified by preparative reverse-phase HPLC: Phenomenex® Luna C18 column (10 urn, 300 Å, 250×21.2 mm) using buffer A (0.1% trifluoracetic acid (TFA) in water), buffer B (0.1% TFA in acetonitrile) gradient 33%-55% buffer B over 45 min, flow rate 20 mL/min, detection at 220 nm. Fractions containing the desired product were pooled and lyophilized to give a white solid.

IL23-IL23R Competitive Binding ELISA

Immulon® 4HBX plate was coated with 200 ng/well of IL23R_huFC and incubated overnight at 4° C. The wells were washed three times with PBST, blocked with PBS containing 5% PhosphoBLOCKER (Cell Biolabs #AKR-103) for 1 hour at room temperature, and washed again three times with PBST. Serial dilutions of test peptides and IL-23 at a final concentration of 0.9 nM in PBS were added to each well, and incubated for 2 hours at room temperature. After the wells were washed, bound IL-23 was detected by incubation with 50 ng/well of goat anti-p40 polyclonal antibodies (R&D Systems #AF309) diluted in PBS for 1 hour at room temperature. The wells were again washed four times with PBST. The secondary antibodies, HRP conjugated donkey anti-goat IgG (Jackson ImmunoResearch Laboratories #705-035-147) diluted 1:5000 in PBS was then added, and incubated for 30 minutes at room temperature. The plate was finally washed as above. Signals were visualized with TMB One Component HRP Membrane Substrate, quenched with 2 M sulfuric acid and read spectrophotometrically at 450 nm.

### List of Abbreviations

DRP: Disulfide-Rich Peptide; IL-23: Interleukin-23; IL-23R: Interleukin-23 Receptor; IL-6: Interleukin-6; PDB: Protein Data Bank; SCOP: Structural Classification of Proteins; TNF: Tumor Necrosis Factor

### REFERENCES

1. Fosgerau K, Hoffmann T: Peptide therapeutics: current status and future directions. Drug Discov Today 2015, 20:122-128.
2. Rubinstein M, Niv M Y: Peptidic modulators of protein-protein interactions: Progress and challenges in computational design. Biopolymers 2009, 91:505-513.
3. Muller P Y, Milton M N: The determination and interpretation of the therapeutic index in drug development. Nat Rev Drug Discov 2012, 11:751-761.
4. Berg J M, Tymoczko J L, Stryer L: Protein Structure and Function. 2002.
5. de Vega M J P, Martin-Martinez M, González-Muñiz R: Modulation of protein-protein interactions by stabilizing/mimicking protein secondary structure elements. Curr Top Med Chem 2007, 7:33-62.
6. Tran T T, McKie J, Meutermans W D F, Boume G T, Andrews P R, Smythe M L: Topological side-chain classification of D-turns: Ideal motifs for peptidomimetic development. J Comput Aide dMol Des 2005, 19:551-566.
7. White C J, Yudin A K: Contemporary strategies for peptide macrocyclization. Nat Chem 2011, 3:509-524.
8. Meutermans W D F, Bourne G T, Golding S W, Horton D A, Campitelli M R, Craik D, Scanlon M, Smythe M L: Difficult Macrocyclizations: New Strategies for Synthesizing Highly Strained Cyclic Tetrapeptides. Org Lett 2003, 5:2711-2714.
9. Dohm M T, Kapoor R, Barron A E: Peptoids: bio-inspired polymers as potential pharmaceuticals. Curr Pharm Des 2011, 17:2732-2747.
10. Pallaghy P K, Nielsen K J, Craik D J, Norton R S: A common structural motif incorporating a cystine knot and a triple-stranded beta-sheet in toxic and inhibitory polypeptides. Protein Sci Publ Protein Soc 1994, 3:1833-1839.
11. Poth A G, Mylne J S, Grassl J, Lyons R E, Millar A H, Colgrave M L, Craik D J: Cyclotides Associate with Leaf Vasculature and Are the Products of a Novel Precursor in Petunia (Solanaceae). J Biol Chem 2012, 287:27033-27046.
12. Smith J J, Cummins T R, Alphy S, Blumenthal K M: Molecular Interactions of the Gating Modifier Toxin ProTx-II with Nav 1.5: Implied Existence of a Novel Toxin Binding Site Coupled to Activation. J Biol Chem 2007, 282:12687-12697.
13. Craik D J, Clark R J, Daly N L: Potential therapeutic applications of the cyclotides and related cystine knot mini-proteins. Expert Opin Investig Drugs 2007, 16:595-604.
14. Luckett S, Garcia R S, Barker J J, Konarev A V, Shewry P R, Clarke A R, Brady R L: High-resolution structure of a potent, cyclic proteinase inhibitor from sunflower seeds. J Mol Biol 1999, 290:525-533.
15. Lovelace E S, Armishaw C J, Colgrave M L, Wahlstrom M E, Alewood P F, Daly N L, Craik D J: Cyclic MrIA: A Stable and Potent Cyclic Conotoxin with a Novel Topological Fold that Targets the Norepinephrine Transporter. J Med Chem 2006, 49:6561-6568.
16. Batoni G, Maisetta G, Esin S, Campa M: Human beta-defensin-3: a promising antimicrobial peptide. Mini Rev Med Chem 2006, 6:1063-1073.
17. Dutton J L, Bansal P S, Hogg R C, Adams D J, Alewood P F, Craik D J: A New Level of Conotoxin Diversity, a Non-native Disulfide Bond Connectivity in-Conotoxin AuIB Reduces Structural Definition but Increases Biological Activity. J Biol Chem 2002, 277:48849-48857.
18. Chang S-G, Choi K-D, Jang S-H, Shin H-C: Role of disulfide bonds in the structure and activity of human insulin. Mol Cells 2003, 16:323-330.
19. Hartig G R S, Tran T T, Smythe M L: Intramolecular disulphide bond arrangements in nonhomologous proteins. Protein Sci Publ Protein Soc 2005, 14:474-482.
20. Fass D: Disulfide bonding in protein biophysics. Annu Rev Biophys 2012, 41:63-79.
21. Nguyen L T, Chau J K, Perry N A, de Boer L, Zaat S A J, Vogel H J: Serum Stabilities of Short Tryptophan- and Arginine-Rich Antimicrobial Peptide Analogs. PLoS ONE 2010, 5:e12684.
22. Wong C T T, Rowlands D K, Wong C-H, Lo T W C, Nguyen G K T, Li H-Y, Tam J P: Orally Active Peptidic Bradykinin B1 Receptor Antagonists Engineered from a Cyclotide Scaffold for Inflammatory Pain Treatment. Angew Chem Int Ed 2012, 51:5620-5624.
23. Clark R J, Jensen J, Nevin S T, Callaghan B P, Adams D J, Craik D J: The Engineering of an Orally Active Conotoxin for the Treatment of Neuropathic Pain. Angew Chem Int Ed 2010, 49:6545-6548.
24. de Araujo A D, Mobli M, Castro J, Harrington A M, Vetter I, Dekan Z, Muttenthaler M, Wan J, Lewis R J, King G F, Brierley S M, Alewood P F: Selenoether oxytocin analogues have analgesic properties in a mouse model of chronic abdominal pain. Nat Commun 2014, 5:3165.
25. Li J, Zhou R, He W, Xia B: Effects of recombinant human intestinal trefoil factor on trinitrobenzene sulphonic acid induced colitis in rats. Mol Biol Rep 2011, 38:4787-4792.
26. Yu R, Wang J, Li J, Wang Y, Zhang H, Chen J, Huang L, Liu X: A novel cyclopeptide from the cyclization of PACAP(1-5) with potent activity towards PAC1 attenuates STZ-induced diabetes. Peptides 2010, 31:1062-1067.
27. Wang C K, Gruber C W, Cemazar M, Siatskas C, Tagore P, Payne N, Sun G, Wang S, Bernard C C, Craik D J: Molecular Grafting onto a Stable Framework Yields Novel Cyclic Peptides for the Treatment of Multiple Sclerosis. ACS Chem Biol 2014, 9:156-163.
28. Poth A G, Chan L Y, Craik D J: Cyclotides as grafting frameworks for protein engineering and drug design applications. Biopolymers 2013, 100:480-491.
29. Lembo A J, Kurtz C B, MacDougall J E, Lavins B J, Currie M G, Fitch D A, Jeglinski B I, Johnston J M: Efficacy of Linaclotide for Patients With Chronic Constipation. Gastroenterology 2010, 138:886-895.el.
30. Rauck R, Wallace M, Leong M, Minehart M, Webster L, Charapata S, Abraham J, Buffington D, Ellis D, Kartzinel R: A Randomized, Double-Blind, Placebo-Controlled Study of Intrathecal Ziconotide in Adults with Severe Chronic Pain. J Pain Symptom Manage 2006, 31:393-406.
31. Hollander P A, Levy P, Fineman M S, Maggs D G, Shen L Z, Strobel S A, Weyer C, Kolterman O G: Pramlintide as an adjunct to insulin therapy improves long-term glycemic and weight control in patients with type 2 diabetes: a 1-year randomized controlled trial. Diabetes Care 2003, 26:784-790.
32. Tonikian R, Zhang Y, Boone C, Sidhu S S: Identifying specificity profiles for peptide recognition modules from phage-displayed peptide libraries. Nat Protoc 2007, 2:1368-1386.
33. Zoller F, Haberkom U, Mier W: Miniproteins as Phage Display-Scaffolds for Clinical Applications. Molecules 2011, 16:2467-2485.
34. Ranganath S, Bhandari A, Avitahl-Curtis N, McMahon J, Wachtel D, Zhang J, Leitheiser C, Bemier S G, Liu G, Tran T T, Celino H, Tobin J, Jung J, Zhao H, Glen K E, Graul C, Griffin A, Schairer W C, Higgins C, Reza T L, Mowe E, Rivers S, Scott S, Monreal A, Shea C, Bourne G, Coons C, Smith A, Tang K, Mandyam R A, et al.: Discovery and Characterization of a Potent Interleukin-6 Binding Peptide with Neutralizing Activity In Vivo. PloS One 2015, 10:e0141330.
35. Cheek S, Krishna S S, Grishin N V: Structural Classification of Small, Disulfide-rich Protein Domains. J Mol Biol 2006, 359:215-237.
36. Gupta A, Van Vlijmen H W T, Singh J: A classification of disulfide patterns and its relationship to protein structure and function. Protein Sci 2004, 13:2045-2058.
37. Mas J M, Aloy P, Marti-Renom M A, Oliva B, de Llorens R, Aviles F X, Querol E: Classification of protein disulphide-bridge topologies. J Comput Aided Mol Des 2001, 15:477-487.
38. Silverman A P, Levin A M, Lahti J L, Cochran J R: Engineered Cystine-Knot Peptides that Bind αvβ3 Integrin with Antibody-Like Affinities. J Mol Biol 2009, 385:1064-1075.
39. Murzin A G, Brenner S E, Hubbard T, Chothia C: SCOP: a structural classification of proteins database for the investigation of sequences and structures. J Mol Biol 1995, 247:536-540.
40. Niederreiter L, Adolph T E, Kaser A: Anti-IL-12/23 in Crohn's disease: bench and bedside. Curr Drug Targets 2013, 14:1379-1384.
41. Pan B, Li B, Russell S J, Tom J Y., Cochran A G, Fairbrother W J: Solution structure of a phage-derived peptide antagonist in complex with vascular endothelial growth factor. J Mol Biol 2002, 316:769-787.
42. Skelton N J, Russell S, de Sauvage F, Cochran A G: Amino acid determinants of f-hairpin conformation in erythropoeitin receptor agonist peptides derived from a phage display library. J Mol Biol 2002, 316:1111-1125.
43. Starovasnik M A, Braisted A C, Wells J A: Structural mimicry of a native protein by a minimized binding domain. Proc Natl Acad Sci USA 1997, 94:10080-10085.
44. Nixon A E, Sexton D J, Ladner R C: Drugs derived from phage display: From candidate identification to clinical practice. mAbs 2014, 6:73-85.
45. Thangudu R R, Manoharan M, Srinivasan N, Cadet F, Sowdhamini R, Offmann B: Analysis on conservation of disulphide bonds and their structural features in homologous protein domain families. BMC Struct Biol 2008, 8:55.
46. The UniProt Consortium: Activities at the Universal Protein Resource (UniProt). Nucleic Acids Res 2014, 42:D191-D198.
47. Orengo C A, Jones D T, Thornton J M: Protein superfamilies and domain superfolds.

Nature 1994, 372:631-634.
48. Eswar N, Webb B, Marti-Renom M A, Madhusudhan M S, Eramian D, Shen M, Pieper U, Sali A: Comparative protein structure modeling using Modeller. Curr Protoc Bioinforma 2006:5-6.
49. Braberg H, Webb B M, Tjioe E, Pieper U, Sali A, Madhusudhan M S: SALIGN: a web server for alignment of multiple protein sequences and structures. Bioinformatics 2012, 28:2072-2073.
50. D'haeseleer P: How does gene expression clustering work? Nat Biotechnol 2005, 23:1499-1501.
51. Pei J, Grishin N V: AL2C O: calculation of positional conservation in a protein sequence alignment. Bioinforma Oxf Engl 2001, 17:700-712.
52. Henikoff S, Henikoff J G: Amino acid substitution matrices from protein blocks. Proc Natl Acad Sci USA 1992, 89:10915-10919.
53. Schrodinger: The PyMOL Molecular Graphics System, Version 1.3r1. 2010.
54. King D S, Fields C G, Fields G B: A cleavage method which minimizes side reactions following Fmoc solid phase peptide synthesis. Int J Pept Protein Res 1990, 36:255-266.
55. Guharoy M, Chakrabarti P. Secondary structure based analysis and classification of biological interfaces: identification of binding motifs in protein-protein interactions. Bioinformatics. 2007; 23:1909-1918.
56. Jochim, A. L., Arora, P. S., Systematic Analysis of Helical Protein Interfaces Reveals Targets for Synthetic Inhibitors, ACS Chem. Biol., 2010, 5 (10), pp 919-923.
57. Wohlers I, Domingues F S, Klau G W: Towards optimal alignment of protein structure distance matrices. Bioinformatics 2010, 26(18)-2273-80
58. Shindyalov I N. Boume P E: Protein structure alignment by incremental combinatorial extension (CE) of the optimal path. Protein Eng 1998. 11(9):739-47.
59. Guerler A, Knapp E W: Novel protein folds and their nonsequential structural analogs. Protein Sci 2008, 17(8):1374-82.
60 L. Baldacci, M. Golfarelli, A. Lumini, S. Rizzi. Clustering techniques for protein surfaces. Bioinformatics, 39(12), 2370-2382.
61. Ranganath, S. et al.: Discovery and Characterization of a Potent Interleukin-6 Binding Peptide with Neutralizing Activity In Vivo, PLoS One 2015, 10, e0141330.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A system comprising two or more disulfide-rich peptide (DRP) scaffold libraries, wherein each of the two or more DRP scaffold libraries comprises:
(a) a plurality of DRPs comprising at least two cysteine residues capable of forming an intramolecular disulfide bond; or
(b) a plurality of polynucleotides encoding the plurality of DRPs,
wherein the plurality of DRPs of each DRP scaffold library share one or more common three-dimensional polypeptide structural feature; and the common three-dimensional polypeptide structural feature is based on structural similarity and/or disulfide bond conservation; and the disulfide bond conservation is based on a distance between disulfide bonds of about 1.5 Å to about 2.5 Å.

2-8. (canceled)

9. The system of claim 1, wherein the common three-dimensional polypeptide structural feature of each DRP scaffold library is depicted in FIG. 4.

10. The system of claim 1, wherein each of the one or more common three-dimensional polypeptide structural features is characterized as or is shared by one of the following polypeptide groups: knottin 1, knottin 2, insulin, small conotoxin, knottin 3, small hairpin, EGF-like hairpins, medium conotoxin, α-defensin, β3-defensin, large hairpin, crambin, helix-loop-helix, LDL receptor, knottin IV, PMP inhibitors, TNF receptor, large conotoxin, tryptase inhibitor, and anti-microbial peptide.

11. The system of claim 1, wherein the plurality of DRPs of each DRP scaffold library are variants of a representative DRP.

12. The system of claim 1, wherein the plurality of DRPs within each DRP scaffold library have at least 30% identity to a representative DRP amino acid sequence for each DRP scaffold library or have an average native overlap of at least 0.5 with a representative DRP amino acid sequence for each DRP scaffold library.

13. (canceled)

14. The system of claim 12, wherein the representative DRP amino acid sequence for each DRP scaffold library is an amino acid sequence shown in FIG. 8 or FIG. 9, wherein X indicates any amino acid.

15. (canceled)

16. The system of claim 12, wherein the plurality of DRPs within each DRP scaffold library comprise a sequence having at least 80% identity to a sequence shown in FIG. 8 or FIG. 9, wherein X indicates any amino acid.

17. The system of claim 1, wherein the plurality of DRPs within each of the DRP scaffold libraries have an average native overlap of less than 0.5 with the consensus DRP amino acid sequence of other DRP scaffold libraries.

18. The system of claim 12, wherein the plurality of the DRPs within each of the DRP scaffold libraries comprise one or more amino acid modifications as compared to the representative DRPs, or wherein a plurality of polynucleotides within each of the DRP scaffold libraries encode DRPs comprising one or more amino acid modifications as compared to the representative DRPs.

19. The system of claim 18, wherein the one or more amino acid modifications comprise one or more amino acid additions, deletions or substitutions.

20. The system of claim 1, wherein the libraries are surface display libraries, and wherein the plurality of DRPs of each DRP scaffold library are fused to a cell surface polypeptide.

21. The system of claim 20, wherein the cell surface polypeptide is a cell surface polypeptide of a microorganism.

22. The system of claim 21, wherein the libraries are phage display libraries or yeast display libraries, and the plurality of DRPs are fused to a polypeptide displayed on a phage cell surface.

23. (canceled)

24. The system of claim 22, wherein a plurality of the DRPs are capable of binding to a target polypeptide when expressed on the cell surface.

25. The system of claim 1, wherein the polynucleotides encode fusion polypeptides comprising each of the DRPs present in each of the DRP scaffold libraries fused to a cell surface polypeptide.

26. The system of claim 25, wherein the polynucleotides are expression vectors.

27. A method of identifying a disulfide-rich peptide (DRP) that specifically binds to a target polypeptide, comprising:
(a) contacting the target polypeptide with the system or two or more disulfide-rich peptide (DRP) scaffold libraries of claim 1; and
(b) detecting an amount of binding of the target polypeptide to a first DRP of a DRP scaffold library,
wherein if the amount of binding of the first DRP to the target polypeptide is greater than the amount of binding of the first DRP to a control polypeptide, the first DRP specifically bind to the target polypeptide.

28. (canceled)

29. A method of generating two or more disulfide-rich peptide (DRP) scaffold libraries, wherein each of the two or more DRP scaffold libraries comprises:
(i) a plurality of DRPs comprising at least two cysteine residues capable of forming an intramolecular disulfide bond; or
(ii) a plurality of polynucleotides encoding the plurality of DRPs,
wherein the plurality of DRPs of each DRP scaffold library share a common three-dimensional polypeptide structural feature, the method comprising:
(a) identifying two or more groups of DRPs comprising disulfide bonds, wherein the DRPs of each group share a different three-dimensional polypeptide structural feature;
(b) identifying a consensus DRP within each of the two or more groups of DRP, optionally wherein the peptides within each of the groups have an average native overlap of at least 0.5 with the consensus peptide of the group and/or an average native overlap of less than 0.5 with the consensus peptides of other groups;
(c) for each group of DRPs, producing a plurality of DRPs having at least 30% sequence identity to the consensus DRP of the group and comprising one or more amino acid modifications as compared to the consensus DRP,
wherein each of the plurality of DRPs constitutes a disulfide-rich DRP scaffold library.

30. The method of claim 29, wherein the plurality of peptides of (c) are fused in-frame to a cell surface polypeptide.

31. A method for identifying two or more clusters of disulfide-rich peptides (DRPs), comprising:
(a) identifying in a protein database a plurality of DRPs comprising less than 50 amino acid residues and comprising at least one disulfide bond;
(b) optionally removing duplicate DRPs from the plurality of DRPs identified in (a);
(c) clustering the plurality of DRPs into two or more clusters based on peptide structural homology;
(d) optionally reclustering knottin DRPs based on core disulfide bond structure; and
(e) optionally re-assigning DRPs in less-populated clusters to other clusters, thus identifying two or more clusters of DRPs, wherein the DRPs of each cluster share a common three-dimensional polypeptide structural feature.

32-38. (canceled)


SEQUENCE LISTING (819 sequences)

SEQ ID NO: 1 - protein, 16 aa, Artificial Sequence
  Synthetic construct
TYSCHFGPLTWVCKPQ

SEQ ID NO: 2 - protein, 34 aa, Artificial Sequence
  Synthetic construct
FNMQCQRRFYEALHDPNLNEEQRNAKIKSIRDDC

SEQ ID NO: 3 - protein, 34 aa, Homo sapiens
  misc_feature (6)..(6): Xaa can be any naturally occurring amino acid
  misc_feature (9)..(10): Xaa can be any naturally occurring amino acid
  misc_feature (13)..(14): Xaa can be any naturally occurring amino acid
  misc_feature (20)..(20): Xaa can be any naturally occurring amino acid
  misc_feature (24)..(24): Xaa can be any naturally occurring amino acid
  misc_feature (28)..(28): Xaa can be any naturally occurring amino acid
  misc_feature (31)..(31): Xaa can be any naturally occurring amino acid
FNMQCXRRXXEAXXDPNLNXEQRXAKIXSIXDDC

SEQ ID NO: 4 - protein, 19 aa, Artificial Sequence
  Synthetic construct
GGNECDIARMWEWECFERL

SEQ ID NO: 5 - skipped

SEQ ID NO: 6 - protein, 41 aa, Anopheles gambiae
ATCDLASGFGVGSSLCAAHCLVKGYRGGYCKNKICHCRDKF

SEQ ID NO: 7 - protein, 40 aa, Sarcophaga peregrina
ATCDLLSGTGINHSACAAHCLLRGNRGGYCNGKAVCVCRN

SEQ ID NO: 8 - protein, 36 aa, Leiurus quinquestriatus hebraeus
FTNVSCTTSKECWSVCQRLHNTSRGKCMNKKCRCYS

SEQ ID NO: 9 - protein, 37 aa, Mesobuthus martensii
FGLIDVKCFASSECWTACKKVTGSGQGKCQNNQCRCY

SEQ ID NO: 10 - protein, 38 aa, Androctonus mauretanicus mauretanicus
GVEINVKCSGSPQCLKPCKDAGMRFGKCMNRKCHCTPK

SEQ ID NO: 11 - protein, 37 aa, Tityus discrepans
IDTNVKCSGSSKCVKICIDRYNTRGAKCINGRCTCYP

SEQ ID NO: 12 - protein, 43 aa, Crassostrea gigas
GFGCPGNQLKCNNHCKSISCRAGYCDAATLWLRCTCTDCNGKK

SEQ ID NO: 13 - protein, 38 aa, Orthochirus scrobiculosus
GVIINVKCKISRQCLKPCKDAGMRFGKCMNGKCHCTPK

SEQ ID NO: 14 - protein, 38 aa, Orthochirus scrobiculosus
GVIINVKCKISRQCLEPCKKAGMRFGKCMNGKCHCTPK

SEQ ID NO: 15 - protein, 38 aa, Androctonus mauretanicus mauretanicus; identical to SEQ ID NO: 10

SEQ ID NO: 16 - protein, 38 aa, Pandinus imperator
DEAIRCTGTKDCYIPCRYITGCFNSRCINKSCKCYGCT

SEQ ID NO: 17 - protein, 34 aa, Heterometrus spinifer
ASCRTPKDCADPCRKETGCPYGKCMNRKCKCNRC

SEQ ID NO: 18 - protein, 34 aa, Heterometrus spinifer; identical to SEQ ID NO: 17

SEQ ID NO: 19 - protein, 37 aa, Tityus serrulatus
VFINAKCRGSPECLPKCKEAIGKAAGKCMNGKCKCYP

SEQ ID NO: 20 - protein, 36 aa, Leiurus quinquestriatus hebraeus
FTQESCTASNQCWSICKRLHNTNRGKCMNKKCRCYS

SEQ ID NO: 21 - protein, 37 aa, Mesobuthus martensii
VGINVKCKHSRQCLKPCKDAGMRFGKCTNGKCHCTPK

SEQ ID NO: 22 - protein, 38 aa, Pandinus imperator
IEAIRCGGSRDCYRPCQKRTGCPNAKCINKTCKCYGCS

SEQ ID NO: 23 - protein, 38 aa, Androctonus mauretanicus mauretanicus; identical to SEQ ID NO: 10

SEQ ID NO: 24 - protein, 36 aa, Buthus eupeus
RPTDIKCSESYQCFPVCKSRFGKTNGRCVNGFCDCF

SEQ ID NO: 25 - protein, 35 aa, Pandinus imperator
TISCTNEKQCYPHCKKETGYPNAKCMNRKCKCFGR

SEQ ID NO: 26 - protein, 39 aa, Centruroides margaritatus
TIINVKCTSPKQCLPPCKAQFGQSAGAKCMNGKCKCYPH

SEQ ID NO: 27 - protein, 36 aa, Mesobuthus martensii
FTDVKCTGSKQCWPVCKQMFGKPNGKCMNGKCRCYS

SEQ ID NO: 28 - protein, 37 aa, Mesobuthus martensii
VGINVKCKHSGQCLKPCKDAGMRFGKCINGKCDCTPK

SEQ ID NO: 29 - protein, 36 aa, Mesobuthus martensii
FTNVSCSASSQCWPVCKKLFGTYRGKCMNSKCRCYS

SEQ ID NO: 30 - protein, 39 aa, Mytilus galloprovincialis
GFGCPNNYQCHRHCKSIPGRCGGYCGGWHRLRCTCYRCG

SEQ ID NO: 31 - protein, 41 aa, Opisthacanthus madagascariensis
VHTNIPCRGTSDCYEPCEKKYNCARAKCMNRHCNCYNNCPW

SEQ ID NO: 32 - protein, 35 aa, Pandinus imperator
TISCTNPKQCYPHCKKETGYPNAKCMNRKCKCFGR

SEQ ID NO: 33 - protein, 29 aa, Mesobuthus martensii
TQCQSVRDCQQYCLTPDRCSYGTCYCKTT

SEQ ID NO: 34 - protein, 30 aa, Mesobuthus martensii
ACYSSDCRVKCVAMGFSSGKCINSKCKCYK

SEQ ID NO: 35 - protein, 25 aa, Mesobuthus martensii
ACYSSDCRVCVAMGFSSGCINSCCY

SEQ ID NO: 36 - protein, 24 aa, Artificial Sequence
  Synthetic Construct
NLHFCQLRCKSLGLLGRCATFCAC

SEQ ID NO: 37 - protein, 23 aa, Artificial Sequence
  Synthetic Construct
NLHFCQLRCKSLGLLGRCATCAC

SEQ ID NO: 38 - protein, 25 aa, Artificial Sequence
  Synthetic Construct
NLHFCQLRCKSLGLLGKCAGSFCAC

SEQ ID NO: 39 - protein, 24 aa, Artificial Sequence
  Synthetic Construct
NLHFCQLRCKSLGLLGKCAGSCAC

SEQ ID NO: 40 - protein, 27 aa, Artificial Sequence
  Synthetic Construct
CNLARCQLSCKSLGLKGGCQGSFCTCG

SEQ ID NO: 41 - protein, 47 aa, Pachyrhizus erosus
KTCENLADTFRGPCFTDGSCDDHCKNKEHLIKGRCRDDFRCWCTRNC

SEQ ID NO: 42 - protein, 44 aa, Archaeoprepona demophon
DKLIGSCVWGAVNYTSNCNAECKRRGYKGGHCGSFANVNCWCET

SEQ ID NO: 43 - protein, 39 aa, Centruroides noxius
TIINVKCTSPKQCSKPCKELYGSSAGAKCMNGKCKCYNN

SEQ ID NO: 44 - protein, 47 aa, Vigna radiata
KTCENLANTYRGPCFTTGSCDDHCKNKEHLRSGRCRDDFRCWCTRNC

SEQ ID NO: 45 - protein, 44 aa, Archaeoprepona demophon
DKLIGSCVWGAVNYTSNCRAECKRRGYKGGHCGSFANVNCWCET

SEQ ID NO: 46 - protein, 40 aa, Pseudoplectania nigrella
GFGCNGPWDEDDMQCHNHCKSIKGYKGGYCAKGGFVCKCY

SEQ ID NO: 47 - protein, 44 aa, Archaeoprepona demophon
DKLIGSCVWGAVNYTSNCNAECKRRGYKGGHCGSFLNVNCWCET

SEQ ID NO: 48 - protein, 47 aa, Nicotiana tabacum
RECKTESNTFPGICITKPPCRKACISEKFTDGHCSKILRRCLCTKPC

SEQ ID NO: 49 - protein, 46 aa, Vigna radiata
RTCMIKKEGWGKCLIDTTCAHSCKNRGYIGGNCKGMTRTCYCLVNC

SEQ ID NO: 50 - protein, 31 aa, Mesobuthus martensii
AACYSSDCRVKCVAMGFSSGKCINSKCKCYK

SEQ ID NO: 51 - protein, 31 aa, Leiurus quinquestriatus hebraeus
AFCNLRMCQLSCRSLGLLGKCIGDKCECVKH

SEQ ID NO: 52 - protein, 33 aa, Nasonia vitripennis
ACNDRDCSLDCIMKGYNTGSCVRGSCQCRRTSG

SEQ ID NO: 53 - protein, 36 aa, Pseudacanthotermes spiniger
ACNFQSCWATCQAQHSIYFRRAFCDRSQCKCVFVRG

SEQ ID NO: 54 - protein, 46 aa, Pisum sativum
KTCEHLADTYRGVCFTNASCDDHCKNKAHLISGTCHNWKCFCTQNC

SEQ ID NO: 55 - protein, 38 aa, Leiurus quinquestriatus hebraeus
GVPINVSCTGSPQCIKPCKDAGMRFGKCMNRKCHCTPK

SEQ ID NO: 56 - protein, 31 aa, Androctonus mauretanicus mauretanicus
TVCNLRRCQLSCRSLGLLGKCIGVKCECVKH

SEQ ID NO: 57 - protein, 32 aa, Centruroides noxius
AVCVYRTCDKDCKRRGYRSGKCINNACKCYPY

SEQ ID NO: 58 - protein, 40 aa, Pseudoplectania nigrella; identical to SEQ ID NO: 46

SEQ ID NO: 59 - protein, 47 aa, Hordeum vulgare
RICRRRSAGFKGPCVSNKNCAQVCMQEGWGGGNCDGPLRRCKCMRRC

SEQ ID NO: 60 - protein, 44 aa, Heliothis virescens
DKLIGSCVWGAVNYTSDCNGECLLRGYKGGHCGSFANVNCWCET

SEQ ID NO: 61 - protein, 44 aa, Heliothis virescens
DKLIGSCVWGAVNYTSDCNGECKRRGYKGGHCGSFANVNCWCET

SEQ ID NO: 62 - protein, 47 aa, Triticum aestivum
KICRRRSAGFKGPCMSNKNCAQVCQQEGWGGGNCDGPFRRCKCIRQC

SEQ ID NO: 63 - protein, 33 aa, Nasonia vitripennis
ACNDRDCSLDCIMKGYNFGKCVRGSCQCRRTSG

SEQ ID NO: 64 - protein, 44 aa, Drosophila melanogaster
DCLSGRYKGPCAVWDNETCRRVCKEEGRSSGHCSPSLKCWCEGC

SEQ ID NO: 65 - protein, 39 aa, Centruroides limbatus
TVIDVKCTSPKQCLPPCKAQFGIRAGAKCMNGKCKCYPH

SEQ ID NO: 66 - protein, 35 aa, Tityus serrulatus
VVIGQRCYRSPDCYSACKKLVGKATGKCTNGRCDC

SEQ ID NO: 67 - protein, 34 aa, Leiurus quinquestriatus hebraeus
FTNVSCTTSKEWSVCQRLHNTSRGKCMNKKRCYS

SEQ ID NO: 68 - protein, 31 aa, Leiurus quinquestriatus hebraeus
CTTSKECWSVCQRLHNTSKGWCDHRGCICES

SEQ ID NO: 69 - protein, 31 aa, Mesobuthus martensii
TPYPVNCKTDRDCVMCGLGISCKNGYCQGCT

SEQ ID NO: 70 - protein, 31 aa, Orthochirus scrobiculosus
CKISRQCLKPCKDAGMRFGKCMNGKCHCTPK

SEQ ID NO: 71 - protein, 31 aa, Mesobuthus martensii; identical to SEQ ID NO: 69

SEQ ID NO: 72 - protein, 38 aa, Androctonus mauretanicus mauretanicus; identical to SEQ ID NO: 10

SEQ ID NO: 73 - protein, 40 aa, Anopheles gambiae
ATCDLASGFGVGSSLCAAHCIARRYRGGYCNSKAVCVCRN

SEQ ID NO: 74 - protein, 39 aa, Scorpio maurus palmatus
GVPINVSCTGSKDCYAPCRKQTGCPNAKCINKSCKCYGC

SEQ ID NO: 75 - protein, 23 aa, Tityus obscurus
ACGSCRKKCKGSGKCINGRCKCY

SEQ ID NO: 76 - protein, 40 aa, Tityus serrulatus
WCSTCLDLACGASRECYDPCFKAFGRAHGKCMNNKCRCYT

SEQ ID NO: 77 - protein, 34 aa, Artificial Sequence
  Synthetic Construct
INVKCSLPQQCIKPCKDAGMRFGKCMNKKCRCYS

SEQ ID NO: 78 - protein, 33 aa, Pandinus imperator
LVKCRGTSDCGRPCQQQTGPNSKCINRMCKCYG

SEQ ID NO: 79 - protein, 31 aa, Mesobuthus martensii
TPFAIKCATDADCSRKCPGNPPCRNGFCACT

SEQ ID NO: 80 - protein, 46 aa, Homo sapiens
VSITKCSSDMNGYCLHGQCIYLVDMSQNYCRCEVGYTGVRCEHFFL

SEQ ID NO: 81 - protein, 47 aa, Homo sapiens
ECPLSHDGYCLHDGVCMYIEALDKYACNCVVGYIGERCQYRDLKWWE

SEQ ID NO: 82 - protein, 49 aa, Mus musculus
NSYPGCPSSYDGYCLNGGVCMHIESLDSYTCNCVIGYSGDRCEHADLLA

SEQ ID NO: 83 - protein, 46 aa, Homo sapiens
SDGDQCASSPCQNGGSCKDQLQSYICFCLPAFEGRNCETHKDDGSA

SEQ ID NO: 84 - protein, 46 aa, Homo sapiens; identical to SEQ ID NO: 83

SEQ ID NO: 85 - protein, 46 aa, Homo sapiens; identical to SEQ ID NO: 83

SEQ ID NO: 86 - protein, 46 aa, Homo sapiens; identical to SEQ ID NO: 83

SEQ ID NO: 87 - protein, 41 aa, Homo sapiens
SDGDQCASSPCQNGGSCKDQLQSYICFCLPAFEGRNCETHK

SEQ ID NO: 88 - protein, 43 aa, Mus musculus
PGPSSYDGYCLNGGVMHIESLDSYTCNCVIGYSGDRCQTRDLR

SEQ ID NO: 89 - protein, 41 aa, Bos taurus
KDGDQCEGHPCLNQGHCKGIGDYTCTCAEGFEGKNCEFSTR

SEQ ID NO: 90 - protein, 39 aa, Homo sapiens
VDGDQCESNPCLNGGSCKDDINSYECWCPFGFEGKNCEL

SEQ ID NO: 91 - protein, 39 aa, Homo sapiens; identical to SEQ ID NO: 90

SEQ ID NO: 92 - protein, 42 aa, Bos taurus
KDGDQCEGHPCLNQGHCKDGIGDYTCTCAEGFEGKNCEFSTR

SEQ ID NO: 93 - protein, 38 aa, Mus musculus
DVNECISNPCQNDATCLDQIGEFQCICMPGYEGVYCEI

SEQ ID NO: 94 - protein, 38 aa, Mus musculus; identical to SEQ ID NO: 93

SEQ ID NO: 95 - protein, 40 aa, Homo sapiens
TASCQDMSCSKQGECLETIGNYTCSCYPGFYGPECEYVRE

SEQ ID NO: 96 - protein, 38 aa, Mus musculus; identical to SEQ ID NO: 93

SEQ ID NO: 97 - protein, 28 aa, Hydra vulgaris
AQNPCSLQQPGCSSACAPACRLSCCSLG

SEQ ID NO: 98 - protein, 42 aa, Anthopleura elegantissima
GTACSCGNSKGIYWFYRPSCPTDRGYTGSCRYFLGTCCTPAD

SEQ ID NO: 99 - protein, 35 aa, Buthus eupeus
MCMPCFTTDPNMAKKCRDCCGGNGKCFGPQCLCNR

SEQ ID NO: 100 - protein, 36 aa, Leiurus quinquestriatus quinquestriatus
MCMPCFTTDHQMARKCDDCCGGKGRGKCYGPQCLCR

SEQ ID NO: 101 - protein, 36 aa, Artificial Sequence
  Synthetic Construct
AAAISCVGSPECPPKCRAQGCKNGKCMNRKCKCYYC

SEQ ID NO: 102 - protein, 42 aa, Centruroides noxius
DRDSCVDKSRCAKYGYYQECQDCCKNAGHNGGTCMFFKCKCA

SEQ ID NO: 103 - protein, 42 aa, Centruroides noxius; identical to SEQ ID NO: 102

SEQ ID NO: 104 - protein, 37 aa, Tityus obscurus
GSTGPQTTCQAAMCEAGCKGLGKSMESCQGDTCKCKA

SEQ ID NO: 105 - protein, 40 aa, Anopheles gambiae
ATCDLASIFNVNHALCAAHCIARRYRGGYCNSKAVCVCRN

SEQ ID NO: 106 - protein, 34 aa, Mesobuthus tamulus
GSVGCAECPMHCKGKMAKPTCENEVCKCNIGKKD

SEQ ID NO: 107 - protein, 40 aa, Protophormia terraenovae
ATCDLLSGTGINHSACAAHCLLRGNRGGYCNGKGVCVCRN

SEQ ID NO: 108 - protein, 40 aa, Anopheles gambiae
ATCDLASKWNWNHTLCAAHCIARRYRGGYCNSKAVCVCRN

SEQ ID NO: 109 - protein, 34 aa, Scorpio maurus palmatus
VSCTGSKDCYAPCRKQTGCPNAKCINKSCKCYGC

SEQ ID NO: 110 - protein, 34 aa, Heterometrus spinifer
IRCSGSRDCYSPCMKQTGCPNAKCINKSCKCYGC

SEQ ID NO: 111 - protein, 27 aa, Artificial Sequence
  Synthetic Construct
FFCPFGCALVDCGPNRPCRDTGFSCDC

SEQ ID NO: 112 - protein, 43 aa, Anopheles gambiae
ATCDLASFSSQWVTPNDSLCAAHCIARRYRGGYCNGKRVCVCR

SEQ ID NO: 113 - protein, 41 aa, Gastrophysa atrocyanea
AVRIGPCDQVCPRIVPERHECCRAHGRSGYAYCSGGGMYCN

SEQ ID NO: 114 - protein, 18 aa, Torpedo californica
YRGWKHWVYYTCCPDTPY

SEQ ID NO: 115 - protein, 16 aa, Homo sapiens
NDDCELCVNVACTGCL

SEQ ID NO: 116 - protein, 31 aa, Helianthus annuus
GYKTSISTITIEDNGRCTKSIPPICFPDGRP

SEQ ID NO: 117 - protein, 20 aa, Artificial Sequence
  Synthetic Construct
LEAIPCSIPPCFAFNKPFVF

SEQ ID NO: 118 - protein, 41 aa, Homo sapiens
ECDTINCERYNGQVCGGPGRGLCFCGKCRCHPGFEGSACQA

SEQ ID NO: 119 - protein, 24 aa, Hydra vulgaris
APMQAPVQAAPACMASCAPQCCGR

SEQ ID NO: 120 - protein, 41 aa, Ananas comosus
EEYKCYCTDTYSDCPGFCKTCKAEFGKYICLDLISPNDCVK

SEQ ID NO: 121 - protein, 35 aa, Agriosphodrus dohrni
ADDDCLPRGSKCLGENKQCCKGTTCMFYANRCVGV

SEQ ID NO: 122 - protein, 32 aa, Apomastus schlingeri
WLGCARVKEACGPWEWPCCSGLKCDGSECHPQ

SEQ ID NO: 123 - protein, 33 aa, Haplopelma hainanum
GCKGFGDSCTPGKNECCPNYACSSKHKWCKVYL

SEQ ID NO: 124 - protein, 33 aa, Haplopelma schmidti
ACKGVFDACTPGKNECCPNRVCSDKHKWCKWKL

SEQ ID NO: 125 - protein, 34 aa, Grammostola rosea
GCLEFWWKCNPNDDKCCRPKLKCSKLFKLCNFSF

SEQ ID NO: 126 - protein, 36 aa, Grammostola rosea
DCVRFWGKCSQTSDCCPHLACKSKWPRNICVWDGSV

SEQ ID NO: 127 - protein, 28 aa, Conus tulipa
SCSGRDSRCVCCMGLMCSRGKCVSIYGE

SEQ ID NO: 128 - protein, 34 aa, Grammostola rosea
ECGKFMWKCKNSNDCCKDLVCSSRWKWCVLASPF

SEQ ID NO: 129 - protein, 34 aa, Chilobrachys jingzhao
ECRKMFGGCSVDSDCCAHLGCKPTLKYCAWDGTF

SEQ ID NO: 130 - protein, 34 aa, Stromatopelma calceatum griseipes
TCRYLFGGCKTTADCCKHLACRSDGKYCAWDGTF

SEQ ID NO: 131 - protein, 32 aa, Amaranthus hypochondriacus
CIPKWNRCGPKMDGVPCCEPYTCTSDYYGNCS

SEQ ID NO: 132 - protein, 38 aa, Hadronyche infensa
KCLAEAADCSPWSGDSCCKPYLCSCIFFYPCSCRPKGW

SEQ ID NO: 133 - protein, 41 aa, Psalmopoeus cambridgei
SEDCIPKWKGCVNRHGDCCEGLECWKRRRSFEVCVPKTPKT

SEQ ID NO: 134 - protein, 42 aa, Tachypleus tridentatus
YITCLFRGARCRVYSGRSCCFGYYCRRDFPGSIFGTCSRRNF

SEQ ID NO: 135 - protein, 42 aa, Tachypleus tridentatus
YVSCLFRGARCRVYSGRSCCFGYYCRRDFPGSIFGTCSRRNF

SEQ ID NO: 136 - protein, 35 aa, Haplopelma schmidti
ECLEIFKACNPSNDQCCKSSKLVCSRKTRWCKYQI

SEQ ID NO: 137 - protein, 35 aa, Haplopelma hainanum
ECLGFGKGCNPSNDQCCKSSNLVCSRKHAWCKYEI

SEQ ID NO: 138 - protein, 25 aa, Artificial Sequence
  Synthetic Construct
CKGTGKSCSRIAYNCCTGSCRSGKC

SEQ ID NO: 139 - protein, 26 aa, Conus purpurascens
CRINQKCFQHLDDCCSRKCNRFNKCV

SEQ ID NO: 140 - protein, 35 aa, Haplopelma hainanum
ECLGFGKGCNPSNDQCCKSSNLVCSRKHRWCKYEI

SEQ ID NO: 141 - protein, 46 aa, Homo sapiens
ALAEGQSCGVYTERCAQGLRCLPRQDEEKPLHALLHGRGVCLNEKS

SEQ ID NO: 142 - protein, 33 aa, Haplopelma hainanum
ECKGFGKSCVPGKNECCSGYACNSRDKWCKVLL

SEQ ID NO: 143 - protein, 35 aa, Haplopelma hainanum
ECLGFGKGCNPSNDQCCKSSNLVCSRAHRWCKYEI

SEQ ID NO: 144 - protein, 25 aa, Conus geographus
CKSGSSCSKTSYNCCRSCNYTKRCY

SEQ ID NO: 145 - protein, 24 aa, Conus geographus
CKSGSSCSTSYNCCRSCNYTKRCY

SEQ ID NO: 146 - protein, 25 aa, Conus magus
CKGKGAKCSRLMYDCCTGSCRSGKC

SEQ ID NO: 147 - protein, 28 aa, Artificial Sequence
  Synthetic Construct
RCLPSGKACAGVTQKIPCCGSCVRGKCS

SEQ ID NO: 148 - protein, 35 aa, Geolycosa sp. (strain A267TDLS2-KZARNA)
GYCAEKGIRCDDIHCCTGLKCKCNASGYNCVCRKK

SEQ ID NO: 149 - protein, 36 aa, Hypocrea jecorina
TQSHYGQCGGIGYSGPTVCASGTTCQVLNPYYSQCL

SEQ ID NO: 150 - protein, 31 aa, Conus ermineus
DDCIKYGFCSLPILKNGLCCSGACVGVCADL

SEQ ID NO: 151 - protein, 33 aa, Pandinus imperator
GDCLPHLKRCKADNDCCGKKCKRRGTNAEKRCR

SEQ ID NO: 152 - protein, 26 aa, Conus magus
CKGKGAPCRKTMYDCCSGSCGRRGKC

SEQ ID NO: 153 - protein, 35 aa, Grammostola rosea
ECRYLFGGCKTTSDCCKHLGCKFRDKYCAWDFTFS

SEQ ID NO: 154 - protein, 35 aa, Allagelena opulenta
GGCLPHNRFCNALSGPRCCSGLKCKELSIWDSRCL

SEQ ID NO: 155 - protein, 34 aa, Momordica cochinchinensis
CPKILKKCRRDSDCPGACICRGNGYCGSGSDGGV

SEQ ID NO: 156 - protein, 36 aa, Agelenopsis aperta
ECVPENGHCRDWYDECCEGFYCSCRQPPKCICRNNN

SEQ ID NO: 157 - protein, 26 aa, Conus striatus
CKLKGQSCRKTSYDCCSGSCGRSGKC

SEQ ID NO: 158 - protein, 27 aa, Conus textile
WCKQSGEMCNLLDQNCCDGYCIVLVCT

SEQ ID NO: 159 - protein, 25 aa, Conus magus
CKGKGAKCSRLMYDCCTGSCRSGKC

SEQ ID NO: 160 - protein, 26 aa, Conus magus
CKGKGAPCRKTMYDCCSGSCGRRGKC

SEQ ID NO: 161 - protein, 25 aa, Conus magus
CKGKGAKCSKLMYDCCTGSCRSGKC

SEQ ID NO: 162 - protein, 26 aa, Conus magus
CKGKGAKCSRLMYDCCTGSCRSGKCG

SEQ ID NO: 163 - protein, 37 aa, Pireneitega luctuosa
ACVGDGQRCASWSGPYCCDGYYCSCRSMPYCRCRNNS

SEQ ID NO: 164 - protein, 36 aa, Chilobrachys jingzhao
EGECGGFWWKCGSGKPACCPKYVCSPKWGLCNFPMP

SEQ ID NO: 165 - protein, 29 aa, Paraphysa scrofa
YCQKWMWTCDSARKCCEGLVCRLWCKKII

SEQ ID NO: 166 - protein, 25 aa, Conus striatus
CKAAGKPCSRIAYNCCTGSCRSGKC

SEQ ID NO: 167 - protein, 36 aa, Chilobrachys jingzhao
DGECGGFWWKCGRGKPPCCKGYACSKTWGWCAVEAP

SEQ ID NO: 168 - protein, 33 aa, Psalmopoeus cambridgei
ACGILHDNCVYVPAQNPCCRGLQCRYGKCLVQV

SEQ ID NO: 169 - protein, 31 aa, Conus marmoreus
ACSKKWEYCIVPILGFVYCCPGLICGPFVCV

SEQ ID NO: 170 - protein, 31 aa, Conus ermineus; identical to SEQ ID NO: 150

SEQ ID NO: 171 - protein, 37 aa, Hadronyche versuta
AICTGADRPCAACCPCCPGTSCKAESNGVSYCRKDEP

SEQ ID NO: 172 - protein, 43 aa, Macrothele gigas
CGSKRAWCKEKKDCCCGYNCVYAWYNQQSSCERKWKYLFTGEC

SEQ ID NO: 173 - protein, 31 aa, Conus geographus
ACSGRGSRCQCCMGLRCGRGNPQKCIGAHDV

SEQ ID NO: 174 - protein, 34 aa, Grammostola rosea; identical to SEQ ID NO: 125

SEQ ID NO: 175 - protein, 48 aa, Agelenopsis aperta
EDNCIAEDYGKCTWGGTKCCRGRPCRCSMIGTNCECTPRLIMEGLSFA

SEQ ID NO: 176 - protein, 35 aa, Agelenopsis aperta
CIAEDYGKCTWGGTKCCRGRPCRCSMIGTNCECTP

SEQ ID NO: 177 - protein, 48 aa, Agelenopsis aperta; identical to SEQ ID NO: 175

SEQ ID NO: 178 - protein, 34 aa, Peirates turpis
AEKDCIAPGAPCFGTDKPCCNPRAWCSSYANKCL

SEQ ID NO: 179 - protein, 26 aa, Conus magus
CKGKGAPCRKTMYDCCKGRCGRRGRC

SEQ ID NO: 180 - protein, 48 aa, Agelenopsis aperta
KKKCIAKDYGRCKWGGTPCCRGRGCICSIMGTNCECKPRLIMEGLGLA

SEQ ID NO: 181 - protein, 48 aa, Agelenopsis aperta; identical to SEQ ID NO: 180

SEQ ID NO: 182 - protein, 48 aa, Agelenopsis aperta; identical to SEQ ID NO: 180

SEQ ID NO: 183 - protein, 29 aa, Macrothele gigas
GCKLTFWKCKNKKECCGWNACALGICMPR

SEQ ID NO: 184 - protein, 34 aa, Homo sapiens
CVRLHESCLGQQVPCCDPAATCYCRFFNAFCYCR

SEQ ID NO: 185 - protein, 26 aa, Conus textile
CKQADEPCDVFSLDCCTGICLGVCMW

SEQ ID NO: 186 - protein, 28 aa, Haplopelma schmidti
KCLPPGKPCYGATQKIPCCGVCSHNKCT

SEQ ID NO: 187 - protein, 38 aa, Phytolacca americana
AGCIKNGGRCNASAGPPYCCSSYCFQIAGQSYGVCKNR

SEQ ID NO: 188 - protein, 38 aa, Hadronyche infensa; identical to SEQ ID NO: 132

SEQ ID NO: 189 - protein, 25 aa, Hadronyche versuta
CIPSGQPCPYNENCCSQSCTGGRCD

SEQ ID NO: 190 - protein, 24 aa, Drosophila melanogaster
RPECVLNSDCPSNQACVNQKCRDP

SEQ ID NO: 191 - protein, 28 aa, Ecballium elaterium
GCPRLLMRCKQDSDCLAGCVCGPNGFCG

SEQ ID NO: 192 - protein, 36 aa, Hypocrea jecorina
TQSHYGQCGGIGYSGPTVCASGTTCQVLNPYASQCL

SEQ ID NO: 193 - protein, 31 aa, Grammostola rosea
YCQKWMWTCDEERKCCEGLVCRLWCKRIINM

SEQ ID NO: 194 - protein, 44 aa, Tachypleus tridentatus
YSRCQLQGFNCVVRSYGLPTIPCCRGLTCRSYFPGSTYGRCQRY

SEQ ID NO: 195 - protein, 30 aa, Heteropoda venatoria
DDCGKLFSGCDTNADCCEGYVCRLWCKLDW

SEQ ID NO: 196 - protein, 33 aa, Scorpio maurus palmatus
GDCLPHLKLCKENKDCCSKKCKRRGTNIEKRCR

SEQ ID NO: 197 - protein, 24 aa, Conus geographus
CKSGSSCSTSYNCCRSCNYTKRCY

SEQ ID NO: 198 - protein, 32 aa, Hadronyche versuta
LLACLFGNGRCSSNRDCCELTPVCKRGSCVSS

SEQ ID NO: 199 - protein, 42 aa, Atrax robustus
CAKKRNWCGKNEDCCCPMKCIYAWYNQQGSCQTTITGLFKKC

SEQ ID NO: 200 - protein, 40 aa, Psalmopoeus cambridgei
EDCIPKWKGCVNRHGDCCEGLECWKRRRSFEVCVPKTPKT

SEQ ID NO: 201 - protein, 34 aa, Chilobrachys jingzhao
GCGGLMAGCDGKSTFCCSGYNCSPTWKWCVYARP

SEQ ID NO: 202 - protein, 37 aa, Pireneitega luctuosa
GCLGEGEKCADWSGPSCCDGFYCSCRSMPYCRCRNNS

SEQ ID NO: 203 - protein, 34 aa, Gymnema sylvestre
QCVKKDELCIPYYLDCCEPLECKKVNWWDHKCIG

SEQ ID NO: 204 - protein, 42 aa, Hadronyche versuta
CAKKRNWCGKTEDCCCPMKCVYAWYNEQGSCQSTISALWKKC

SEQ ID NO: 205 - protein, 37 aa, Hadronyche versuta
SPTCIPSGQPCPYNENCCSQSCTFKENENGNTVKRCD

SEQ ID NO: 206 - protein, 39 aa, Artificial Sequence
  Synthetic Construct
GSCVPVDQPCSLNTQPCCDDATCTQERNENGHTVYYCRA

SEQ ID NO: 207 - protein, 39 aa, Solanum lycopersicum
LCNEPCSSNSDCIGITLCQFCKEKTDQYGLTYRTCNLLP

SEQ ID NO: 208 - protein, 37 aa, Solanum tuberosum
ZHADPICNKPCKTHDDCSGAWFCQACWNSARTCGPYV

SEQ ID NO: 209 - protein, 39 aa, Solanum tuberosum
EQHADPICNKPCKTHDDCSGAWFCQACWNSARTCGPYVG

SEQ ID NO: 210 - protein, 35 aa, Conus radiatus
GSFCKADEKCEYHADCCNCCLSGICASTNWILPGC

SEQ ID NO: 211 - protein, 36 aa, Acrocinus longimanus
CIKNGNGCQPNGSQGNCCSGYCHKQPGWVAGYCRRK

SEQ ID NO: 212 - protein, 32 aa, Haplopelma schmidti
GCLGDKCDYNNGCCSGYVCSRTWKWCVLAGPW

SEQ ID NO: 213 - protein, 41 aa, Homo sapiens
YGQVPMCDAGEQCAVRKGARIGKLCDCPRGTSCNSFLLKCL

SEQ ID NO: 214 - protein, 27 aa, Viola odorata
SAISCGETCFKFKCYTPRCSCSYPVCK

SEQ ID NO: 215 - protein, 36 aa, Hypocrea jecorina
TQSHYGQCGGIGYSGPTVCASGTTCQVLNPAYSQCL

SEQ ID NO: 216 - protein, 40 aa, Homo sapiens
QMFCNQTACPADCDPNTQASCECPEGYILDDGFICTDIDE

SEQ ID NO: 217 - protein, 25 aa, Androctonus australis
RSVCRQIKICRRRGGCYYKCTNRPY

SEQ ID NO: 218 - protein, 45 aa, Hadronyche versuta
LLACLFGNGRCSSNRDCCELTPVCKRGSCVSSGPGLVGGILGGIL

SEQ ID NO: 219 - protein, 21 aa, Balaenoptera physalus
GIVEQCCTSICSLYQLENYCN

SEQ ID NO: 220 - protein, 21 aa, Homo sapiens
GIVEQCCKSICSLYQLENYCN

SEQ ID NO: 221 - protein, 21 aa, Artificial Sequence
  Synthetic Construct
GIVEQCCASVCSLYQLENYCN

SEQ ID NO: 222 - protein, 21 aa, Bos taurus
GIVEQCCTSVCSLYQLENYCN

SEQ ID NO: 223 - protein, 21 aa, Physeter macrocephalus
GITEQCCTSICSLYQLENYCN

SEQ ID NO: 224 - protein, 21 aa, Sus scrofa
GIVEQCCHSICSLYQVENYCN

SEQ ID NO: 225 - protein, 21 aa, Balaenoptera physalus
GILEQCCTSICSLYQLENYCN

SEQ ID NO: 226 - protein, 21 aa, Homo sapiens
GIVEQCCHSICSLYQLENYCN

SEQ ID NO: 227 - protein, 21 aa, Homo sapiens
GIVHQCCHSICSLYQLENYCN

SEQ ID NO: 228 - protein, 21 aa, Homo sapiens
GIVEQCCTSICSLYQLENYCN

SEQ ID NO: 229 - protein, 21 aa, Homo sapiens
GIVEQCCTSICSLYQLENYCN

SEQ ID NO: 230 - protein, 22 aa, Artificial Sequence
  Synthetic Construct
RGIVEQCCTSICSLYQLENYCN

SEQ ID NO: 231 - protein, 22 aa, Homo sapiens
RGIVEQCCTSICSLYQLENYCN

SEQ ID NO: 232 - protein, 21 aa, Homo sapiens
GIVEQCCTSICSLYQLENYCN

SEQ ID NO: 233 - protein, 21 aa, Homo sapiens
GAVEQCCTSICSLYQLENYCN

SEQ ID NO: 234 - protein, 21 aa, Homo sapiens
GIGEQCCTSICSLYQLENYCN

SEQ ID NO: 235 - protein, 20 aa, Physeter macrocephalus
IVEQCCTSICSLYQLENYCN

SEQ ID NO: 236 - protein, 20 aa, Physeter macrocephalus
GVEQCCTSICSLYQLENYCN

SEQ ID NO: 237 - protein, 20 aa, Balaenoptera physalus
GIEQCCTSICSLYQLENYCN

SEQ ID NO: 238 - protein, 20 aa, Homo sapiens
GIVEQCCSICSLYQLENYCN

SEQ ID NO: 239 - protein, 19 aa, Balaenoptera physalus
IVEQCCSICSLYQLENYCN

SEQ ID NO: 240 - protein, 21 aa, Physeter macrocephalus
GIVEQCCTSICSLYQLENYCN

SEQ ID NO: 241 - protein, 20 aa, Bombyx mori
GIVDECCLRPCSVDVLLSYC

SEQ ID NO: 242 - protein, 20 aa, Balaenoptera physalus
GIVEQCCTSICSLYQLENYC

SEQ ID NO: 243 - protein, 20 aa, Homo sapiens
DLQTLCCTDGCSMTDLSALC

SEQ ID NO: 244 - protein, 21 aa, Homo sapiens
GIVEQCCTSICSLYQLENYCN

SEQ ID NO: 245 - protein, 19 aa, Balaenoptera physalus
IVEQCCTSICSLYQLENYC

SEQ ID NO: 246 - protein, 22 aa, Drosophila melanogaster
GVVDSCCRNSCSFSTLRAYCDS

SEQ ID NO: 247 - protein, 23 aa, Drosophila melanogaster
RGVVDSCCRNSCSFSTLRAYCDS

SEQ ID NO: 248 - protein, 20 aa, Homo sapiens
DLQTLCCTDGCSMTDLSALC

SEQ ID NO: 249 - protein, 23 aa, Gorilla gorilla
LYSALANKCCHVGCTKRSLARFC

SEQ ID NO: 250 - protein, 24 aa, Homo sapiens
DVLAGLSSSCCKWGCSKSEISSLC

SEQ ID NO: 251 - protein, 21 aa, Physeter macrocephalus
GIVEQCCTSICSLYQLENYCN

SEQ ID NO: 252 - protein, 21 aa, Homo sapiens
GGGEQCCTSICSLYQLENYCN

SEQ ID NO: 253 - protein, 21 aa, Homo sapiens
GIVEQCCTSICSLYQLENYCN

SEQ ID NO: 254 - protein, 21 aa, Homo sapiens
GIVEQCCTSICSLYQLENYCN

SEQ ID NO: 255 - protein, 20 aa, Homo sapiens
GIVEQCCTSICSLYQLENYC

SEQ ID NO: 256 - protein, 21 aa, Physeter macrocephalus
GIVEQCCTSICSLYQLENYCN

SEQ ID NO: 257 - protein, 27 aa, Pan troglodytes
AAATNPARYCCLSGCTQQDLLTLCPYG

SEQ ID NO: 258 - protein, 26 aa, Homo sapiens
AAATNPARYCCLSGCTQQDLLTLCPY

SEQ ID NO: 259 - protein, 26 aa, Homo sapiens
AAATNPARYCCLSGCTQQDLLTLCPY

SEQ ID NO: 260 - protein, 17 aa, Mus spretus
QCCTSICSLYQLENYCN

SEQ ID NO: 261 - protein, 17 aa, Artificial Sequence
  Synthetic Construct
CNCKAPETFLCYWRCLQ

SEQ ID NO: 262 - protein, 21 aa, Rattus norvegicus
CFGGRIDRIGAQSGLGCNSFR

SEQ ID NO: 263 - protein, 21 aa, Homo sapiens
GCFGRKMDRISSSSGLGCKVL

SEQ ID NO: 264 - protein, 18 aa, Homo sapiens
GCFGLKLDRIGSMSGLGC

SEQ ID NO: 265 - protein, 20 aa, Artificial Sequence
  Synthetic Construct
ACQAFYASSPRKSIHIGACA

SEQ ID NO: 266 - protein, 23 aa, Opisthacanthus madagascariensis
NDPCEEVCIQHTGDVKACEEACQ

SEQ ID NO: 267 - protein, 22 aa, Heterometrus fulvipes
GHACYRNCWREGNDEETCKERC

SEQ ID NO: 268 - protein, 27 aa, Opisthacanthus madagascariensis
DPCYEVCLQQHGNVKECEEACKHPVEY

SEQ ID NO: 269 - protein, 26 aa, Opisthacanthus madagascariensis
DPCYEVCLQQHGNVKECEEACKHPVE

SEQ ID NO: 270 - protein, 21 aa, Conus purpurascens
HCCLYGKCRRYGCSSASCCQR

SEQ ID NO: 271 - protein, 21 aa, Homo sapiens
CSCSSLMDKECVYFCHLDIIW

SEQ ID NO: 272 - protein, 22 aa, Conus magus
GCCSNPVCHLEHSNLCGGAAGG

SEQ ID NO: 273 - protein, 23 aa, Conus magus
GCCSNPVCHLEHSNLCGAGGAAG

SEQ ID NO: 274 - protein, 24 aa, Conus purpurascens
GCCGSYPNAACHPCSCKDRSYCGQ

SEQ ID NO: 275 - protein, 21 aa, Veronica hederifolia
CKVMCYAQRSPELLRRCLDNC

SEQ ID NO: 276 - protein, 26 aa, Veronica hederifolia
QCKVMCYAQRHSSPELLRRCLDNCEK

SEQ ID NO: 277 - protein, 13 aa, Artificial Sequence
  Synthetic Construct
ICVWQDWGAHRCT

SEQ ID NO: 278 - protein, 14 aa, Conus consors
GRCCHPACGKYYSC

SEQ ID NO: 279 - protein, 13 aa, Conus geographus
ECCNPACGRHYSC

SEQ ID NO: 280 - protein, 13 aa, Conus striatus
ICCNPACGPKYSC

SEQ ID NO: 281 - protein, 12 aa, Conus geographus
ECCPACGRHYSC

SEQ ID NO: 282 - protein, 12 aa, Conus geographus
ECCNPACGRHYC

SEQ ID NO: 283 - protein, 15 aa, Artificial Sequence
  Synthetic Construct
RCCHPQCGAAYSCRK

SEQ ID NO: 284 - protein, 12 aa, Artificial Sequence
  Synthetic Construct
CCHPQCGAAYSC

SEQ ID NO: 285 - protein, 13 aa, Artificial Sequence
  Synthetic Construct
RCCHPQCGMVEEC

SEQ ID NO: 286 - protein, 13 aa, Artificial Sequence
  Synthetic Construct
RCCHPQCGAVEEC

SEQ ID NO: 287 - protein, 13 aa, Artificial Sequence
  Synthetic Construct
RCCHPQCGMAEEC

SEQ ID NO: 288 - protein, 13 aa, Bos taurus
CRRWQWRMKKLGC

SEQ ID NO: 289 - protein, 13 aa, Conus bullatus
GCCSTPPCAVLYC

SEQ ID NO: 290 - protein, 12 aa, Conus marmoreus
VGVCCGYKLCHC

SEQ ID NO: 291 - protein, 11 aa, Artificial Sequence
  Synthetic Construct
CTERMALHNLC

SEQ ID NO: 292 - protein, 12 aa, Artificial Sequence
  Synthetic Construct
CSWRGLENHRMC

SEQ ID NO: 293 - protein, 12 aa, Artificial Sequence
  Synthetic Construct
CSARGLENHRMC

SEQ ID NO: 294 - protein, 12 aa, Conus imperialis
GCCSDPRCAWRC

SEQ ID NO: 295 - protein, 12 aa, Conus imperialis
GCCSDPRCAWEC

SEQ ID NO: 296 - protein, 12 aa, Conus imperialis
GCCSNPRCAWRC

SEQ ID NO: 297 - protein, 12 aa, Conus imperialis
GCCSDPLCAWRC

SEQ ID NO: 298 - protein, 12 aa, Conus imperialis
GCCSDPRCAWRC

SEQ ID NO: 299 - protein, 13 aa, Conus bullatus
GCCSTPPCAVLYC

SEQ ID NO: 300 - protein, 11 aa, Conus imperialis
GCCSDPRCAWR

SEQ ID NO: 301 - protein, 12 aa, Conus imperialis
GCCSDPRCAWRC

SEQ ID NO: 302 - protein, 13 aa, Artificial Sequence
  Synthetic Construct
GWCDPRWYDPFMC

SEQ ID NO: 303 - protein, 12 aa, Human immunodeficiency virus type 1 group M subtype B (isolate BRVA)
IWGCGKLICTTA

SEQ ID NO: 304 - protein, 11 aa, Artificial Sequence
  Synthetic Construct
ACDCRGDCFCG

SEQ ID NO: 305 - protein, 12 aa, Artificial Sequence
  Synthetic Construct
GCCSDARCAWRC

SEQ ID NO: 306 - protein, 10 aa, Conus marmoreus
VCCGYKLCHC

SEQ ID NO: 307 - protein, 11 aa, Artificial Sequence
  Synthetic Construct
CRVVRGDYLDC

SEQ ID NO: 308 - protein, 11 aa, Artificial Sequence
  Synthetic Construct
CKEWLSTAPCG

SEQ ID NO: 309 - protein, 11 aa, Artificial Sequence
  Synthetic Construct
CRWLRGDWRQC

SEQ ID NO: 310 - protein, 13 aa, Artificial Sequence
  Synthetic Construct
ICVVQDWGHHRCT

SEQ ID NO: 311 - protein, 13 aa, Human immunodeficiency virus type 1 group M subtype B (isolate BRVA)
IWGCSGKLICTTA

SEQ ID NO: 312 - protein, 15 aa, Bos taurus
ICLKKWPWWPWRRCK

SEQ ID NO: 313 - protein, 13 aa, Conus geographus
ECCNPACGRHYSC

SEQ ID NO: 314 - protein, 13 aa, Conus geographus
ECCNPACGRHYSC

SEQ ID NO: 315 - protein, 12 aa, Escherichia coli
CELCCNPGCAGC

SEQ ID NO: 316 - protein, 12 aa, Escherichia coli
CELCCNPLCAGC

SEQ ID NO: 317 - protein, 12 aa, Escherichia coli
CELCCNPACAGC

SEQ ID NO: 318 - protein, 13 aa, Homo sapiens
TCEICAYAACTGC

SEQ ID NO: 319 - protein, 12 aa, Artificial Sequence
  Synthetic Construct
GCCSDKRCAWRC

SEQ ID NO: 320 - protein, 17 aa, Rana grahami
YASPKCFRYPNGVLACT

SEQ ID NO: 321 - protein, 15 aa, Artificial Sequence
  Synthetic Construct
RCCHPQCGMVEECRK

SEQ ID NO: 322 - protein, 17 aa, Pseudomonas aeruginosa
KCTSDQDEQFIPKGCSK

SEQ ID NO: 323 - protein, 11 aa, Artificial Sequence
  Synthetic Construct
ACDCRGDCFCG

SEQ ID NO: 324 - protein, 10 aa, Artificial Sequence
  Synthetic Construct
VCCGYPLCHC

SEQ ID NO: 325 - protein, 13 aa, Homo sapiens
TCEICAYAACTGC

SEQ ID NO: 326 - protein, 15 aa, Bos taurus
CFRARGYGFRGPGLC

SEQ ID NO: 327 - protein, 11 aa, Conus geographus
CANPACGRHYS

SEQ ID NO: 328 - protein, 16 aa, Homo sapiens
NDDCELCVNVACTGCL

SEQ ID NO: 329 - protein, 29 aa, Cucurbita maxima
RVCPRILLECKKDSDCLAECVCLEHGYCG

SEQ ID NO: 330 - protein, 28 aa, Momordica charantia
RICPRIWMECKRDSDCMAECICVMGHCG

SEQ ID NO: 331 - protein, 29 aa, Cucurbita maxima
RVCPRILLECKKDSDCLAECVCLEHGYCG

SEQ ID NO: 332 - protein, 29 aa, Cucurbita maxima
RVCPRILMECKKDSDCLAECVCLEHGYCG

SEQ ID NO: 333 - protein, 29 aa, Cucurbita pepo
RVCPKILMECKKDSDCLAECICLEHGYCG

SEQ ID NO: 334 - protein, 28 aa, Momordica charantia
RICPRIWMECTRDSDCMAKCICVAGHCG

SEQ ID NO: 335 - protein, 29 aa, Momordica cochinchinensis
GVCPKILKKCRRDSDCPGACICRGNGYCG

SEQ ID NO: 336 - protein, 28 aa, Artificial Sequence
  Synthetic Construct
PCTLEYMRCKQDSDCLAGCVCGPNGFCG

SEQ ID NO: 337 - protein, 28 aa, Ecballium elaterium
GCPRILMRCKQDSDCLAGCVCGPNGFCG

SEQ ID NO: 338 - protein, 30 aa, Ecballium elaterium
GCPRILIRCKQDSDCLAGCVCTNNKFCGSP

SEQ ID NO: 339 - protein, 31 aa, Ecballium elaterium
GCPRILIRCKQDSDCLAGCVCGPNGFCGSPA

SEQ ID NO: 340 - protein, 30 aa, Ecballium elaterium
GCPRILIRCKQDSDCLAGCVCGPNGFCGSP

SEQ ID NO: 341 - protein, 34 aa, Momordica cochinchinensis
SGSDGGVCPKILKKCRRDSDCPGACICRGNGYCG

SEQ ID NO: 342 - protein, 34 aa, Momordica cochinchinensis; identical to SEQ ID NO: 341

SEQ ID NO: 343 - protein, 27 aa, Conus gloriamaris
SCNNSCQSHSDCASHCICTFRGCGAVN

SEQ ID NO: 344 - protein, 29 aa, Oldenlandia affinis
GLPVCGETCVGGTCNTPGCTCSWPVCTRN

SEQ ID NO: 345 - protein, 29 aa, Oldenlandia affinis
AGETCVGGTCNTPGATCSWPVCTRNGLPV

SEQ ID NO: 346 - protein, 31 aa, Viola hederacea
CGESCAMISFCFTEVIGCSCKNKVCYLNSIS

SEQ ID NO: 347 - protein, 30 aa, Viola odorata
GIPCGESCVWIPCISSAIGCSCKSKVCYRN

SEQ ID NO: 348 - protein, 29 aa, Oldenlandia affinis
CGETCVGGTCNTPGCTCSWDKCTRNGLPV

SEQ ID NO: 349 - protein, 31 aa, Chassalia parviflora
CGESCVFIPCISTLLGCSCKNKVCYRNGVIP

SEQ ID NO: 350 - protein, 29 aa, Oldenlandia affinis
CGETCVGGTCNTPGCTCSKNKCTRNGLPV

SEQ ID NO: 351 - protein, 29 aa, Oldenlandia affinis
CGETCTLGTCYTQGCTCSWPICKRNGLPV

SEQ ID NO: 352 - protein, 24 aa, Oldenlandia affinis
VCGETCVGGTCNTPGCTCSWPVCT

SEQ ID NO: 353 - protein, 30 aa, Viola odorata
CAESCVYIPCTVTALLGCSCSNRVCYNGIP

SEQ ID NO: 354 - protein, 29 aa, Oldenlandia affinis
CGETCVGGTCNTPGCTCSWPVCTRNGLPV

SEQ ID NO: 355 - protein, 29 aa, Viola arvensis
CGETCTLGTCYTAGCSCSWPVCTRNGVPI

SEQ ID NO: 356 - protein, 30 aa, Viola hederacea
CGETCFTGTCYTNGCTCDPWPVCTRNGLPV

SEQ ID NO: 357 - protein, 29 aa, Viola odorata
GIPCGSCVWIPCISSAIGCSCKSKVCYRN

SEQ ID NO: 358 - protein, 29 aa, Oldenlandia affinis
CGETCFGGTCNTPGCSCTWPICTRDGLPV

SEQ ID NO: 359 - protein, 31 aa, Viola odorata
GSIPACGESCFKGKCYTPGCSCSKYPLCAKN

SEQ ID NO: 360 - protein, 29 aa, Viola arvensis
GVPICGETCTLGTCYTAGCSCSWPVCTRN

SEQ ID NO: 361 - protein, 30 aa, Oldenlandia affinis
GTPCGESCVYIPCISGVIGCSCTDKVCYLN

SEQ ID NO: 362 - protein, 30 aa, Viola hederacea
CAESCVWIPCTVTALLGCSCSNKVCYNGIP

SEQ ID NO: 363 - protein, 28 aa, Oldenlandia affinis
GSLCGDTCFVLGCNDSSCSCNYPICVKD

SEQ ID NO: 364 - protein, 31 aa, Oldenlandia affinis
CGETCLLGTCYTTGCTCNKYRVCTKDGSVLN

SEQ ID NO: 365 - protein, 33 aa, Viola arvensis
CGESCFLGTCYTKGCSCGEWKLCYGTNGGTIFD

SEQ ID NO: 366 - protein, 30 aa, Chassalia parviflora
CGESCVWIPCISAALGCSCKNKVCYRNGIP

SEQ ID NO: 367 - protein, 36 aa, Hypocrea jecorina
TQSHAGQCGGIGYSGPTVCASGTTCQVLNPYYSQCL

SEQ ID NO: 368 - protein, 37 aa, Palicourea condensata
TFCGETCRVIPVCTYSAALGCTCDDRSDGLCKRNGDP

SEQ ID NO: 369 - protein, 30 aa, Viola odorata
SCVYIPCTVTALLGCSCSNRVCYNGIPCAE

SEQ ID NO: 370 - protein, 28 aa, Ecballium elaterium
GCPRILMRCKQDSDCLAGCVCGPNGFCG

SEQ ID NO: 371 - protein, 37 aa, Pisum sativum
ASCNGVCSPFEMPPCGTSACRCIPVGLVIGYCRNPSG

SEQ ID NO: 372 - protein, 37 aa, Glycine max
ADCNGACSPFEVPPCRSRDCRCVPIGLFVGFCIHPTG

SEQ ID NO: 373 - protein, 33 aa, Momordica cochinchinensis
GCEGKQCGLFRSCGGGCRCWPTVTPGVGICSSS

SEQ ID NO: 374 - protein, 29 aa, Oldenlandia affinis
NGLPVCGETCVGGTCNTPGCTCSWPVCTR

SEQ ID NO: 375 - protein, 29 aa, Oldenlandia affinis
SWPVCTRNGLPVCGETCVGGTCNTPGCTC

SEQ ID NO: 376 - protein, 27 aa, Human immunodeficiency virus type 2 subtype A (isolate Ghana-1)
RAPRRQGCWKCGKTGHVMAKCPERQAG

SEQ ID NO: 377 - protein, 16 aa, Artificial Sequence
  Synthetic Construct
TYSCHFGPLTWVCKPQ

SEQ ID NO: 378 - protein, 17 aa, Tachypleus tridentatus
KWCFRVCYRGICYRRCR

SEQ ID NO: 379 - protein, 18 aa, Sus scrofa
RGGRLCYCRRRFCVCVGR

SEQ ID NO: 380 - protein, 18 aa, Sus scrofa
RGGRLCYCRRRFCVCVGR

SEQ ID NO: 381 - protein, 17 aa, Tachypleus tridentatus
KWCFRVCYRGICYRRCR

SEQ ID NO: 382 - protein, 17 aa, Acanthoscurria gomesiana
CRRLCYKQRCVTYCRGR

SEQ ID NO: 383 - protein, 18 aa, Artificial Sequence
  Synthetic Construct
GFCRCLCRRGVCRCICTR

SEQ ID NO: 384 - protein, 18 aa, Limulus polyphemus
RRWCFRVCYRGFCYRKCR

SEQ ID NO: 385 - protein, 19 aa, Limulus polyphemus
RRWCFRVCYRGRFCYRKCR

SEQ ID NO: 386 - protein, 19 aa, Limulus polyphemus
RRWCFRVCYRGRFCYRKCR

SEQ ID NO: 387 - protein, 18 aa, Artificial Sequence
  Synthetic Construct
RRICRCICGRGICRCICG

SEQ ID NO: 388 - protein, 16 aa, Artificial Sequence
  Synthetic Construct
TYSCHFGPLTWVCKPQ

SEQ ID NO: 389 - protein, 17 aa, Artificial Sequence
  Synthetic Construct
RVRCRQRKGRRICIRIP

SEQ ID NO: 390 - protein, 17 aa, Tachypleus tridentatus
KWCFRVCYRGICYRRCR

SEQ ID NO: 391 - protein, 14 aa, Helianthus annuus
GRCTKSIPPICFPD

SEQ ID NO: 392 - protein, 15 aa, Artificial Sequence
  Synthetic Construct
TSCHFGPLTWVCKPQ

SEQ ID NO: 393 - protein, 13 aa, Artificial Sequence
  Synthetic Construct
DCAWHLGELVWCT

SEQ ID NO: 394 - protein, 14 aa, Helianthus annuus
GRCTKSIPPICFPD

SEQ ID NO: 395 - protein, 14 aa, Rana grahami
GCWTKSIPPKPCFG

SEQ ID NO: 396 - protein, 14 aa, Conus marmoreus
NGVCCGYKLCHCAG

SEQ ID NO: 397 - protein, 15 aa, Vigna radiata var. radiata
SCRCTKSIPPQCHCA

SEQ ID NO: 398 - protein, 15 aa, Artificial Sequence
  Synthetic Construct
NLPRCTEGPWGWVCM

SEQ ID NO: 399 - protein, 16 aa, Homo sapiens
CGADSYEMEEDGVRKC

SEQ ID NO: 400 - protein, 16 aa, Homo sapiens
CGADSYEMEEDGVRKC

SEQ ID NO: 401 - protein, 16 aa, Artificial Sequence
  Synthetic Construct
CTKSIPPICTKSIPPI

SEQ ID NO: 402 - protein, 22 aa, Homo sapiens
HFPICIFCCGCCHRSKCGMCCK

SEQ ID NO: 403 - protein, 21 aa, Arenicola marina
RWCVYAYVRVRGVLVRYRRCW

SEQ ID NO: 404 - protein, 21 aa, Arenicola marina
RWCVYAYVRIRGVLVRYRRCW

SEQ ID NO: 405 - protein, 19 aa, Gallus gallus
IPGKRTESFYECCKEPYPD

SEQ ID NO: 406 - protein, 25 aa, Bos taurus
FKCRRWQWRMKKLGAPSITCVRRAF

SEQ ID NO: 407 - protein, 27 aa, Artificial Sequence
  Synthetic Construct
SKYEYTISYTFRGPGCPTVKPVTIRCE

SEQ ID NO: 408 - protein, 27 aa, Artificial Sequence
  Synthetic Construct
SKYEYTISYTFRGPGCPTLKPITVRCE

SEQ ID NO: 409 - protein, 27 aa, Artificial Sequence
  Synthetic Construct
SKYEYTVSYTFRGPGCPTVKPISLRCE

SEQ ID NO: 410 - protein, 30 aa, Homo sapiens
EGAQWNCTACTFLNHPALIRCEQCEMPRHF

SEQ ID NO: 411 - protein, 29 aa, Human immunodeficiency virus type 2 subtype A (isolate Ghana-1)
AQQRKVIRCWNCGKEGHSARQCRAPRRQG

SEQ ID NO: 412 - protein, 25 aa, Homo sapiens
DTHFPICIFCCGCCHRSKCGMCCKT

SEQ ID NO: 413 - protein, 32 aa, Pseudaletia separata
SVQILRCPDGMQMLRSGQCVATTEPPFDPDSY

SEQ ID NO: 414 - protein, 28 aa, Nicotiana alata
RICTNCCAGKKGCKYFSDDGTFICEGES

SEQ ID NO: 415 - protein, 31 aa, Hydra vulgaris
GSQITGTCPSGCSGDCYPECPPGCCGQVNLN

SEQ ID NO: 416 - protein, 30 aa, Thermus thermophilus (strain HB27 / ATCC BAA-163 / DSM 7039)
VPARIICGCGNVIETYSTKPEIYVEVCSKC

SEQ ID NO: 417 - protein, 25 aa, Torpedo marmorata
EERGWKHWVYYTCCPDTPYLDITEE

SEQ ID NO: 418 - protein, 27 aa, Anemonia sulcata
RSCCPCYWGGCPWGQNCYPEGCSGPKV

SEQ ID NO: 419 - protein, 17 aa, Artificial Sequence
  Synthetic Construct
ACPPSHVLDMRSGTCLA

SEQ ID NO: 420 - protein, 18 aa, Artificial Sequence
  Synthetic Construct
ACPPSHVLDMRSGTCLAA

SEQ ID NO: 421 - protein, 19 aa, Artificial Sequence
  Synthetic Construct
ACPPSHVLDMRSGTCLAAE

SEQ ID NO: 422 - protein, 20 aa, Artificial Sequence
  Synthetic Construct
ACPPSHVLDMRSGTCLAAEG

SEQ ID NO: 423 - protein, 19 aa, Homo sapiens
VCAEGFAPIPGEPHRCQLF

SEQ ID NO: 424 - protein, 18 aa, Homo sapiens
CPEGYILDDGFICTDIDE

SEQ ID NO: 425 - protein, 17 aa, Homo sapiens
CPEGYILDDGFCTDIDE

SEQ ID NO: 426 - protein, 23 aa, Pseudoplusia includens
ENFNGGCLAGYMRTADGRCKPTF

SEQ ID NO: 427 - protein, 23 aa, Pseudaletia separata
ENFSGGCVAGYMRTPDGRCKPTF

SEQ ID NO: 428 - protein, 23 aa, Spodoptera litura
ENFAAGCATGYQRTADGRCKPTF

SEQ ID NO: 429 - protein, 23 aa, Manduca sexta
ENFAGGCATGFMRTADGRCKPTF

SEQ ID NO: 430 - protein, 23 aa, Mamestra brassicae
ENFAGGCLTGFMRTPDGRCKPTF

SEQ ID NO: 431 - protein, 23 aa, Bombyx mori
ENFVGGCATGFKRTADGRCKPTF

SEQ ID NO: 432 - protein, 23 aa, Manduca sexta
ENFAGGCATGYLRTADGRCKPTF

SEQ ID NO: 433 - protein, 27 aa, Hydra vulgaris
GSSSCPQFPSCSPSCAPQCSQQCCQQP

SEQ ID NO: 434 - protein, 21 aa, Hydra sp.
PCGSYCPSVCAPACAPVCCYP

SEQ ID NO: 435 - protein, 21 aa, Podisus maculiventris
GSKKPVPIIYCNRRTGKCQRM

SEQ ID NO: 436 - protein, 19 aa, Gallus gallus
IPGKRTESFYECCKEPYPD

SEQ ID NO: 437 - protein, 24 aa, Homo sapiens
PTPCVPAECFDLLVRHCVACGLLR

SEQ ID NO: 438 - protein, 21 aa, Homo sapiens
CSQNEYFDSLLHACIPCQLRC

SEQ ID NO: 439 - protein, 21 aa, Morone chrysops x Morone saxatilis
GCRFCCNCCPNMSGCGVCCRF

SEQ ID NO: 440 - protein, 20 aa, Homo sapiens
ICIFCCGCCHRSKCGMCCKT

SEQ ID NO: 441 - protein, 19 aa, Artificial Sequence
  Synthetic Construct
GLPWGCPSDIPGWNTPWAC

SEQ ID NO: 442 - protein, 28 aa, Pseudaletia separata
ENFSGGCVAGYMRTPDGRCKPTFYQLIT

SEQ ID NO: 443 - protein, 28 aa, Pseudaletia separata
ENFSGGCVAGYMRTPDGRCKPTFYQLIT

SEQ ID NO: 444 - protein, 25 aa, Pseudaletia separata
ENFSGGCVAGYMRTPDGRCKPTFYQ

SEQ ID NO: 445 - protein, 22 aa, Vigna radiata var. radiata
EPCCDSCRCTKSIPPQCHCANI

SEQ ID NO: 446 - protein, 37 aa, Leuconostoc gelidum
KYYGNGVHCTKSGCSVNWGEAFSAGVHRLANGGNGFW

SEQ ID NO: 447 - protein, 37 aa, Leuconostoc gelidum; identical to SEQ ID NO: 446

SEQ ID NO: 448 - protein, 25 aa, Hydra vulgaris
TCPSGCSGDCYPECKPGCCGQVNLN

SEQ ID NO: 449 - protein, 25 aa, Homo sapiens
DTHFPICIFCCGCCHRSKCGMCCKT

SEQ ID NO: 450 - protein, 39 aa, Brachypelma ruhnaui
IFECVFSCDIKKEGKPCKPKGEKKCTGGWRCKIKLCLKI

SEQ ID NO: 451 - protein, 33 aa, Stoichactis helianthus
RSIDTIPKSRCTAFQCKHSAKYRLSFCRKTCGT

SEQ ID NO: 452 - protein, 36 aa, Oryctes rhinoceros
ELPKLPDDKVLIRSRSNCPKGKVWNGFDCKSPFAFS

SEQ ID NO: 453 - protein, 29 aa, Human immunodeficiency virus type 2 subtype A (isolate Ghana-1)
AQQRKVIRCWACGKEGHSARQCRAPRRQG

SEQ ID NO: 454 - protein, 37 aa, Androctonus mauretanicus mauretanicus
GVEINVKCSGSPQCLKPCKDAGMRFGKCMNRKCHCTP

SEQ ID NO: 455 - protein, 37 aa, Leuconostoc gelidum; identical to SEQ ID NO: 446

SEQ ID NO: 456 - protein, 37 aa, Haplopelma schmidti
LFECSFSCEIEKEGDKPCKKKKCKGGWKCKFNMCVKV

SEQ ID NO: 457 - protein, 39 aa, Macrobdella decora
APRLPQCQGDDQEKCLCNKDECPPGQCRFPRGDADPYCE

SEQ ID NO: 458 - protein, 15 aa, Conus aulicus
GCCSYPPCFATNPDC

SEQ ID NO: 459 - protein, 16 aa, Conus episcopatus
GCCSDPRCNMNNPDYC

SEQ ID NO: 460 - protein, 16 aa, Conus pennaceus
GCCSLPPCAANNPDYC

SEQ ID NO: 461 - protein, 16 aa, Conus magus
GCCSNPVCHLEHSNLC

SEQ ID NO: 462 - protein, 16 aa, Artificial Sequence
  Synthetic Construct
GCCSRPPCILNNPDLC

SEQ ID NO: 463 - protein, 16 aa, Conus pennaceus
GCCSLPPCALNNPKYC

SEQ ID NO: 464 - protein, 16 aa, Conus pennaceus
GCCSLPPCALSNPDYC

SEQ ID NO: 465 - protein, 16 aa, Conus geographus
GCCSHPACAGNNQHIC

SEQ ID NO: 466 - protein, 16 aa, Conus victoriae
GCCSDPRCNYDHPEIC

SEQ ID NO: 467 - protein, 17 aa, Conus omaria
GCCSHPACNVNNPHICG

SEQ ID NO: 468 - protein, 19 aa, Conus tulipa
FNWRCCLIPACRRNHKKFC

SEQ ID NO: 469 - protein, 18 aa, Conus purpurascens
RDPCCSNPVCTVHNPQIC

SEQ ID NO: 470 - protein, 17 aa, Conus ermineus
RDCCYHPTCNMSNPQIC

SEQ ID NO: 471 - protein, 18 aa, Artificial Sequence
  Synthetic Construct
ALCDDPRVDRWYCQFVEG

SEQ ID NO: 472 - protein, 18 aa, Artificial Sequence
  Synthetic Construct
ALCDDPRVDRWYCQFVEG

SEQ ID NO: 473 - protein, 16 aa, Artificial Sequence
  Synthetic Construct
SGWCDPRWYDPFMCEH

SEQ ID NO: 474 - protein, 16 aa, Conus marmoreus
VCCPFGGCHELCYCCD

SEQ ID NO: 475 - protein, 17 aa, Pseudomonas aeruginosa
SCATTVDAKFRPNGCTD

SEQ ID NO: 476 - protein, 17 aa, Pseudomonas aeruginosa
SCATTVDAKFRPNGCTD

SEQ ID NO: 477 - protein, 17 aa, Homo sapiens
CSCSSLMDKECVYFCHL

SEQ ID NO: 478 - protein, 18 aa, Homo sapiens
KRCSCSSLMDKECVYFCH

SEQ ID NO: 479 - protein, 16 aa, Human respiratory syncytial virus A (strain Long)
PCSICSNNPTCWAICK

SEQ ID NO: 480 - protein, 16 aa, Human respiratory syncytial virus A (strain Long)
PCSICSNNPTCWAICK

SEQ ID NO: 481 - protein, 16 aa, Artificial Sequence
  Synthetic Construct
RNCFESVAALRRCMYG

SEQ ID NO: 482 - protein, 15 aa, Artificial Sequence
  Synthetic Construct
CRAGPLQWLCEKYFG

SEQ ID NO: 483 - protein, 21 aa, Homo sapiens
SCFGGRMDRIGAQSGLGCNSF

SEQ ID NO: 484 - protein, 19 aa, Bovine respiratory syncytial virus (strain Copenhagen)
VPCSTCEGNLACLSLCHIE

SEQ ID NO: 485 - protein, 21 aa, Conus stercusmuscarum
RCCNGRRGCSSRWCRDHSRCC

SEQ ID NO: 486 - protein, 17 aa, Conus geographus
IRDCCSNPACRVNNHVC

SEQ ID NO: 487 - protein, 17 aa, Tachypleus tridentatus
KWCFRVCYRGICYRRCR

SEQ ID NO: 488 - protein, 17 aa, Pseudomonas aeruginosa
KCTSDQDEQFIPKGCSK

SEQ ID NO: 489 - protein, 17 aa, Pseudomonas aeruginosa
KCTSDQDEQFIPKGCSK

SEQ ID NO: 490 - protein, 18 aa, Torpedo californica
YRGWKHWVYYTCCPDTPY

SEQ ID NO: 491 - protein, 15 aa, Artificial Sequence
  Synthetic Construct
EEWEVLCWTWETCER

SEQ ID NO: 492 - protein, 21 aa, Homo sapiens
CSCSSLMDKECVYFCHLDIIW

SEQ ID NO: 493 - protein, 27 aa, Homo sapiens
ACYCRIPACIAGEYGTCIYQGLWAFCC

SEQ ID NO: 494 - protein, 27 aa, Homo sapiens
DCYCRIPACIAGEYGTCIYQGLWAFCC

SEQ ID NO: 495 - protein, 30 aa, Homo sapiens
ACYCRIPACIAGERRAGTCIYQGRLWAFCC

SEQ ID NO: 496 - protein, 30 aa, Homo sapiens
DCYCRIPACIAGERRYGTCIYQGRLWAFCC

SEQ ID NO: 497 - protein, 30 aa, Homo sapiens
DCYCRIPACIAGEKKYGTCIYQGKLWAFCC

SEQ ID NO: 498 - protein, 30 aa, Homo sapiens
ACYCRIPACIAGERRYGTCIAQGRLWAFCC

SEQ ID NO: 499 - protein, 30 aa, Homo sapiens
ACYCRIPACIAGERRYGTCIYQGRLWAACC

SEQ ID NO: 500 - protein, 30 aa, Homo sapiens
ACYCRIPACIAGERRYGTCIYQGRLWAFCC

SEQ ID NO: 501 - protein, 30 aa, Homo sapiens
ACYCRAPACIAGERRYGTCIYQGRLWAFCC

SEQ ID NO: 502 - protein, 30 aa, Homo sapiens
ACYCRIPACIAGERRYGTCIYAGRLWAFCC

SEQ ID NO: 503 - protein, 30 aa, Homo sapiens
ACYCRIPACIAGERRYGTCIYQGALWAFCC

SEQ ID NO: 504 - protein, 31 aa, Homo sapiens
VCSCRLVFCRRTELRVGNCLIGGVSFTYCCT

SEQ ID NO: 505 - protein, 29 aa, Homo sapiens
ACYCRIPACIAGERRYGTCIYQGRLAFCC

SEQ ID NO: 506 - protein, 28 aa, Homo sapiens
CYCRIPACIAGERRYTCIYQGRLWAFCC

SEQ ID NO: 507 - protein, 29 aa, Homo sapiens
ACYCRIPACIAGERRYGTCYQGRLWAFCC

SEQ ID NO: 508 - protein, 32 aa, Homo sapiens
ATCYCRTGRCATRESLSGVCEISGRLYRLCCR

SEQ ID NO: 509 - protein, 32 aa, Homo sapiens
ATCYCRTGRCATHESLSGVCEISGRLYRLCCR

SEQ ID NO: 510 - protein, 32 aa, Macaca mulatta
RRTCHCRSRCLRRESNSGSCNINGRIFSLCCR

SEQ ID NO: 511 - protein, 32 aa, Homo sapiens
AFTCHCRRSCYSTEYSYGTCTVMGINHRFCCL

SEQ ID NO: 512 - protein, 32 aa, Homo sapiens; identical to SEQ ID NO: 511

SEQ ID NO: 513 - protein, 33 aa, Homo sapiens
VCSCRLVFCRRTELRVGNCLIGGVSFTYCCTRV

SEQ ID NO: 514 - protein, 33 aa, Homo sapiens; identical to SEQ ID NO: 513

SEQ ID NO: 515 - protein, 33 aa, Homo sapiens
CGGACYCRIPACIAGERRYGTCIYQGRLWAFCC

SEQ ID NO: 516 - protein, 33 aa, Homo sapiens; identical to SEQ ID NO: 515

SEQ ID NO: 517 - protein, 32 aa, Mus musculus
GLLCYCRKGHCKRGERVRGTCGIRFLYCCPRR

SEQ ID NO: 518 - protein, 32 aa, Mus musculus; identical to SEQ ID NO: 517

SEQ ID NO: 519 - protein, 32 aa, Mus musculus
GLLCYCRKGHCKRGDRVRGTCGIRFLYCCPRR

SEQ ID NO: 520 - protein, 32 aa, Oryctolagus cuniculus
MPCSCKKYCDPWEVIDGSCGLFNSKYICCREK

SEQ ID NO: 521 - protein, 25 aa, Homo sapiens
ATCYCRTGRLSGVCEISGRLYRLCC

SEQ ID NO: 522 - protein, 39 aa, Rhipicephalus bursa
LTGCKGKGGECNPLDRQCKELQAESASCGKGQKCCVWLH

SEQ ID NO: 523 - protein, 36 aa, Homo sapiens
DHYNCVSSGGQCLYSACPIFTKIEGTCYRGKAKCCK

SEQ ID NO: 524 - protein, 36 aa, Homo sapiens
DHYACVSSGGQCLYSACPIFTKIQGTCYRGKAKCCK

SEQ ID NO: 525 - protein, 36 aa, Homo sapiens
DHYNCVSSGGQCLYSACPIFTEIQGTCYRGKAKCCK

SEQ ID NO: 526 - protein, 36 aa, Homo sapiens
DHYNCVSSGGQCLYSACPIFTKIAGTCYRGKAKCCK

SEQ ID NO: 527 - protein, 36 aa, Homo sapiens
DHYNCVSSGGQCLYSACPIFTKIQGTCYRGAAKCCK

SEQ ID NO: 528 - protein, 36 aa, Homo sapiens
DHYNCVSAGGQCLYSACPIFTKIQGTCYRGKAKCCK

SEQ ID NO: 529 - protein, 36 aa, Homo sapiens
DHYNCVSSGGACLYSACPIFTKIQGTCYRGKAKCCK

SEQ ID NO: 530 - protein, 36 aa, Homo sapiens
DHYNCVSSGGQCAYSACPIFTKIQGTCYRGKAKCCK

SEQ ID NO: 531 - protein, 36 aa, Homo sapiens
DHYNCVSSGGQCLYSACPIFTKIQGTCYRGKAKCCK

SEQ ID NO: 532 - protein, 36 aa, Homo sapiens
DHYNCVSSGGQCLYSACPIFTRIQGTCYRGRARCCR

SEQ ID NO: 533 - protein, 36 aa, Homo sapiens
DHYNCVSSGGQCEYSACPIFTKIQGTCYRGKAKCCK

SEQ ID NO: 534 - protein, 36 aa, Homo sapiens
DHYNCVSSGGQCLYSACPIFTKIQGTCYRGEAKCCK

SEQ ID NO: 535 - protein, 36 aa, Homo sapiens; identical to SEQ ID NO: 531

SEQ ID NO: 536 - protein, 37 aa, Homo sapiens
PVTCLKSGAICHPVFCPRRYKQIGTCGLPGTKCCKKP

SEQ ID NO: 537 - protein, 38 aa, Bos taurus
APLSCGRNGGVCIPIRCPVPMRQIGTCFGRPVKCCRSW

SEQ ID NO: 538 - protein, 38 aa, Aptenodytes patagonicus
SFGLCRLRRGFCARGRCRFPSIPIGRCSRFVQCCRRVW

SEQ ID NO: 539 - protein, 41 aa, Homo sapiens
GIGDPVTCLKSGAICHPVFCPRRYKQIGTCGLPGTKCCKKP

SEQ ID NO: 540 - protein, 40 aa, Homo sapiens
GIGDPVTCLKSGAICHPVFCPRRYKQIGTCGLPGTKCCKK

SEQ ID NO: 541 - protein, 41 aa, Homo sapiens; identical to SEQ ID NO: 539

SEQ ID NO: 542 - protein, 37 aa, Mus musculus
NSKRACYREGGECLQRCIGLFHKIGTCNFRFKCCKFQ

SEQ ID NO: 543 - protein, 35 aa, Mus musculus
NEPVSCIRNGGICQYRCIGLRHKIGTCGSPFKCCK

SEQ ID NO: 544 - protein, 36 aa, Homo sapiens; identical to SEQ ID NO: 531

SEQ ID NO: 545 - protein, 45 aa, Homo sapiens
GIINTLQKYYCRVRGGRCAVLSCLPKEEQIGKCSTRGRKCCRRKK

SEQ ID NO: 546 - protein, 39 aa, Rhipicephalus bursa
NECVSKGFGCLPQSDCPQEARLSYGGCSTVCCDLSKLTG

SEQ ID NO: 547 - protein, 42 aa, Anthopleura elegantissima
GTTCYCGKTIGIYWFGTKTCPSNRGYTGSCGYFLGICCYPVD

SEQ ID NO: 548 - protein, 9 aa, Vigna radiata var. radiata
CTKSIPPEC

SEQ ID NO: 549 - protein, 10 aa, Artificial Sequence
  Synthetic Construct
CTASIPPQCY

SEQ ID NO: 550 - protein, 10 aa, Artificial Sequence
  Synthetic Construct
VCCGYKLCHC

SEQ ID NO: 551 - protein, 10 aa, Artificial Sequence
  Synthetic Construct
CHWEGNKLVC

SEQ ID NO: 552 - protein, 10 aa, Artificial Sequence
  Synthetic Construct
CTWEGNKLTC

SEQ ID NO: 553 - protein, 10 aa, Artificial Sequence
  Synthetic Construct
CVWEGNKLHC

SEQ ID NO: 554 - protein, 10 aa, Triticum aestivum
SCTRSIPPQC

SEQ ID NO: 555 - protein, 11 aa, Artificial Sequence
  Synthetic Construct
CHWLRGDMRRC

SEQ ID NO: 556 - protein, 11 aa, Macrotyloma axillare
SCTKSIPPQCY

SEQ ID NO: 557 - protein, 11 aa, Artificial Sequence
  Synthetic Construct
CTWEPDGKLTC

SEQ ID NO: 558 - protein, 11 aa, Artificial Sequence
  Synthetic Construct
CYCSLRGDCYC

SEQ ID NO: 559 - protein, 12 aa, Conus marmoreus
NGVCCGYKLCHC

SEQ ID NO: 560 - protein, 12 aa, Artificial Sequence
  Synthetic Construct
RRCYQKPYRCRG

SEQ ID NO: 561 - protein, 12 aa, Artificial Sequence
  Synthetic Construct
SCHFGPLGWVCK

SEQ ID NO: 562 - protein, 12 aa, Homo sapiens
CHWDLLVRHWVC

SEQ ID NO: 563 - protein, 10 aa, Artificial Sequence
  Synthetic Construct
YCKFETFKSC

SEQ ID NO: 564 - protein, 10 aa, Conus imperialis
GCSDPRAWRC

SEQ ID NO: 565 - protein, 10 aa, Conus marmoreus
VCCGYKLCHC

SEQ ID NO: 566 - protein, 18 aa, Artificial Sequence
  Synthetic Construct
RGWVEICAADDYGRCLTE

SEQ ID NO: 567 - protein, 20 aa, Artificial Sequence
  Synthetic Construct
RGWVEICAADDYGRCLTEAQ

SEQ ID NO: 568 - protein, 21 aa, Streptomyces sp. (strain SP9440)
CLGIGSCNDFAGCGYAVVCFW

SEQ ID NO: 569 - protein, 20 aa, Homo sapiens
CEAPEGYILDDGFICTDIDE

SEQ ID NO: 570 - protein, 46 aa, Crambe hispanica subsp. abyssinica
TTCCPSIVARSNFNVCRLPGTPEALCATYTGCIIIPGATCPGDYAN

SEQ ID NO: 571 - protein, 46 aa, Crambe hispanica subsp. abyssinica
TTCCPSIVARSNFNVCRLPGTSEAICATYTGCIIIPGATCPGDYAN

SEQ ID NO: 572 - protein, 46 aa, Viscum album
KSCCPNTTGRNIYNTCRFAGGSRERCAKLSGCKIISASTCPSDYPK

SEQ ID NO: 573 - protein, 46 aa, Crambe hispanica subsp. abyssinica
TTCCPSIVARSNFNVCRLPGTPEAICATYTGCIIIPGATCPGDYAN

SEQ ID NO: 574 - protein, 46 aa, Viscum album
KSCCPNTTGRDIYNTCRLGGGSRERCASLSGCKIISASTCPSDYPK

SEQ ID NO: 575 - protein, 46 aa, Viscum album
KSCCPSTTGRNIYNTCRLTGSSRETCAKLSGCKIISASTCPSNYPK

SEQ ID NO: 576 - protein, 46 aa, Viscum album
KSCCPNTTGRNIYNACRLTGAPRPTCAKLSGCKIISGSTCPSDYPK

SEQ ID NO: 577 - protein, 46 aa, Viscum album
KSCCPNTTGRNIYNTCRFGGGSRQVCASLSGCKIISASTCPSDYPK

SEQ ID NO: 578 - protein, 46 aa, Crambe hispanica subsp. abyssinica; identical to SEQ ID NO: 570

SEQ ID NO: 579 - protein, 46 aa, Crambe hispanica subsp. abyssinica; identical to SEQ ID NO: 570

SEQ ID NO: 580 - protein, 45 aa, Hordeum vulgare
KSCCRSTLGRNCYNLCRVRGAQKLCANACRCKLTSGLKCPSSFPK

SEQ ID NO: 581 - protein, 45 aa, Triticum aestivum
KSCCKSTLGRNCYNLCRARGAQKLCANVCRCKLTSGLSCPKDFPK

SEQ ID NO: 582 - protein, 45 aa, Triticum aestivum
KSCCRSTLGRNCYNLCRARGAQKLCAGVCRCKISSGLSCPKGFPK

SEQ ID NO: 583 - protein, 46 aa, Viscum album
KSCCPNTTGRNIYNTCRLGGGSRERCASLSGCKIISASTCPSDYPK

SEQ ID NO: 584 - protein, 46 aa, Helleborus purpurascens
KSCCRNTLARNCYNACRFTGGSQPTCGILCDCIHVTTTTCPSSHPS

SEQ ID NO: 585 - protein, 46 aa, Crambe hispanica subsp. abyssinica; identical to SEQ ID NO: 570

SEQ ID NO: 586 - protein, 46 aa, Crambe hispanica subsp. abyssinica; identical to SEQ ID NO: 570

SEQ ID NO: 587 - protein, 43 aa, Crambe hispanica subsp. abyssinica
TTCCPSIVARSNFNVCRLPGTPEALCATYTGCIIIPGATCPGD

SEQ ID NO: 588 - protein, 46 aa, Crambe hispanica subsp. abyssinica; identical to SEQ ID NO: 571

SEQ ID NO: 589 - protein, 34 aa, Homo sapiens
GSYDAYRTDCEELSGARTFRLAPAQWSCRETFQA

SEQ ID NO: 590 - protein, 34 aa, Artificial Sequence; identical to SEQ ID NO: 2
  Synthetic Construct

SEQ ID NO: 591 - protein, 34 aa, Artificial Sequence; identical to SEQ ID NO: 2
  Synthetic Construct

SEQ ID NO: 592 - protein, 34 aa, Homo sapiens
CWLCRALIKRIQAMIPKGGRMLPQLVCRLVLRCS

SEQ ID NO: 593 - protein, 35 aa, Human immunodeficiency virus type 1 group M subtype B (isolate JRCSF)
CTRPNNNTRKSIHIGPGRAFYTTGEIIGDIRQAHC

SEQ ID NO: 594 - protein, 38 aa, Homo sapiens
DPCQKQAAEIQKCLQANSYLESKCQAVIQELKKCAAQY

SEQ ID NO: 595 - protein, 32 aa, Anguilla japonica
CSNLSTCVLGKLSQELHKLQTYPRTDVGAGTP

SEQ ID NO: 596 - protein, 34 aa, Ectatomma tuberculatum
WSTIVKLTICPTLKSMAKKCEGSIATMIKKKCDK

SEQ ID NO: 597 - protein, 34 aa, Homo sapiens; identical to SEQ ID NO: 592

SEQ ID NO: 598 - protein, 43 aa, Luffa cylindrica
GSPRTEYEACRVRCQVAEHGVERQRRCQQVCEKRLREREGRRE

SEQ ID NO: 599 - protein, 32 aa, Anguilla japonica; identical to SEQ ID NO: 595

SEQ ID NO: 600 - protein, 32 aa, Anguilla japonica; identical to SEQ ID NO: 595

SEQ ID NO: 601 - protein, 32 aa, Homo sapiens
CGNLSTCMLGTLTQDFHKFHTFPQTNTGVGTP

SEQ ID NO: 602 - protein, 32 aa, Oncorhynchus keta
CSNLSTCVLGKLSQELHKLQTYPRTNTGSGTP

SEQ ID NO: 603 - protein, 32 aa, Oncorhynchus keta
CSNLSTCVLGKLSQELHKLQTYLATNTGSGTP

SEQ ID NO: 604 - protein, 33 aa, Saccharomyces cerevisiae (strain ATCC 204508 / S288c)
NELDVPEQVDKLIQQATSIERLCQHYIGWCPFW

SEQ ID NO: 605 - protein, 32 aa, Rana luteiventris
GILSSFKGVAKGVAKDLAGKLLETLKCKITGC

SEQ ID NO: 606 - protein, 33 aa, Saccharomyces cerevisiae (strain ATCC 204508 / S288c); identical to SEQ ID NO: 604

SEQ ID NO: 607 - protein, 25 aa, Conus planorbis
FPRPRICNLACRAGIGHKYPFCHCR

SEQ ID NO: 608 - protein, 44 aa, Homo sapiens
GSHSCSSTQFKCNSGRCIPEHWTCDGDNDCGDYSDETHANCTNQ

SEQ ID NO: 609 - protein, 46 aa, Homo sapiens
AVGDRCERNEFQCQDGKCISYKWVCDGSAECQDGSDESQETCLSVT

SEQ ID NO: 610 - protein, 45 aa, Homo sapiens
GSPPQCQPGEFACANSRCIQERWKCDGDNDCLDNSDEAPALCHQH

SEQ ID NO: 611 - protein, 39 aa, Homo sapiens
CRIHEISCGAHSTQCIPVSWRCDGENDCDSGEDEENCGN

SEQ ID NO: 612 - protein, 37 aa, Homo sapiens
PCSAFEFHCLSGECIHSSWRCDGGPDCKDKSDEENCA

SEQ ID NO: 613 - protein, 35 aa, Homo sapiens
CEKDQFQCRNERCIPSVWRCDEDDDCLDHSDEDDC

SEQ ID NO: 614 - protein, 38 aa, Homo sapiens
TCGPASFQCNSSTCIPQLWACDNDPDCEDGSDEWPQRC

SEQ ID NO: 615 - protein, 39 aa, Homo sapiens
ATCRPDEFQCSDGNCIHGSRQCDREYDCKDMSDEVGCVN

SEQ ID NO: 616 - protein, 40 aa, Homo sapiens
VATCRPDEFQCSDGNCIHGSRQCDREYDCKDLSDEVGCVN

SEQ ID NO: 617 - protein, 42 aa, Homo sapiens
PGGCHTDEFQCRLDGLCIPLRWRCDGDTDCMDSSDEKSCEGV

SEQ ID NO: 618 - protein, 43 aa, Homo sapiens
GSQDVKCSLGYFPCGNITKCLPQLLHCNGVDDCGNQADEDNCG

SEQ ID NO: 619 - protein, 41 aa, Homo sapiens
LSVTCKSGDFSCGGRVNRCIPQFWRCDGQVDCDNGSDEQGC

SEQ ID NO: 620 - protein, 42 aa, Coturnix coturnix japonica
SCPPGQFRCSEPPGAHGECYPQDWLCDGHPDCDDGRDEWGCG

SEQ ID NO: 621 - protein, 48 aa, Rattus norvegicus
GAMVLNCTSAQFKCADGSSCINSRYRCDGVYDCRDNSDEAGCPTRPPG

SEQ ID NO: 622 - protein, 25 aa, Conus ermineus
GCCGPYNAACHCGCKVGRYCDRSGG

SEQ ID NO: 623 - protein, 36 aa, Hypocrea jecorina; identical to SEQ ID NO: 367

SEQ ID NO: 624 - protein, 31 aa, Hydra vulgaris; identical to SEQ ID NO: 415

SEQ ID NO: 625 - protein, 29 aa, Mesobuthus martensii
ATCEDCPEHCATQNARAKCDNDKCVCEPK

SEQ ID NO: 626 - protein, 31 aa, Homo sapiens
VVHCDMEVICPDGYTCCRLPSGAWGCCPFTQ

SEQ ID NO: 627 - protein, 35 aa, Oryza sativa subsp. japonica
DHVCDDNFSCPAGSTCSSAFGFRNLSLVWGCSPVE

SEQ ID NO: 628 - protein, 30 aa, Cyprinus carpio
VIHCDAATICPDGTTCSLSPYGVWYCSPFS

SEQ ID NO: 629 - protein, 34 aa, Scorpio maurus palmatus
VSCTGSKDCYAPCRKQTGCPYGKCMNRKCKCNRC

SEQ ID NO: 630 - protein, 33 aa, Leiurus quinquestriatus hebraeus
AFCNLRMCQLSCRKSLGCLLGKCIGDKCKCYGC

SEQ ID NO: 631 - protein, 28 aa, Mesobuthus martensii
VGCEECPMHCKGKNAKPTCDDGVCNCNV

SEQ ID NO: 632 - protein, 29 aa, Androctonus mauretanicus mauretanicus
VSCEDCPEHCSTQKAQAKCDNDKCVCEPI

SEQ ID NO: 633 - protein, 28 aa, Mesobuthus martensii
VGCEECPMHCKGKNANPTCDDGVCNCNV

SEQ ID NO: 634 - protein, 28 aa, Cyprinus carpio
HCDAATICPDGTTCSLSPYGVWYCSPFS

SEQ ID NO: 635 - protein, 26 aa, Corynespora cassiicola
TCVSCVNFGNGFCGDNCGNSWACSGC

SEQ ID NO: 636 - protein, 34 aa, Mytilus edulis
SCASRCKGHCRARRCGYYVSVLYRGRCYCKCLRC

SEQ ID NO: 637 - protein, 33 aa, Schistocerca gregaria
QECTPGQTKKQDCNTCNCTPTGVWACTRKGCPP

SEQ ID NO: 638 - protein, 32 aa, Locusta migratoria
KCTPGQVKQQDCNTCTCTPTGVWGCTLMGCQP

SEQ ID NO: 639 - protein, 33 aa, Locusta migratoria
SCEPGKTFKDKCNTCRCGADGKSAACTLKACPN

SEQ ID NO: 640 - protein, 33 aa, Locusta migratoria migratorioides
ECTPGQTKKQDCNTCTCTPTGIWGCTRKACRTT

SEQ ID NO: 641 - protein, 35 aa, Schistocerca gregaria
QECEPGQTKKQDCNTCRCGSDGVWACTRMGCPPHA

SEQ ID NO: 642 - protein, 35 aa, Schistocerca gregaria
EVTCEPGTTFKDKCNTCRCGSDGKSAACTRMACPQ

SEQ ID NO: 643 - protein, 35 aa, Schistocerca gregaria
EVTCEPGTTFKDKCNTCRCGSDGKSAACTLKACPQ

SEQ ID NO: 644 - protein, 34 aa, Locusta migratoria
ISCEPGKTFKDKCNTCRCGADGKSAACTLKACPN

SEQ ID NO: 645 - protein, 36 aa, Locusta migratoria
EISCEPGKTFKDKCNTCRCGADGKSAACTLKACPNQ

SEQ ID NO: 646 - protein, 35 aa, Locusta migratoria migratorioides
AGECTPGQTKKQDCNTCTCTPTGIWGCTRKACRTT

SEQ ID NO: 647 - protein, 35 aa, Schistocerca gregaria
EQECTPGQTKKQDCNTCNCTPTGVWACTRKGCPPH

SEQ ID NO: 648 - protein, 38 aa, Homo sapiens
SLSCRKEQGKFYDHLLRDCISCASICGQHPKQCAYFCE

SEQ ID NO: 649 - protein, 36 aa, Homo sapiens
CRKEQGKFYDHLLRDCISCASICGQHPKQCAYFCEN

SEQ ID NO: 650 - protein, 39 aa, Homo sapiens
CRKEQGKFYDHLLRDCISCASICGQHPKQCAYFCENKLR

SEQ ID NO: 651 - protein, 35 aa, Homo sapiens
CSQNEYFDSLLHACIPCQLRCSSNTPPLTCQRYCN

SEQ ID NO: 652 - protein, 36 aa, Homo sapiens
CSQNEYFDSLLHACIPCQLRCSSNTPPLTCQRYCNA

SEQ ID NO: 653 - protein, 37 aa, Homo sapiens
GQCSQNEYFDSLLHACIPCQLRCSSNTPPLTCQRYCN

SEQ ID NO: 654 - protein, 39 aa, Homo sapiens
CSQNEYFDSLLHACIPCQLRCSSNTPPLTCQRYCNASVT

SEQ ID NO: 655 - protein, 46 aa, Homo sapiens
GSPWSLSCRKEQGKFYDHLLRDCISCASICGQHPKQCAYFCENKLR

SEQ ID NO: 656 - protein, 18 aa, Torpedo californica
YRGWKHWVYYTCCPDTPY

SEQ ID NO: 657 - protein, 24 aa, Homo sapiens
ANSFLLRHSSLRCIICDFAKIFQN

SEQ ID NO: 658 - protein, 24 aa, Homo sapiens
ANSFLLRHSSLRCIICDFAKIFQN

SEQ ID NO: 659 - protein, 19 aa, Conus geographus
RDCCTKKCKDAQCKQRCCA

SEQ ID NO: 660 - protein, 19 aa, Conus geographus
RDCCTKKCKDRQCKQRCCA

SEQ ID NO: 661 - protein, 19 aa, Conus geographus
RDCCTRKCKDRRCKMKCCA

SEQ ID NO: 662 - protein, 18 aa, Conus purpurascens
RLCCGFKSCRSRQCKHRC

SEQ ID NO: 663 - protein, 17 aa, Pseudomonas aeruginosa (strain ATCC 15692 / PAO1 / 1C / PRS 101 / LMG 12228)
ACKSTQDPMFTPKGCDN

SEQ ID NO: 664 - protein, 19 aa, Artificial Sequence
  Synthetic Construct
GGNECDIARMWEWECFERL

SEQ ID NO: 665 - protein, 21 aa, Apis mellifera
ALCNCNRIIIPHMCWKKCGKK

SEQ ID NO: 666 - protein, 21 aa, Conus purpurascens
HCCLYGKCRRYGCSSASCCQR

SEQ ID NO: 667 - protein, 20 aa, Conus purpurascens
GCCLYGSCRFGCYNALCCRK

SEQ ID NO: 668 - protein, 18 aa, Torpedo californica
YRGWKHWVYYTCCPDTPY

SEQ ID NO: 669 - protein, 40 aa, Hirudo medicinalis
KVCACPKILKPVCGSDGRTYANSCIARCNGVSIKSEGSCP

SEQ ID NO: 670 - protein, 44 aa, Hirudo medicinalis
KKVCACPKILKPVCGSDGRTYANSCIARCNGVSIKSEGSCPTGI

SEQ ID NO: 671 - protein, 44 aa, Hirudo medicinalis; identical to SEQ ID NO: 670

SEQ ID NO: 672 - protein, 44 aa, Hirudo medicinalis; identical to SEQ ID NO: 670

SEQ ID NO: 673 - protein, 39 aa, Carcinoscorpius rotundicauda
SCPHTYKPVCGANGEVYDNECFLNKAGIEPAESWETCRG

SEQ ID NO: 674 - protein, 39 aa, Carcinoscorpius rotundicauda
CPHTYKPVCGANGEVYDNECFLNKAGIEPAESWETCRGH

SEQ ID NO: 675 - protein, 48 aa, Anemonia sulcata
KPDAPCICTMQYDPVCGSDGITYGNACMLLCASARSDTPIELVHKGRC

SEQ ID NO: 676 - protein, 48 aa, Anemonia sulcata
KPDCPLICTMQYDPVCGSDGITYGNACMLLGASCRSDTPIELVHKGRC

SEQ ID NO: 677 - protein, 44 aa, Hirudo medicinalis; identical to SEQ ID NO: 670

SEQ ID NO: 678 - protein, 27 aa, Mus musculus
EKPFSCSLCPQRSRDFSAMTKHLRTHG

SEQ ID NO: 679 - protein, 30 aa, Amaranthus caudatus
VGECVRGRCPSGMCCSQFGYCGKGPKYCGR

SEQ ID NO: 680 - protein, 30 aa, Amaranthus caudatus
VGECVRGRCPSGMCCSQWGYCGKGPKYCGR

SEQ ID NO: 681 - protein, 29 aa, Amaranthus caudatus
VGECVRGRCPSGMCCSQGYCGKGPKYCGR

SEQ ID NO: 682 - protein, 28 aa, Amaranthus caudatus
VGECVRGRCPSGMCCSQGCGKGPKYCGR

SEQ ID NO: 683 - protein, 32 aa, Hevea brasiliensis
EQCGRQAGGKLCPNNLCCSQWGWCGSTDEYCS

SEQ ID NO: 684 - protein, 35 aa, Stellaria media
SGPNGQCGPGWGGCRGGLCCSQYGYCGSGPKYCAH

SEQ ID NO: 685 - protein, 31 aa, Homo sapiens
PTPCVPAECFDLLVRHCVACGLLRTPRPKPA

SEQ ID NO: 686 - protein, 43 aa, Hevea brasiliensis
EQCGRQAGGKLCPNNLCCSQWGWCGSTDEYCSPDHNCQSNCKD

SEQ ID NO: 687 - protein, 43 aa, Hevea brasiliensis
EQCGRQAGGKLCPDNLCCSQWGWCGSTDEYCSPDHNCQSNCKD

SEQ ID NO: 688 - protein, 49 aa, Anthopleura xanthogrammica
GVSCLCDSDGPSVRGNTLSGTLWLYPSGCPSGWHNCKAHGPTIGWCCKQ

SEQ ID NO: 689 - protein, 49 aa, Anthopleura xanthogrammica
GVPCLCDSDGPRPRGNTLSGILWFYPSGCPSGWHNCKAHGPNIGWCCKK

SEQ ID NO: 690 - protein, 46 aa, Condylactis gigantea
GVCRCDSDGPSVHGNTLSGTVWVGSCASGWHKCNDEYNIAYECCKQ

SEQ ID NO: 691 - protein, 48 aa, Stoichactis helianthus
AACKCDDEGPDIRTAPLTGTVDLGSCNAGWEKCASYYTIIADCCRKKK

SEQ ID NO: 692 - protein, 48 aa, Stoichactis helianthus; identical to SEQ ID NO: 691

SEQ ID NO: 693 - protein, 48 aa, Stoichactis helianthus; identical to SEQ ID NO: 691

SEQ ID NO: 694 - protein, 46 aa, Anemonia sulcata
GAACLCKSDGPNTRGNSMSGTIWVFGCPSGWNNCEGRAIIGYCCKQ

SEQ ID NO: 695 - protein, 43 aa, Anemonia sulcata
AAPCFCSGKPGRGDLWILRGTCPGGYGYTSNCYKWPNICCYPH

SEQ ID NO: 696 - protein, 10 aa, Homo sapiens
KCNCALANFL

SEQ ID NO: 697 - protein, 35 aa, Thermus thermophilus (strain HB8 / ATCC 27634 / DSM 579)
KVRSSVKKMCDNCKVVRRHGRVLVICSNVKHKQRQ

SEQ ID NO: 698 - protein, 35 aa, Artificial Sequence
  Synthetic Construct
KVRASVKRICDKCKVIRRHGRVYVICENPKHKQRQ

SEQ ID NO: 699 - protein, 36 aa, Thermus thermophilus (strain HB8 / ATCC 27634 / DSM 579)
KVRASVKRICDKCKVIRRHGRVYVICENPKHKQRQG

SEQ ID NO: 700 - protein, 37 aa, Thermus thermophilus (strain HB8 / ATCC 27634 / DSM 579)
MKVRASVKRICDKCKVIRRHGRVYVICENPKHKQRQG

SEQ ID NO: 701 - protein, 37 aa, Thermus thermophilus (strain HB8 / ATCC 27634 / DSM 579); identical to SEQ ID NO: 700

SEQ ID NO: 702 - protein, 38 aa, Escherichia coli (strain K12) [sequence omitted]

SEQ ID NO: 703 - protein, 38 aa, Escherichia coli (strain K12) [sequence omitted]

SEQ ID NO: 704 - protein, 38 aa, Escherichia coli (strain K12) [sequence omitted]

SEQ ID NO: 705 - protein, 37 aa, Bunodosoma granulifera
VCRDWFKETACRHAKSLGNCRTSQKYRANCAKTCELC

SEQ ID NO: 706 - protein, 37 aa, Rattus norvegicus
YGCLDRIFVCTSWARKGFCDVRQRLMKRLCPRSCDFC

SEQ ID NO: 707 - protein, 35 aa, Stoichactis helianthus
RSCIDTIPKSRCTAFQCKHSMKYRLSFCRKTCGTC

SEQ ID NO: 708 - protein, 34 aa, Stoichactis helianthus
RSCIDTIPKSRCTAFQCKHSMYRLSFCRKTCGTC

SEQ ID NO: 709 - protein, 34 aa, Stoichactis helianthus
RSCIDTIPKSRCTAFQCKHSKYRLSFCRKTCGTC

SEQ ID NO: 710 - protein, 35 aa, Homo sapiens
EEACELPECQEDAGNKVCSLQCNNHACGWDGGDCS

SEQ ID NO: 711 - protein, 37 aa, Ectatomma tuberculatum
GVIPKKIWETVCPTVEPWAKKCSGDIATYIKRECGKL

SEQ ID NO: 712 - protein, 31 aa, Hydra vulgaris
GSQITGTCPSVCSGDCYPECPPGCCGQVNLN

SEQ ID NO: 713 - protein, 21 aa, Atractaspis engaddensis
CSCKDMTDKECLYFCHQDVIW

SEQ ID NO: 714 - protein, 22 aa, Homo sapiens
YNCEPANPSEKNSPSTQYCYSI

SEQ ID NO: 715 - protein, 24 aa, Homo sapiens
IYNCEPANPSEKNSPSTQYCYSIQ

SEQ ID NO: 716 - protein, 20 aa, Homo sapiens
CEPANPSEKNSPSTQYCYSI

SEQ ID NO: 717 - protein, 17 aa, Homo sapiens
CEPANPSEKNSPSTQYC

SEQ ID NO: 718 - protein, 15 aa, Conus aulicus
GCCSYPPCFATNPDC

SEQ ID NO: 719 - protein, 25 aa, Saccharomyces cerevisiae (strain ATCC 204508 / S288c)
EFCLNCVSHTASYLRLWALSLAHAQ

SEQ ID NO: 720 - protein, 42 aa, Pelodiscus sinensis
DDTPSSRCGSGGWGPCLPIVDLLCIVHVTVGCSGGFGCCRIG

SEQ ID NO: 721 - protein, 42 aa, Crotalus durissus terrificus
YKQCHKKGGHCFPKEKICLPPSSDFGKMDCRWRWKCCKKGSG

SEQ ID NO: 722 - protein, 41 aa, Ornithorhynchus anatinus
IFFEMQACWSHSGVCRDKSERNCKPMAWTYCENRNQKCCEY

SEQ ID NO: 723 - protein, 42 aa, Ornithorhynchus anatinus
IMFFEMQACWSHSGVCRDKSERNCKPMAWTYCENRNQKCCEY

SEQ ID NO: 724 - protein, 42 aa, Ornithorhynchus anatinus
FVQHRPRDCESINGVCRHKDTVNCREIFLADCYNDGQKCCRK

SEQ ID NO: 725 - protein, 42 aa, Ornithorhynchus anatinus; identical to SEQ ID NO: 723

SEQ ID NO: 726 - protein, 21 aa, Artificial Sequence
  Synthetic Construct
VQCPHFCYELDYELCPDVCYV

SEQ ID NO: 727 - protein, 21 aa, Artificial Sequence
  Synthetic Construct
ALCPAVCYVGGKALCPDVCYV

SEQ ID NO: 728 - protein, 21 aa, Artificial Sequence
  Synthetic Construct
VQCPHFCYELDYELCPDVCYV

SEQ ID NO: 729 - protein, 24 aa, Hydra sp.
PCPPVCVAQCVPTCPQYCCPAKRK

SEQ ID NO: 730 - protein, 24 aa, Hydra sp.
PCPPVCVAQCVPTCPQYCCPAKRK

SEQ ID NO: 731 - protein, 23 aa, Artificial Sequence
  Synthetic Construct
CAEPMTLPENYFSERPYHPPPPC

SEQ ID NO: 732 - protein, 8 aa, Artificial Sequence
  Synthetic Construct
CKFFTTSC

SEQ ID NO: 733 - protein, 9 aa, Artificial Sequence
  Synthetic Construct
YCKFETFSC

SEQ ID NO: 734 - protein, 8 aa, Artificial Sequence
  Synthetic Construct
CKFATTSC

SEQ ID NO: 735 - protein, 9 aa, Artificial Sequence
  Synthetic Construct
YCKFETFSC

SEQ ID NO: 736 - protein, 10 aa, Artificial Sequence
  Synthetic Construct
YCKEFTFKSC

SEQ ID NO: 737 - protein, 8 aa, Conus textile
CCDGCCAA

SEQ ID NO: 738 - protein, 37 aa, Euplotes raikovi
DICDIAIAQCSLTLCQDCENTPICELAVKGSCPPPWS

SEQ ID NO: 739 - protein, 39 aa, Euplotes raikovi
DECANAAAQCSITLCNLYCGPLIEICELTVMQNCEPPFS

SEQ ID NO: 740 - protein, 38 aa, Euplotes raikovi
DLCEQSALQCNEQGCHNFCSPEDKPGCLGMVWNPELCP

SEQ ID NO: 741 - protein, 40 aa, Euplotes raikovi
DACEQAAIQCVESACESLCTEGEDRTGCYMYIYSNCPPYV

SEQ ID NO: 742 - protein, 40 aa, Euplotes raikovi
DPMTCEQAMASCEHTMCGYCQGPLYMTCIGITTDPECGLP

SEQ ID NO: 743 - protein, 34 aa, Sus scrofa
PDPLPRRVCLNPDCDELADHIGFQEAYRRFYGIA

SEQ ID NO: 744 - protein, 31 aa, Bos taurus
LPKRVCLNPDCDELADHIGFQEAYRRFYGPV

SEQ ID NO: 745 - protein, 38 aa, Argyrosomus regius
ELTLAQTSLRVCTNMACDMADAQGIVAAYQAFYGPIPF

SEQ ID NO: 746 - protein, 39 aa, Argyrosomus regius
KELTLAQTSLRVCTNMACDMADAQGIVAAYQAFYGPIPF

SEQ ID NO: 747 - protein, 44 aa, Protobothrops jerdonii
AMDCTTGPCCRQCKLKPAGTTCWRTSVSSHYCTGRSCECPSYPG

SEQ ID NO: 748 - protein, 44 aa, Protobothrops jerdonii
AMDCTTGPCCRQCKLKPAGTTCWKTSVSSHYCTGRSCECPSYPG

SEQ ID NO: 749 - protein, 46 aa, Protobothrops jerdonii
AMDCTTGPCCRQCKLKPAGTTCWKTSVSSHYCTGRSCECPSYPGNG

SEQ ID NO: 750 - protein, 46 aa, Protobothrops jerdonii
AMDCTTGPCCRQCKLKPAGTTCWRTSVSSHYCTGRSCECPSYPGNG

SEQ ID NO: 751 - protein, 47 aa, Homo sapiens
LPASFDAREQWPQCPTIKEIRDQGSCGSCWAFGAVEAISDRICIHTN

SEQ ID NO: 752 - protein, 47 aa, Homo sapiens; identical to SEQ ID NO: 751

SEQ ID NO: 753 - protein, 48 aa, Bos taurus
LPESFDAREQWPNCPTIKEIRDQGSCGSCWAFGAVEAISDRICIHSNG

SEQ ID NO: 754 - protein, 35 aa, Homo sapiens
YNSGKLFVQGNLRCMKCSFARVFNTRTTFWKQYVD

SEQ ID NO: 755 - protein, 34 aa, Bos taurus
YNSGKLFVRGNLRCKKCSFARVFNTKTTFWKQYV

SEQ ID NO: 756 - protein, 32 aa, Bos taurus
ANSFLVKQGNLRCLACSLARVFDAQTDFWSKY

SEQ ID NO: 757 - protein, 37 aa, Homo sapiens [sequence omitted]

SEQ ID NO: 758 - protein, 37 aa, Glandirana rugosa
GILDTLKQFAKGVGKDLVKGAAQGVLSTVSCKLAKTC

SEQ ID NO: 759 - protein, 40 aa, Homo sapiens
QESCKGRCTEGFNVDKKCQCDELCSYYQSCCTDYTAECKP

SEQ ID NO: 760 - protein, 40 aa, Homo sapiens; identical to SEQ ID NO: 759

SEQ ID NO: 761 - protein, 46 aa, Piromyces equi
ASCWAQSQGYNCCNNPSSTKVEYTDASGQWGVQNGQWCGIDYSYGQ

SEQ ID NO: 762 - protein, 46 aa, Piromyces equi; identical to SEQ ID NO: 761

SEQ ID NO: 763 - protein, 42 aa, Conus radiatus
GSFCKADEKCEYHADCCNCCLSGICASTNWILPGCSTSSFKI

SEQ ID NO: 764 - protein, 43 aa, Conus radiatus
GSFCKADEKCEYHADCCNCCLSGICASTNWILPGCSTSSFFKI

SEQ ID NO: 765 - protein, 40 aa, Ambrosia trifida
DDGLCYEGTNCGKVGKYCCSPIGKYCVCYDSKAICNKNCT

SEQ ID NO: 766 - protein, 40 aa, Ambrosia trifida; identical to SEQ ID NO: 765

SEQ ID NO: 767 - protein, 45 aa, Camelus dromedarius
ASKKSVRWCTTSPAESKKCAQWQRRMKKVRGPSVTCVKKTSRFEC

SEQ ID NO: 768 - protein, 45 aa, Camelus dromedarius; identical to SEQ ID NO: 767

SEQ ID NO: 769 - protein, 40 aa, Homo sapiens; identical to SEQ ID NO: 216

SEQ ID NO: 770 - protein, 40 aa, Homo sapiens
MTPFWRGVSLRPIGASCRDDSECITRLCRKRRCSLSVAQE

SEQ ID NO: 771 - protein, 43 aa, Bos taurus
ARIIRYFYNAKAGLCQTFVYGGCRAKRNNFKSAEDCMRTCGGA

SEQ ID NO: 772 - protein, 43 aa, Lactobacillus sakei
KYYGNGVHCGKHSCTVDWGTAIGNIGNNAAANWATGGNAGWNK

SEQ ID NO: 773 - protein, 45 aa, Homo sapiens
ALARCPGCGQGVQAGCPGGCVEEEDGGSPAEGCAEAEGCLRREGQ

SEQ ID NO: 774 - protein, 48 aa, Escherichia coli
STQSNKKDLCEHYRQIAKESCKKGFLGVRDGTAGACFGAQIMVAAKGC

SEQ ID NO: 775 - protein, 43 aa, Sulfolobus shibatae B12
CGKCWKTFTDEQLKVLPGVRCPYCGYKIIFMVRKPTIKIVKAI

SEQ ID NO: 776 - protein, 47 aa, Litopenaeus setiferus
HSSGYTRPLRKPSRPIFIRPIGCDVCYGIPSSTARLCCFRYGDCCHL

SEQ ID NO: 777 - protein, 33 aa, Homo sapiens
QPLPDCCRQKTCSCRLYELLHGAGNHAAGILTL

SEQ ID NO: 778 - protein, 31 aa, Drosophila melanogaster
WEWPWNRKTKFISNRDKWCRLNLGPAWGGRC

SEQ ID NO: 779 - protein, 36 aa, Haliotis rufescens
TRHSFRRPFHECALCYSITDPGERQRCIDMYCSYTN

SEQ ID NO: 780 - protein, 39 aa, Mus musculus
KEHCGSILHGTWLPKKCSLCRCWHGQLHCLPQTFLPGCD

SEQ ID NO: 781 - protein, 44 aa, Lactobacillus sakei
KYYGNGVHCGKHSCTVDWGTAIGCIGNNAAANWATGGNAGWNKC

SEQ ID NO: 782 - protein, 49 aa, Echis carinatus sochureki
ECESGPCCRNCKFLKEGTICKRARGDDMDDYCNGKTCDCPRNPHKGPAT

SEQ ID NO: 783 - protein, 49 aa, Homo sapiens
GRRRRSVQWCAVSQPEATKCFQWQRNMRKVRGPPVSCIKRDSPIQCIQA

SEQ ID NO: 784 - protein, 49 aa, Homo sapiens
GSSGSSGSCQYQDLLSNCDSLKNTAGCEHELLKEKCKATCLCESGPSSG

SEQ ID NO: 785 - protein, 43 aa, Lactobacillus plantarum
KPAWCWYTLAMCGAGYDSGTCDYMYSHCFGIKHHSSGSSSYHC

SEQ ID NO: 786 - protein, 43 aa, Lactobacillus sakei; identical to SEQ ID NO: 772

SEQ ID NO: 787 - protein, 46 aa, Trypanosoma brucei brucei
GKSPEAECNKITEEPKCSEEKICSWHKEVKAGEKNCQFNSTKASKS

SEQ ID NO: 788 - protein, 48 aa, Plasmodium falciparum (isolate Wellcome)
NISQHQCVKKQCPQNSGCFRHLDEREECKCLLNYKQEGDKCVENPNPT

SEQ ID NO: 789 - protein, 44 aa, Homo sapiens
RCQYGWQGLYCDKCIPHPGCVHGICNEPWQCLCETNWGGQLCDK

SEQ ID NO: 790 - protein, 49 aa, Human immunodeficiency virus type 2 subtype A (isolate Ghana-1)
AQQRKVIRCWNCGKEGHSARQCRAPRRQGCWKCGKTGHVMAKCPERQAG

SEQ ID NO: 791 - protein, 32 aa, Homo sapiens
PLPDCCRQKTCSCRLYELLHGAGNHAAGILTL

SEQ ID NO: 792 - protein, 41 aa, Lactobacillus curvatus
ARSYGNGVYCNNKKCWVNRGEATQSIIGGMISGWASGLAGM

SEQ ID NO: 793 - protein, 48 aa, Homo sapiens
GSAMGCRHFQSCSQCLSAPPFVQCGWCHDKCVRSEECLSGTWTQQICL

SEQ ID NO: 794 - protein, 43 aa, Sus scrofa
IVGGYTCAANSIPYQVSLNSGSHFCGGSLINSQWVVSAAHCYK

SEQ ID NO: 795 - protein, 47 aa, Homo sapiens
YNSGKLEEFVQGNLERECMEEKCSFEEAREVFENTERTTEFWKQYVD

SEQ ID NO: 796 - protein, 45 aa, Anopheles gambiae
ATCDLASFSSQWVTPNDSLCAAHCLVKGYRGGYCKNKICHCRDKF

SEQ ID NO: 797 - protein, 47 aa, Coturnix coturnix japonica
GSSRCPPGQFRCSEPPGAHGECYPQDWLCDGHPDCDDGRDEWGCGTS

SEQ ID NO: 798 - protein, 41 aa, Vipera lebetina obtusa
CTTGPCCRQCKLKPAGTTCWKTSLTSHYCTGKSCDCPLYPG

SEQ ID NO: 799 - protein, 44 aa, Homo sapiens
EPVDPCFRANCEYQCQPLNQTSYLCVCAEGFAPIPHEPHRCQMF

SEQ ID NO: 800 - protein, 45 aa, Bos taurus
DYAKCVFPFIYGGKKYETCTKIGSMWMSWCSLSPNYDKDRAWKYC

SEQ ID NO: 801 - protein, 36 aa, Caretta caretta
EKKCPGRCTLKCGKHERPTLPYNCGKYICCVPVKVK

SEQ ID NO: 802 - protein, 37 aa, Rattus norvegicus
KCNTATCATQRLANFLVRSSNNLGPVLPPTNVGSNTY

SEQ ID NO: 803 - protein, 49 aa, Mus musculus
GSVTHRFSTKSWLSQVCNVCQKSMIFGVKCKHCRLKCHNKCTKEAPACR

SEQ ID NO: 804 - protein, 38 aa, Macrovipera lebetina
GDNKPPKKGPPNGCFGHKIDRIGSHSGLGCNKVDDNKG

SEQ ID NO: 805 - protein, 47 aa, Homo sapiens
MDVVDSLLVNGSNITPPCELGLENETLFCLDQPRPSKEWQPAQVILL

SEQ ID NO: 806 - protein, 48 aa, Trypanosoma brucei brucei
GTKASKSGVPVTQTQTAGADTTAEKCKGKGEKDCKSPDCKWEGGTCKD

SEQ ID NO: 807 - protein, 42 aa, Emericella nidulans (strain FGSC A4 / M139)
TTCTNCFTQTTPLWRRNPEGQPLCNACGLFLKLHGVVRPLSL

SEQ ID NO: 808 - protein, 43 aa, Homo sapiens
CKYKFENWGACDGGTGTKVRQGTLKKARYNAQCQETIRVTKPC

SEQ ID NO: 809 - protein, 48 aa, Carnobacterium maltaromaticum
VNYGNGVSCSKTKCSVNWGQAFQERYTAGINSFVSGVASGAGSIGRRP

SEQ ID NO: 810 - protein, 49 aa, Homo sapiens; identical to SEQ ID NO: 783

SEQ ID NO: 811 - protein, 49 aa, Plasmodium falciparum
GSASCGVWDEWSPCSVTCGKGTRSRKREILHEGCTSEIQEQCEEERCPP

SEQ ID NO: 812 - DNA, 57 bp, Artificial Sequence
  Synthetic construct
ggtggcaacgaatgcgacatcgctcgtatgtgggagtgggaatgcttcgagcgtctg

SEQ ID NO: 813 - DNA, 57 bp, Artificial Sequence
  Modified nucleic acid sequence for disulfide- rich peptide
  misc_feature (19)..(20): n is a, c, g, or t
  misc_feature (22)..(23): n is a, c, g, or t
  misc_feature (25)..(26): n is a, c, g, or t
  misc_feature (28)..(29): n is a, c, g, or t
  misc_feature (31)..(32): n is a, c, g, or t
  misc_feature (37)..(38): n is a, c, g, or t
  misc_feature (46)..(47): n is a, c, g, or t
ggtggcaacgaatgcgacnnknnknnknnknnkgagnnkgaatgcnnkgagcgtctg

SEQ ID NO: 814 - protein, 19 aa, Artificial Sequence
  Modified disulfide-rich peptide
  misc_feature (7)..(11): Xaa can be any naturally occurring amino acid
  misc_feature (13)..(13): Xaa can be any naturally occurring amino acid
  misc_feature (16)..(16): Xaa can be any naturally occurring amino acid
GGNECDXXXXXEXECXERL

SEQ ID NO: 815 - DNA, 48 bp, Artificial Sequence
  Synthetic construct
acctactcctgtcacttcggtccgctgacttgggtttgtaaaccgcag

SEQ ID NO: 816 - DNA, 48 bp, Artificial Sequence
  Modified nucleic acid sequence encoding disulfide-rich peptide
  misc_feature (13)..(14): n is a, c, g, or t
  misc_feature (16)..(17): n is a, c, g, or t
  misc_feature (19)..(20): n is a, c, g, or t
  misc_feature (22)..(23): n is a, c, g, or t
  misc_feature (25)..(26): n is a, c, g, or t
  misc_feature (28)..(29): n is a, c, g, or t
  misc_feature (31)..(32): n is a, c, g, or t
  misc_feature (34)..(35): n is a, c, g, or t
  misc_feature (40)..(41): n is a, c, g, or t
acctactcctgtnnknnknnknnknnknnknnknnktgtnnkccgcag

SEQ ID NO: 817 - protein, 16 aa, Artificial Sequence
  Modified disulfide-rich peptide
  misc_feature (5)..(12): Xaa can be any naturally occurring amino acid
  misc_feature (14)..(14): Xaa can be any naturally occurring amino acid
TYSCXXXXXXXXCXPQ

SEQ ID NO: 818 - DNA, 102 bp, Artificial Sequence
  Synthetic construct
ttcaacatgcagtgccaacgtcgattctatgaagctctgcatgacccgaacctgaacgaagaacaacgtaacgctaaaatcaagagcatccgtgatgactgc

SEQ ID NO: 819 - DNA, 102 bp, Artificial Sequence
  Modified nucleic acid sequence encoding disulfide-rich peptide
  misc_feature (16)..(17): n is a, c, g, or t
  misc_feature (25)..(26): n is a, c, g, or t
  misc_feature (28)..(29): n is a, c, g, or t
  misc_feature (37)..(38): n is a, c, g, or t
  misc_feature (40)..(41): n is a, c, g, or t
  misc_feature (58)..(59): n is a, c, g, or t
  misc_feature (70)..(71): n is a, c, g, or t
  misc_feature (82)..(83): n is a, c, g, or t
  misc_feature (91)..(92): n is a, c, g, or t
ttcaacatgcagtgcnnkcgtcgannknnkgaagctnnknnkgacccgaacctgaacnnkgaacaacgtnnkgctaaaatcnnkagcatcnnkgatgactgc